(19) United States
(12) Patent Application Publication
(10) Pub. No.: US 2013/0096305 A1
(43) Pub. Date: Apr. 18, 2013

(54) DEUTERATED HIV ATTACHMENT INHIBITORS

(71) Applicants: Tao Wang, Farmington, CT (US); Nicholas A. Meanwell, East Hampton, CT (US); Zhongxing Zhang, Madison, CT (US)

(72) Inventors: Tao Wang, Farmington, CT (US); Nicholas A. Meanwell, East Hampton, CT (US); Zhongxing Zhang, Madison, CT (US)

(73) Assignee: Bristol-Myers Squibb Company

(21) Appl. No.: 13/704,288

(22) PCT Filed: Jun. 15, 2011
(86) PCT No.: PCT/US11/40516

§ 371 Date: Dec. 14, 2012

Related U.S. Application Data
(60) Provisional application No. 61/355,788, filed on Jun. 17, 2010.

(51) Int. Cl. C07D 471/04

(52) U.S. Cl. CPC C07D 471/04
USPC 544/362

(57) ABSTRACT

Deuterated piperazine and piperidine HIV attachment inhibitor compounds are set forth. The present invention provides compounds of Formula I, the pharmaceutically acceptable salts and/or solvates (e.g., hydrates) thereof, their pharmaceutical formulations, and their use in patients suffering from or susceptible to a virus such as HIV. The compounds of Formula I, their pharmaceutically acceptable salts and/or solvates are effective antiviral agents, particularly as inhibitors of HIV. They are useful for the treatment of HIV and AIDS.

### FIELD OF THE INVENTION

This invention provides compounds having drug and bio-affecting properties, their pharmaceutical compositions and methods of use. In particular, the invention herein is directed to deuterated HIV attachment inhibitors that possess unique antiviral activity. More particularly, the present invention relates to deuterated piperazine and piperidine compounds useful for the treatment of HIV and AIDS.

### BACKGROUND OF THE INVENTION

HIV-1 (human immunodeficiency virus-1) infection remains a major medical problem, with an estimated 45 million people infected worldwide at the end of 2007. The number of cases of HIV and AIDS (acquired immunodeficiency syndrome) has risen rapidly. In 2005, approximately 5.0 million new infections were reported, and 3.1 million people died from AIDS. Currently available drugs for the treatment of HIV include nucleoside reverse transcriptase (RT) inhibitors or approved single pill combinations: zidovudine (or AZT or RETROVIR®), didanosine (or VIDEX®), stavudine (or ZERIT®), lamivudine (or 3TC or EPIVIR®), zalcitabine (or DDC or HIVID®), abacavir succinate (or ZIAGEN®), tenofovir disoproxil fumarate salt (or VIREAD®), emtricitabine (or FTC-EMTRIVA®), COMBIVIR® (contains −3TC plus AZT), TRIZIVIR® (contains abacavir, lamivudine, and zidovudine), Epzicom (contains abacavir and lamivudine), TRUVADA® (contains VIREAD® and EMTRIVA®); non-nucleoside reverse transcriptase inhibitors: nevirapine (or VIRAMUNE®), delavirdine (or RESCRIPTOR®) and efavirenz (or SUSTIVA®), Atripla (TRUVADA®+SUSTIVA®), and etravirine, and peptidomimetic protease inhibitors or approved formulations: saquinavir, indinavir, ritonavir, nelfinavir, amprenavir, lopinavir, KALETRA® (lopinavir and Ritonavir), darunavir, atazanavir (REYATAZ®) and tipranavir (APTIVUS®), and integrase inhibitors such as raltegravir (Isentress), and entry inhibitors such as enfuvirtide (T-20) (FUZEON®) and maraviroc (Selzentry).

Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients may ultimately fail combination drug therapies. Insufficient drug potency, non-compliance, restricted tissue penetration and drug-specific limitations within certain cell types (e.g., most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present. Therefore, novel anti-HIV agents exhibiting distinct resistance patterns, and favorable pharmacokinetic as well as safety profiles are needed to provide more treatment options. Improved HIV fusion inhibitors and HIV entry coreceptor antagonists are two examples of new classes of anti-HIV agents further being studied by a number of investigators.

HIV attachment inhibitors are a novel subclass of antiviral compounds that bind to the HIV surface glycoprotein gp120, and interfere with the interaction between the surface protein gp120 and the host cell receptor CD4. Thus, they prevent HIV from attaching to the human CD4 T-cell, and block HIV replication in the first stage of the HIV life cycle. The properties of HIV attachment inhibitors have been improved in an effort to obtain compounds with maximized utility and efficacy as antiviral agents. A disclosure describing indoles of which the structure shown below for BMS-705 is representative, has been disclosed (Antiviral Indoleoxoacetyl piperazine Derivatives).

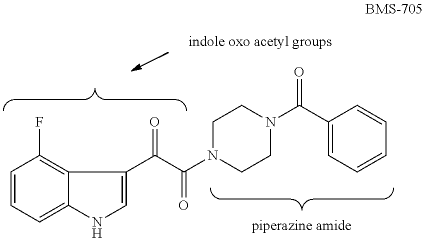 [SMILES: O=C(C(=O)N1CCN(C(=O)c2ccccc2)CC1)c1c[nH]c2cccc(F)c12]

Two other compounds, referred to in the literature as BMS-806 and BMS-043 have been described in both the academic and patent art:

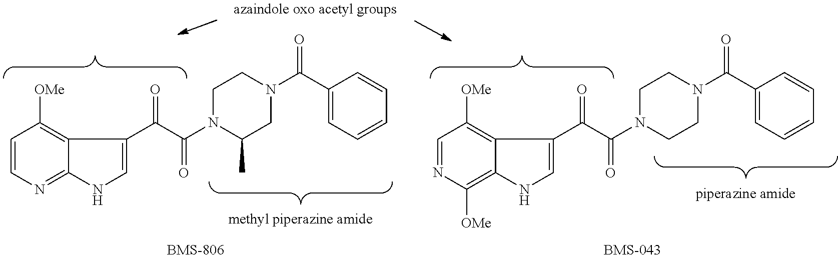 [SMILES: COc1ccnc2[nH]cc(C(=O)C(=O)N3CCN(C(=O)c4ccccc4)C[C@H]3C)c12 ; COc1ncc(OC)c2c(C(=O)C(=O)N3CCN(C(=O)c4ccccc4)CC3)c[nH]c12]

Some description of their properties in human clinical trials has been disclosed in the literature.

It should be noted that in all three of these structures, a piperazine amide (in these three structures a piperazine phenyl amide) is present and this group is directly attached to an oxoacetyl moiety. The oxoacetyl group is attached at the 3-position of 4-fluoro indole in BMS-705 and to the 3 position of substituted azaindoles in BMS-806 and BMS-043.

In an effort to obtain improved anti-HIV compounds, later publications described in part, modified substitution patterns on the indoles and azaindoles. Examples of such efforts include: (1) novel substituted indoleoxoacetic piperazine derivatives, (2) substituted piperazinyloxoacetylindole derivatives, and (3) substituted azaindoleoxoacetic piperazine derivatives.

Replacement of these groups with other heteroaromatics or substituted heteroaromatics or bicyclic hydrocarbons was also shown to be feasible. Examples include: (1) indole, azaindole and related heterocyclic amidopiperazine derivatives; (2) bicyclo 4.4.0 antiviral derivatives; and (3) diazaindole derivatives.

A select few replacements for the piperazine amide portion of the molecules have also been described in the art and among these examples are (1) some piperidine alkenes; (2) some pyrrolidine amides; (3) some N-aryl or heteroaryl piperazines; (4) some piperazinyl ureas; and (5) some carboline-containing compounds.

Method(s) for preparing prodrugs for this class of compounds are disclosed in Prodrugs of piperazine and Substituted Piperidine Antiviral Agents (Ueda et al., U.S. non-provisional application Ser. No. 11/066,745, filed Feb. 25, 2005 or U.S. Publication No. 2005/0209246 or WO 2005/090367 A1).

A published PCT patent application WO 2003/103607 A1 (Jun. 11, 2003) disclosures an assay useful for assaying some HIV inhibitors.

Several published patent applications describe combination studies with piperazine benzamide inhibitors, for example, U.S. Publication No. 2005/0215543 (WO 2005/102328 A1), U.S. Publication No. 2005/0215544 (WO 2005/102391 A1), and U.S. Publication No. 2005/0215545 (WO 2005/102392 A2).

A publication on new compounds in this class of attachment inhibitors (Wang, J. et al., Org. Biol. Chem., 3:1781-1786 (2005)) and a patent application on some more remotely related compounds have appeared WO 2005/016344 published on Feb. 24, 2005.

Published patent applications WO 2005/016344 and WO 2005/121094 also describe piperazine derivatives which are HIV inhibitors. Other references in the HIV attachment area include U.S. Publication Nos. 2007/0155702, 2007/0078141 and 2007/0287712, WO 2007/103456, as well as U.S. Pat. Nos. 7,348,337 and 7,354,924. A literature reference is J. Med. Chem., 50:6535 (2007).

What is therefore needed in the art are new HIV attachment inhibitor compounds, and compositions thereof, which are efficacious against HIV infection.

Of particular interest are new deuterated HIV attachment inhibitor compounds, hereinafter described, which are derived from the heavy isotope of hydrogen known as deuterium. Other companies such as Protia, LLC and Concert Pharmaceuticals have now published patent applications directed to deuterated analogs of certain compounds with potential to treat HIV. These include, by way of example, US 20090075942, US 20090076138, US 20090076097, WO 2009148600, WO 2009145852, and WO 2009055006. However, it is believed that these compounds are not structurally related to the compounds of the present invention.

### SUMMARY OF THE INVENTION

The present invention provides compounds of Formula I below, the pharmaceutically acceptable salts and/or solvates (e.g., hydrates) thereof, their pharmaceutical formulations, and their use in patients suffering from or susceptible to a virus such as HIV. The compounds of Formula I, their pharmaceutically acceptable salts and/or solvates are effective antiviral agents, particularly as inhibitors of HIV. They are useful for the treatment of HIV and AIDS.

One embodiment of the present invention is directed to a compound of Formula I, including pharmaceutically acceptable salts thereof:

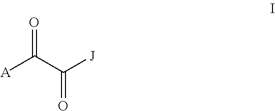

wherein A is selected from the group consisting of:

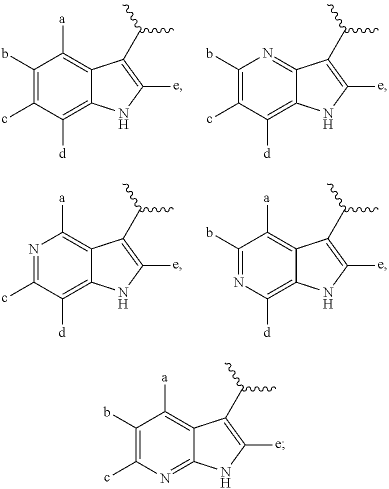

wherein
a, b, c, d and e are independently selected from the group consisting of hydrogen, halogen, cyano, nitro, COOR⁵⁶, XR⁵⁷, C(O)R⁷, C(O)NR⁵⁵R⁵⁶, B, Q, and E;
B is selected from the group consisting of —C(═NR⁴⁶)(R⁴⁷), C(O)NR⁴⁰R⁴¹, aryl, heteroaryl, heteroalicyclic, S(O)₂R⁸, C(O)R⁷, XR^{8a}, (C₁₋₆)alkylNR⁴⁰R⁴¹, (C₁₋₆)alkylCOOR^{8b}; wherein said aryl, heteroaryl, and heteroalicyclic are optionally substituted with one to three same or different halogens or from one to three same or different substituents selected from the group F; wherein aryl is napthyl or substituted phenyl; wherein heteroaryl is a mono or bicyclic system which contains from 3 to 7 ring atoms for a mono cyclic system and up to 12 atoms in a fused bicyclic system, including from 1 to 4 heteroatoms; wherein heteroalicyclic is a 3 to 7 membered mono cyclic ring which may contain from 1 to 2 heteroatoms in the ring skeleton and which may be fused to a benzene or pyridine ring;
Q is selected from the group consisting of (C₁₋₆)alkyl and (C₂₋₆)alkenyl; wherein said (C₁₋₆)alkyl and (C₂₋₆)alkenyl are optionally substituted with one to three same or different halogens or from one to three same or different substituents selected from the group consisting of C(O)NR⁵⁵R⁵⁶, hydroxy, cyano and XR⁵⁷;
E is selected from the group consisting of (C₁₋₆)alkyl and (C₂₋₆)alkenyl; wherein said (C₁₋₆)alkyl and (C₂₋₆)alkenyl are independently optionally substituted with a member selected from the group consisting of phenyl, heteroaryl, SMe, SPh,
—C(O)NR₅₆R₅₇, C(O)R₅₇, SO₂(C₁₋₆)alkyl and SO₂Ph; wherein heteroaryl is a monocyclic system which contains from 3 to 7 ring atoms, including from 1 to 4 heteroatoms;
F is selected from the group consisting of (C₁₋₆)alkyl, (C₃₋₇)cycloalkyl, aryl, heteroaryl, heteroalicyclic, hydroxy, (C₁₋₆)alkoxy, aryloxy, (C₁₋₆)thioalkoxy, cyano, halogen, nitro, —C(O)R⁵⁷, benzyl, —NR⁴²C(O)—(C₁₋₆)alkyl, —NR⁴²C(O)—
(C₃₋₆)cycloalkyl, —NR⁴²C(O)-aryl, —NR⁴²C(O)-heteroaryl, —NR⁴²C(O)-heteroalicyclic, a 4, 5, or 6 membered ring cyclic N-lactam, —NR⁴²S(O)₂—(C₁₋₆)alkyl, —NR⁴²S(O)₂—(C₃₋₆)cycloalkyl, —NR⁴²S(O)2-aryl, —NR⁴²S(O)₂-heteroaryl, —NR⁴²S(O)2-heteroalicyclic, S(O)₂(C₁₋₆)alkyl, S(O)₂aryl, —S(O)2NR⁴²R⁴³, NR⁴²R⁴³,
(C₁₋₆)alkylC(O)NR⁴²R⁴³, C(O)NR⁴²R⁴³, NHC(O)NR⁴²R⁴³, OC(O)NR⁴²R⁴³, NHC(O)OR⁵⁴, (C₁₋₆)alkylNR⁴²R⁴³, COOR⁵⁴, and (C₁₋₆)alkylCOOR⁵⁴; wherein said (C₁₋₆)alkyl, (C₃₋₇)cycloalkyl, aryl, heteroaryl, heteroalicyclic, (C₁₋₆)alkoxy, and aryloxy, are optionally substituted with one to nine same or different halogens or from one to five same or different substituents selected from the group G; wherein aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
G is selected from the group consisting of (C₁₋₆)alkyl, (C₃₋₇)cycloalkyl, aryl, heteroaryl, heteroalicyclic, hydroxy, (C₁₋₆)alkoxy, aryloxy, cyano, halogen, nitro,
—C(O)R⁵⁷, benzyl, —NR⁴⁸C(O)—(C₁₋₆)alkyl, —NR⁴⁸C(O)—(C₃₋₆)cycloalkyl, —NR⁴⁸C(O)-aryl, —NR⁴⁸C(O)-heteroaryl, —NR⁴⁸C(O)-heteroalicyclic, a 4, 5, or 6 membered ring cyclic N-lactam, —NR⁴⁸S(O)₂—(C₁₋₆)alkyl, —NR⁴⁸S(O)₂—
(C₃₋₆)cycloalkyl, —NR⁴⁸S(O)2-aryl, —NR⁴⁸S(O)₂-heteroaryl, —NR⁴⁸S(O)2-heteroalicyclic, sulfinyl, sulfonyl, sulfonamide, NR⁴⁸R⁴⁹, (C₁₋₆)alkyl C(O)NR⁴⁸R⁴⁹, C(O)NR⁴²R⁴⁹, NHC(O)NR⁴⁸R⁴⁹, OC(O)NR⁴⁸R⁴⁹, NHC(O)OR^{54′},
(C₁₋₆)alkylNR⁴⁸R⁴⁹, COOR⁵⁴, and (C₁₋₆)alkylCOOR⁵⁴; wherein
aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
R⁷ is selected from the group consisting of aryl, heteroaryl, and heteroalicyclic; wherein said aryl, heteroaryl, and heteroalicyclic are optionally substituted with one to three same or different halogens or with from one to three same or different substituents selected from the group F;
wherein for R⁷, R⁸, R^{8a}, R^{8b} aryl is phenyl; heteroaryl is a mono or bicyclic system which contains from 3 to 7 ring atoms for mono cyclic systems and up to 10 atoms in a bicyclic system, including from 1 to 4 heteroatoms; wherein heteroalicyclic is selected from the group consisting of aziridine, azetidine, pyrrolidine, piperazine, piperidine, tetrahydrofuran, tetrahydropyran, azepine, and morpholine;
R⁸ is selected from the group consisting of hydrogen, (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic; wherein said (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic are optionally substituted with one to six same or different halogens or from one to five same or different substituents selected from the group F;
R^{8a} is a member selected from the group consisting of aryl, heteroaryl, and heteroalicyclic; wherein each member is independently optionally substituted with one to six same or different halogens or from one to five same or different substituents selected from the group F;
R^{8b} is selected from the group consisting of hydrogen, (C₁₋₆)alkyl and phenyl; R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
X is selected from the group consisting of NH or NCH₃, O, and S;
R⁴⁰ and R⁴¹ are independently selected from the group consisting of
(a) hydrogen; (b) (C₁₋₆)alkyl or (C₃₋₇)cycloalkyl substituted with one to three same or different halogens or from one to two same or different substituents selected from the group F; and (c) (C₁₋₆)alkoxy, aryl, heteroaryl or heteroalicyclic; or R⁴⁰ and R⁴¹ taken together with the nitrogen to which they are attached form a member selected from the group consisting of aziridine, azetidine, pyrrolidine, piperazine, 4-NMe piperazine, piperidine, azepine, and morpholine; and wherein said aryl, heteroaryl, and heteroalicyclic are optionally substituted with one to three same or different halogens or from one to two same or different substituents selected from the group F; wherein for R⁴⁰ and R⁴¹ aryl is phenyl; heteroaryl is a monocyclic system which contains from 3 to 6 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine; provided when B is C(O)NR⁴⁰R⁴¹, at least one of R⁴⁰ and R⁴¹ is not selected from groups (a) or (b);
R⁴² and R⁴³ are independently selected from the group consisting of hydrogen, (C₁₋₆)alkyl, allyl, (C₁₋₆)alkoxy, (C₃₋₇)cycloalkyl, aryl, heteroaryl and heteroalicyclic; or R⁴² and R⁴³ taken together with the nitrogen to which they are attached form a member selected from the group consisting of aziridine, azetidine, pyrrolidine, piperazine, 4-NMe piperazine, piperidine, azepine, and morpholine; and wherein said (C₁₋₆)alkyl, (C₁₋₆)alkoxy, (C₃₋₇)cycloalkyl, aryl, heteroaryl, and heteroalicyclic are optionally substituted with one to three same or different halogens or from one to two same or different substituents selected from the group G; wherein for R⁴² and R⁴³ aryl is phenyl; heteroaryl is a monocyclic system which contains from 3 to 6 ring atoms, including from 1 to 4 heteroatoms; heteroalicyclic is a member selected from the group consisting of aziridine, azetidine, pyrrolidine, piperazine, piperidine, tetrahydrofuran, tetrahydropyran, azepine, and morpholine;
R⁴⁶ is selected from the group consisting of H, OR⁵⁷, and NR⁵⁵R⁵⁶;
R⁴⁷ is selected from the group consisting of H, amino, halogen, phenyl, and (C₁₋₆)alkyl;
R⁴⁸ and R⁴⁹ are independently selected from the group consisting of hydrogen, (C₁₋₆)alkyl and phenyl;
R⁵⁰ is selected from the group consisting of H, (C₁₋₆)alkyl, (C₃₋₆)cycloalkyl, and benzyl; wherein each of said (C₁₋₆)alkyl, (C₃₋₇)cycloalkyl and benzyl are optionally substituted with one to three same or different halogen, amino, OH, CN or NO₂;
R⁵⁴ is selected from the group consisting of hydrogen and (C₁₋₆)alkyl;
R^{54′} is (C₁₋₆)alkyl;
R⁵⁵ and R⁵⁶ are independently selected from the group consisting of hydrogen and (C₁₋₆)alkyl; and
R⁵⁷ is selected from the group consisting of hydrogen, (C₁₋₆)alkyl and phenyl; and
J is selected from the group consisting of:

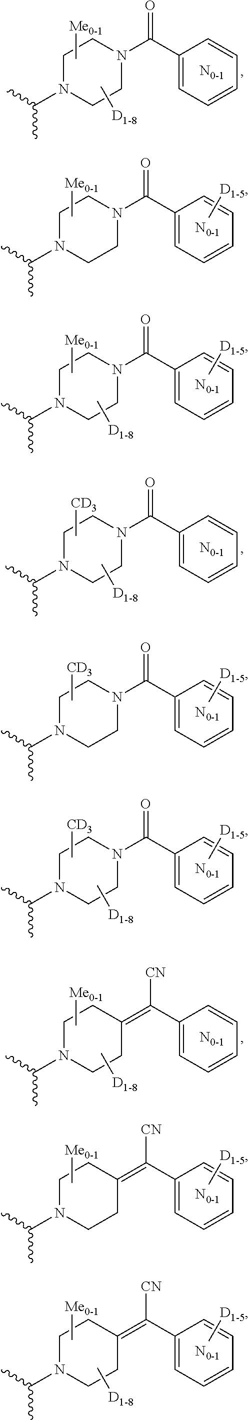

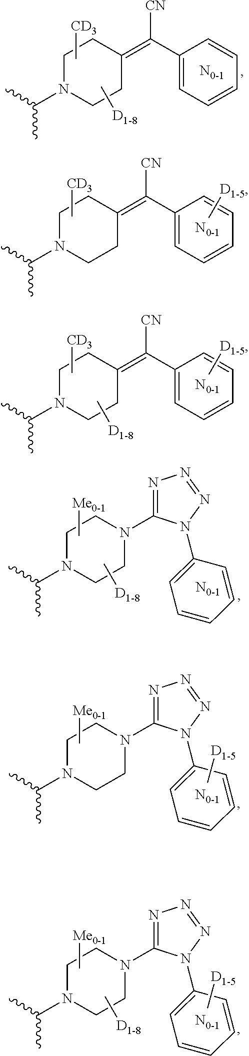

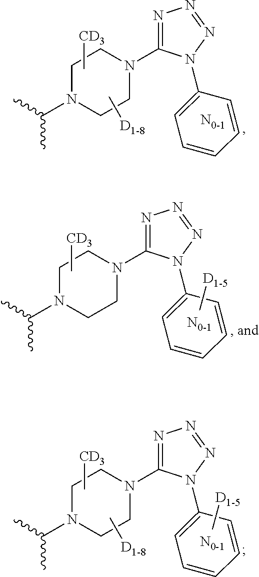

wherein Me represents methyl, and D represents deuterium.

Another embodiment of the present invention is directed to a method for treating mammals infected with a virus, especially wherein the virus is HIV, comprising administering to said mammal an antiviral effective amount of a compound of Formula I above, and one or more pharmaceutically acceptable carriers, excipients or diluents. Optionally, the compound of Formula I can be administered in combination with an antiviral effective amount of an AIDS treatment agent selected from the group consisting of: (a) an AIDS antiviral agent; (b) an anti-infective agent; (c) an immunomodulator; and (d) other HIV entry inhibitors.

Another embodiment of the present invention is a pharmaceutical composition comprising an antiviral effective amount of a compound of Formula I and one or more pharmaceutically acceptable carriers, excipients, diluents and optionally in combination with an antiviral effective amount of an AIDS treatment agent selected from the group consisting of: (a) an AIDS antiviral agent; (b) an anti-infective agent; (c) an immunomodulator; and (d) other HIV entry inhibitors.

In another embodiment of the invention there is provided one or more methods for making the compounds of Formula I.

The present invention is directed to these, as well as other important ends, hereinafter described.

### DETAILED DESCRIPTION OF THE EMBODIMENTS

Since the compounds of the present invention may possess asymmetric centers and therefore occur as mixtures of diastereomers and enantiomers, the present disclosure includes the individual diastereoisomeric and enantiomeric forms of the compounds of Formula I in addition to the mixtures thereof.

### DEFINITIONS

Unless otherwise specifically set forth elsewhere in the application, one or more of the following terms may be used herein, and shall have the following meanings:

The term “H” refers to hydrogen, including its isotopes.

The term “D” refers specifically to deuterium.

The term “C₁₋₆ alkyl” as used herein and in the claims (unless specified otherwise) mean straight or branched chain alkyl groups such as methyl, ethyl, propyl, isopropyl, butyl, isobutyl, t-butyl, amyl, hexyl and the like.

“C₁-C₄-fluoroalkyl” refers to F-substituted C₁-C₄ alkyl wherein at least one H atom is substituted with F atom, and each H atom can be independently substituted by F atom.

“Halogen” refers to chlorine, bromine, iodine or fluorine.

An “aryl” or “Ar” group refers to an all carbon monocyclic or fused-ring polycyclic (i.e., rings which share adjacent pairs of carbon atoms) groups having a completely conjugated pi-electron system. Examples, without limitation, of aryl groups are phenyl, napthalenyl and anthracenyl. The aryl group may be substituted or unsubstituted. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino and —NR^{x}R^{y}, wherein R^{x} and R^{y} are independently selected from the group consisting of hydrogen, alkyl, cycloalkyl, aryl, carbonyl, C-carboxy, sulfonyl, trihalomethyl, and, combined, a five- or six-member heteroalicyclic ring.

As used herein, a “heteroaryl” group refers to a monocyclic or fused ring (i.e., rings which share an adjacent pair of atoms) grouphaving in the ring(s) one or more atoms selected from the group consisting of nitrogen, oxygen and sulfur and, in addition, having a completely conjugated pi-electron system. Unless otherwise indicated, the heteroaryl group may be attached at either a carbon or nitrogen atom within the heteroaryl group. It should be noted that the term heteroaryl is intended to encompass an N-oxide of the parent heteroaryl if such an N-oxide is chemically feasible as is known in the art. Examples, without limitation, of heteroaryl groups are furyl, thienyl, benzothienyl, thiazolyl, imidazolyl, oxazolyl, oxadiazolyl, thiadiazolyl, benzothiazolyl, triazolyl, tetrazolyl, isoxazolyl, isothiazolyl, pyrrolyl, pyranyl, tetrahydropyranyl, pyrazolyl, pyridyl, pyrimidinyl, quinolinyl, isoquinolinyl, purinyl, carbazolyl, benzoxazolyl, benzimidazolyl, indolyl, isoindolyl, pyrazinyl. diazinyl, pyrazine, triazinyl, tetrazinyl, and tetrazolyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thioalkoxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino, and —NR^{x}R^{y}, wherein R^{x} and R^{y} are as defined above.

As used herein, a “heteroalicyclic” group refers to a monocyclic or fused ring grouphaving in the ring(s) one or more atoms selected from the group consisting of nitrogen, oxygen and sulfur. Rings are selected from those which provide stable arrangements of bonds and are not intended to encompass systems which would not exist. The rings may also have one or more double bonds. However, the rings do not have a completely conjugated pi-electron system. Examples, without limitation, of heteroalicyclic groups are azetidinyl, piperidyl, piperazinyl, imidazolinyl, thiazolidinyl, 3-pyrrolidin-1-yl, morpholinyl, thiomorpholinyl and tetrahydropyranyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halogen, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, silyl, guanyl, guanidino, ureido, phosphonyl, amino and —NR^{x}R^{y}, wherein R^{x} and R^{y} are as defined above.

An “alkyl” group refers to a saturated aliphatic hydrocarbon including straight chain and branched chain groups. Preferably, the alkyl grouphas 1 to 20 carbon atoms (whenever a numerical range; e.g., “1-20”, is stated herein, it means that the group, in this case the alkyl group may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The alkyl group may be substituted or unsubstituted. When substituted, the substituent group(s) is preferably one or more individually selected from trihaloalkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, and combined, a five- or six-member heteroalicyclic ring.

A “cycloalkyl” group refers to an all-carbon monocyclic or fused ring (i.e., rings which share and adjacent pair of carbon atoms) group wherein one or more rings does not have a completely conjugated pi-electron system. Examples, without limitation, of cycloalkyl groups are cyclopropane, cyclobutane, cyclopentane, cyclopentene, cyclohexane, cyclohexene, cycloheptane, cycloheptene and adamantane. A cycloalkyl group may be substituted or unsubstituted. When substituted, the substituent group(s) is preferably one or more individually selected from alkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, silyl, guanyl, guanidino, ureido, phosphonyl, amino and —NR^{x}R^{y} with R^{x} and R^{y} as defined above.

An “alkenyl” group refers to an alkyl group, as defined herein, having at least two carbon atoms and at least one carbon-carbon double bond.

An “alkynyl” group refers to an alkyl group, as defined herein, having at least two carbon atoms and at least one carbon-carbon triple bond.

A “hydroxy” group refers to an —OH group.

An “alkoxy” group refers to both an —O-alkyl and an —O-cycloalkyl group as defined herein.

An “aryloxy” group refers to both an —O-aryl and an —O-heteroaryl group, as defined herein.

A “heteroaryloxy” group refers to a heteroaryl-O— group with heteroaryl as defined herein.

A “heteroalicycloxy” group refers to a heteroalicyclic-O— group with heteroalicyclic as defined herein.

A “thiohydroxy” group refers to an —SH group.

A “thioalkoxy” group refers to both an S-alkyl and an —S-cycloalkyl group, as defined herein.

A “thioaryloxy” group refers to both an —S-aryl and an —S-heteroaryl group, as defined herein.

A “thioheteroaryloxy” group refers to a heteroaryl-S— group with heteroaryl as defined herein.

A “thioheteroalicycloxy” group refers to a heteroalicyclic-S— group with heteroalicyclic as defined herein.

A “carbonyl” group refers to a —C(═O)—R″ group, where R″ is selected from the group consisting of hydrogen, alkyl, alkenyl, alkynyl, cycloalkyl, aryl, heteroaryl (bonded through a ring carbon) and heteroalicyclic (bonded through a ring carbon), as each is defined herein.

An “aldehyde” group refers to a carbonyl group where R″ is hydrogen.

A “thiocarbonyl” group refers to a —C(═S)—R″ group, with R″ as defined herein.

A “Keto” group refers to a —CC(═O)C— group wherein the carbon on either or both sides of the C═O may be alkyl, cycloalkyl, aryl or a carbon of a heteroaryl or heteroalicyclic group.

A “trihalomethanecarbonyl” group refers to a Z₃CC(═O)— group with said Z being a halogen.

A “C-carboxy” group refers to a —C(═O)O—R″ groups, with R″ as defined herein.

An “O-carboxy” group refers to a R″C(—O)O— group, with R″ as defined herein.

A “carboxylic acid” group refers to a C-carboxy group in which R″ is hydrogen.

A “trihalomethyl” group refers to a —CZ₃, group wherein Z is a halogen group as defined herein.

A “trihalomethanesulfonyl” group refers to an Z₃CS(═O)₂— groups with Z as defined above.

A “trihalomethanesulfonamido” group refers to a Z₃CS(═O)₂NR^{x}— group with Z as defined above and R^{x} being H or (C₁₋₆)alkyl.

A “sulfinyl” group refers to a —S(═O)—R″ group, with R″ being (C₁₋₆)alkyl.

A “sulfonyl” group refers to a —S(═O)₂R″ group with R″ being (C₁₋₆)alkyl.

A “S-sulfonamido” group refers to a —S(═O)₂NR^{X}R^{Y}, with R^{X} and R^{Y} independently being H or (C₁₋₆)alkyl.

A “N-Sulfonamido” group refers to a R″S(═O)₂NR_{X}— group, with Rₓ being H or (C₁₋₆)alkyl.

A “O-carbamyl” group refers to a —OC(═O)NR^{x}R^{y} group, with R^{X} and R^{Y} independently being H or (C₁₋₆)alkyl.

A “N-carbamyl” group refers to a R^{x}OC(═O)NR^{y} group, with R^{x} and R^{y} independently being H or (C₁₋₆)alkyl.

A “O-thiocarbamyl” group refers to a —OC(═S)NR^{x}R^{y} group, with R^{x} and R^{y} independently being H or (C₁₋₆)alkyl.

A “N-thiocarbamyl” group refers to a R^{x}OC(═S)NR^{y}— group, with R^{x} and R^{y} independently being H or (C₁₋₆)alkyl.

An “amino” group refers to an —NH₂ group.

A “C-amido” group refers to a —C(═O)NR^{x}R^{y} group, with R^{x} and R^{y} independently being H or (C₁₋₆)alkyl.

A “C-thioamido” group refers to a —C(═S)NR^{x}R^{y} group, with R^{x} and R^{y} independently being H or (C₁₋₆)alkyl.

A “N-amido” group refers to a R^{x}C(═O)NR^{y}— group, with R^{x} and R^{y} independently being H or (C₁₋₆)alkyl.

An “ureido” group refers to a —NR^{x}C(═O)NR^{y}R^{y2} group, with R^{x}, R^{y}, and R^{y2} independently being H or (C₁₋₆)alkyl.

A “guanidino” group refers to a —R^{x}NC(═N)NR^{y}R^{y2} group, with R^{x}, R^{y}, and R^{y2} independently being H or (C₁₋₆)alkyl.

A “guanyl” group refers to a R^{x}R^{y}NC(═N)— group, with R^{x} and R^{y} independently being H or (C₁₋₆)alkyl.

A “cyano” group refers to a —CN group.

A “silyl” group refers to a —Si(R″)₃, with R″ being (C₁₋₆)alkyl or phenyl.

A “phosphonyl” group refers to a P(═O)(OR^{x})₂ with R^{x} being (C₁₋₆)alkyl.

A “hydrazino” group refers to a —NR^{x}NR^{y}R^{y2} group, with R^{x}, R^{y}, and R^{y2} independently being H or (C₁₋₆)alkyl.

A “4, 5, or 6 membered ring cyclic N-lactam” group refers to

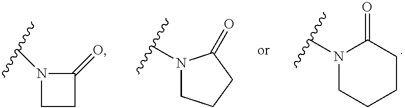 [SMILES: *N1CCC1=O ; *N1CCCC1=O ; "or" ; *N1CCCCC1=O]

Any two adjacent R groups may combine to form an additional aryl, cycloalkyl, heteroaryl or heterocyclic ring fused to the ring initially bearing those R groups.

It is known in the art that nitrogen atoms in heteroaryl systems can be “participating in a heteroaryl ring double bond”, and this refers to the form of double bonds in the two tautomeric structures which comprise five-member ring heteroaryl groups. This dictates whether nitrogens can be substituted as well understood by chemists in the art. The disclosure and claims of the present disclosure are based on the known general principles of chemical bonding. It is understood that the claims do not encompass structures known to be unstable or not able to exist based on the literature.

Pharmaceutically acceptable salts and prodrugs of compounds disclosed herein are within the scope of this disclosure. The term “pharmaceutically acceptable salt” as used herein and in the claims is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfuric acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g., hydroxyl-substituted alkylamines such as diethanolamine, triethanolamine or tris(hydroxymethyl)-aminomethane), or with bases such as piperidine or morpholine.

As stated above, the compounds of the invention also include “prodrugs”. The term “prodrug” as used herein encompasses both the term “prodrug esters” and the term “prodrug ethers”. The term “prodrug esters” as employed herein includes esters and carbonates formed by reacting one or more hydroxyls of compounds of Formula I with either alkyl, alkoxy, or aryl substituted acylating agents or phosphorylating agent employing procedures known to those skilled in the art to generate acetates, pivalates, methylcarbonates, benzoates, amino acid esters, phosphates, half acid esters such as malonates, succinates or glutarates, and the like. In certain embodiments, amino acid esters may be especially preferred.

Examples of such prodrug esters include

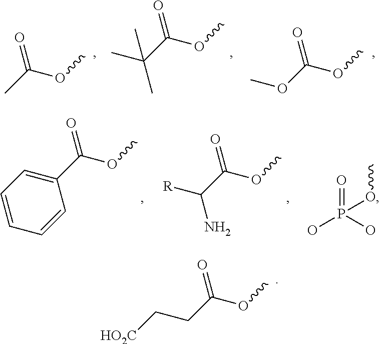

The term “prodrug ethers” include both phosphate acetals and O-glucosides. Representative examples of such prodrug ethers include

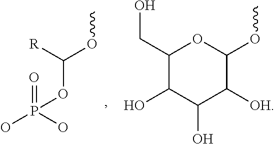

Prodrug derivatives in which the prodrug moiety is attached to the indole N atom are also considered part of this invention. These prodrugs can be prepared by substitution of the indole N with a moiety that modifies the physical properties of the compound and can be unmasked either by chemical or enzymatic degradation. Examples of R₃ include acyl derivatives similar to those described above. A preferred prodrug is the phosphonoxymethyl moiety which can be introduced using methods previously described and converted to pharmaceutically acceptable salt forms that confer chemical stability and advantageous physical properties:

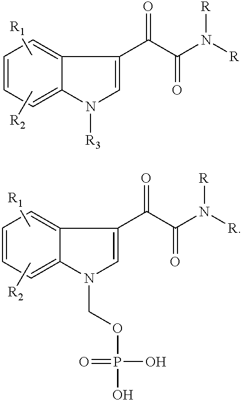

As set forth above, the invention is directed to compounds of Formula I, including pharmaceutically acceptable salts thereof:

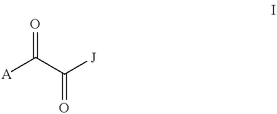

wherein A is selected from the group consisting of:

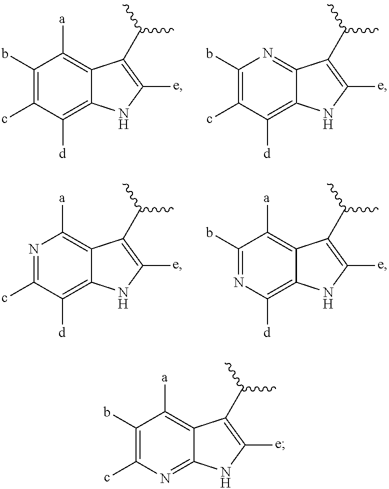

wherein
a, b, c, d and e are independently selected from the group consisting of hydrogen, halogen, cyano, nitro, COOR⁵⁶, XR⁵⁷, C(O)R⁷, C(O)NR⁵⁵R⁵⁶, B, Q, and E;
B is selected from the group consisting of —C(═NR⁴⁶)(R⁴⁷), C(O)NR⁴⁰R⁴¹, aryl, heteroaryl, heteroalicyclic, S(O)₂R⁸, C(O)R⁷, XR^{8a}, (C₁₋₆)alkylNR⁴⁰R⁴¹, (C₁₋₆)alkylCOOR^{8b}; wherein said aryl, heteroaryl, and heteroalicyclic are optionally substituted with one to three same or different halogens or from one to three same or different substituents selected from the group F; wherein aryl is napthyl or substituted phenyl; wherein heteroaryl is a mono or bicyclic system which contains from 3 to 7 ring atoms for a mono cyclic system and up to 12 atoms in a fused bicyclic system, including from 1 to 4 heteroatoms; wherein heteroalicyclic is a 3 to 7 membered mono cyclic ring which may contain from 1 to 2 heteroatoms in the ring skeleton and which may be fused to a benzene or pyridine ring;
Q is selected from the group consisting of (C₁₋₆)alkyl and (C₂₋₆)alkenyl; wherein said (C₁₋₆)alkyl and (C₂₋₆)alkenyl are optionally substituted with one to three same or different halogens or from one to three same or different substituents selected from the group consisting of C(O)NR⁵⁵R⁵⁶, hydroxy, cyano and XR⁵⁷;

E is selected from the group consisting of (C₁₋₆)alkyl and (C₂₋₆)alkenyl; wherein said (C₁₋₆)alkyl and (C₂₋₆)alkenyl are independently optionally substituted with a member selected from the group consisting of phenyl, heteroaryl, SMe, SPh,

—C(O)NR₅₆R₅₇, C(O)R₅₇, SO₂(C₁₋₆)alkyl and SO₂Ph; wherein heteroaryl is a monocyclic system which contains from 3 to 7 ring atoms, including from 1 to 4 heteroatoms;
F is selected from the group consisting of (C₁₋₆)alkyl, (C₃₋₇)cycloalkyl, aryl, heteroaryl, heteroalicyclic, hydroxy, (C₁₋₆)alkoxy, aryloxy, (C₁₋₆)thioalkoxy, cyano, halogen, nitro, —C(O)R⁵⁷, benzyl, —NR⁴²C(O)—(C₁₋₆)alkyl, —NR⁴²C(O)—
(C₃₋₆)cycloalkyl, —NR⁴²C(O)-aryl, —NR⁴²C(O)-heteroaryl, —NR⁴²C(O)-heteroalicyclic, a 4, 5, or 6 membered ring cyclic N-lactam, —NR⁴²S(O)₂—(C₁₋₆)alkyl, —NR⁴²S(O)₂—(C₃₋₆)cycloalkyl, —NR⁴²S(O)₂-aryl, —NR⁴²S(O)₂-heteroaryl, —NR⁴²S(O)₂-heteroalicyclic, S(O)₂(C₁₋₆)alkyl, S(O)₂aryl, —S(O)2 NR⁴²R⁴³, NR⁴²R⁴³,
(C₁₋₆)alkylC(O)NR⁴²R⁴³, C(O)NR⁴²R⁴³, NHC(O)NR⁴²R⁴³, OC(O)NR⁴²R⁴³, NHC(O)OR⁵⁴, (C₁₋₆)alkylNR⁴²R⁴³, COOR⁵⁴, and (C₁₋₆)alkylCOOR⁵⁴; wherein said (C₁₋₆)alkyl, (C₃₋₇)cycloalkyl, aryl, heteroaryl, heteroalicyclic, (C₁₋₆)alkoxy, and aryloxy, are optionally substituted with one to nine same or different halogens or from one to five same or different substituents selected from the group G; wherein aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
G is selected from the group consisting of (C₁₋₆)alkyl, (C₃₋₇)cycloalkyl, aryl, heteroaryl, heteroalicyclic, hydroxy, (C₁₋₆)alkoxy, aryloxy, cyano, halogen, nitro,
—C(O)R⁵⁷, benzyl, —NR⁴⁸C(O)—(C₁₋₆)alkyl, —NR⁴⁸C(O)—(C₃₋₆)cycloalkyl, —NR⁴⁸C(O)-aryl, —NR⁴⁸C(O)-heteroaryl, —NR⁴⁸C(O)-heteroalicyclic, a 4, 5, or 6 membered ring cyclic N-lactam, —NR⁴⁸S(O)₂—(C₁₋₆)alkyl, —NR⁴⁸S(O)₂—
(C₃₋₆)cycloalkyl, —NR⁴⁸S(O)₂-aryl, —NR⁴⁸S(O)₂-heteroaryl, —NR⁴⁸S(O)₂-heteroalicyclic, sulfinyl, sulfonyl, sulfonamide, NR⁴⁸R⁴⁹, (C₁₋₆)alkyl C(O)NR⁴⁸R⁴⁹, C(O)NR⁴⁸R⁴⁹, NHC(O)NR⁴⁸R⁴⁹, OC(O)NR⁴⁸R⁴⁹, NHC(O)OR^{54′},
(C₁₋₆)alkylNR⁴⁸R⁴⁹, COOR⁵⁴, and (C₁₋₆)alkylCOOR⁵⁴; wherein
aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
R⁷ is selected from the group consisting of aryl, heteroaryl, and heteroalicyclic; wherein said aryl, heteroaryl, and heteroalicyclic are optionally substituted with one to three same or different halogens or with from one to three same or different substituents selected from the group F;
wherein for R⁷, R⁸, R^{8a}, R^{8b} aryl is phenyl; heteroaryl is a mono or bicyclic system which contains from 3 to 7 ring atoms for mono cyclic systems and up to 10 atoms in a bicyclic system, including from 1 to 4 heteroatoms; wherein heteroalicyclic is selected from the group consisting of aziridine, azetidine, pyrrolidine, piperazine, piperidine, tetrahydrofuran, tetrahydropyran, azepine, and morpholine;
R⁸ is selected from the group consisting of hydrogen, (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic; wherein said (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic are optionally substituted with one to six same or different halogens or from one to five same or different substituents selected from the group F;
R^{8a} is a member selected from the group consisting of aryl, heteroaryl, and heteroalicyclic; wherein each member is independently optionally substituted with one to six same or different halogens or from one to five same or different substituents selected from the group F;
R^{8b} is selected from the group consisting of hydrogen, (C₁₋₆)alkyl and phenyl; R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
X is selected from the group consisting of NH or NCH₃, O, and S;
R⁴⁰ and R⁴¹ are independently selected from the group consisting of
(a) hydrogen; (b) (C₁₋₆)alkyl or (C₃₋₇)cycloalkyl substituted with one to three same or different halogens or from one to two same or different substituents selected from the group F; and (c) (C₁₋₆)alkoxy, aryl, heteroaryl or heteroalicyclic; or R⁴⁰ and R⁴¹ taken together with the nitrogen to which they are attached form a member selected from the group consisting of aziridine, azetidine, pyrrolidine, piperazine, 4-NMe piperazine, piperidine, azepine, and morpholine; and wherein said aryl, heteroaryl, and heteroalicyclic are optionally substituted with one to three same or different halogens or from one to two same or different substituents selected from the group F; wherein for R⁴⁰ and R⁴¹ aryl is phenyl; heteroaryl is a monocyclic system which contains from 3 to 6 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine; provided when B is C(O)NR⁴⁰R⁴¹, at least one of R⁴⁰ and R⁴¹ is not selected from groups (a) or (b);
R⁴² and R⁴³ are independently selected from the group consisting of hydrogen, (C₁₋₆)alkyl, allyl, (C₁₋₆)alkoxy, (C₃₋₇)cycloalkyl, aryl, heteroaryl and heteroalicyclic; or R⁴² and R⁴³ taken together with the nitrogen to which they are attached form a member selected from the group consisting of aziridine, azetidine, pyrrolidine, piperazine, 4-NMe piperazine, piperidine, azepine, and morpholine; and wherein said (C₁₋₆)alkyl, (C₁₋₆)alkoxy, (C₃₋₇)cycloalkyl, aryl, heteroaryl, and heteroalicyclic are optionally substituted with one to three same or different halogens or from one to two same or different substituents selected from the group G; wherein for R⁴² and R⁴³ aryl is phenyl; heteroaryl is a monocyclic system which contains from 3 to 6 ring atoms, including from 1 to 4 heteroatoms; heteroalicyclic is a member selected from the group consisting of aziridine, azetidine, pyrrolidine, piperazine, piperidine, tetrahydrofuran, tetrahydropyran, azepine, and morpholine;
R⁴⁶ is selected from the group consisting of H, OR⁵⁷, and NR⁵⁵R⁵⁶;
R⁴⁷ is selected from the group consisting of H, amino, halogen, phenyl, and (C₁₋₆)alkyl;
R⁴⁸ and R⁴⁹ are independently selected from the group consisting of hydrogen, (C₁₋₆)alkyl and phenyl;
R⁵⁰ is selected from the group consisting of H, (C₁₋₆)alkyl, (C₃₋₆)cycloalkyl, and benzyl; wherein each of said (C₁₋₆)alkyl, (C₃₋₇)cycloalkyl and benzyl are optionally substituted with one to three same or different halogen, amino, OH, CN or NO₂;
R⁵⁴ is selected from the group consisting of hydrogen and (C₁₋₆)alkyl;
R^{54′} is (C₁₋₆)alkyl;
R⁵⁵ and R⁵⁶ are independently selected from the group consisting of hydrogen and (C₁₋₆)alkyl; and
R⁵⁷ is selected from the group consisting of hydrogen, (C₁₋₆)alkyl and phenyl; and
J is selected from the group consisting of:

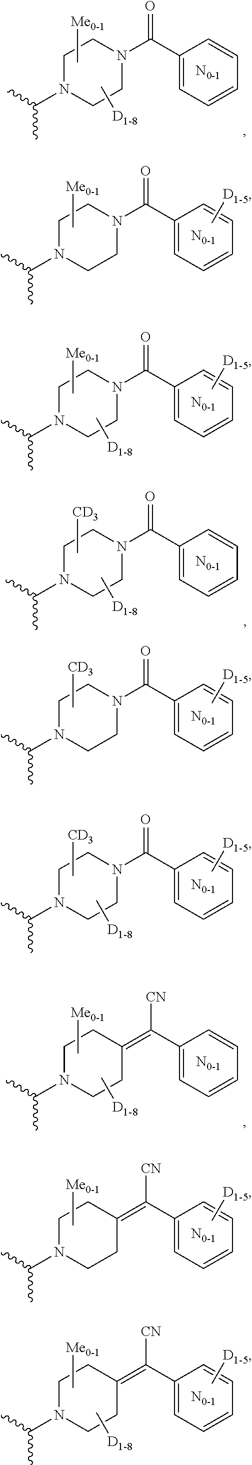

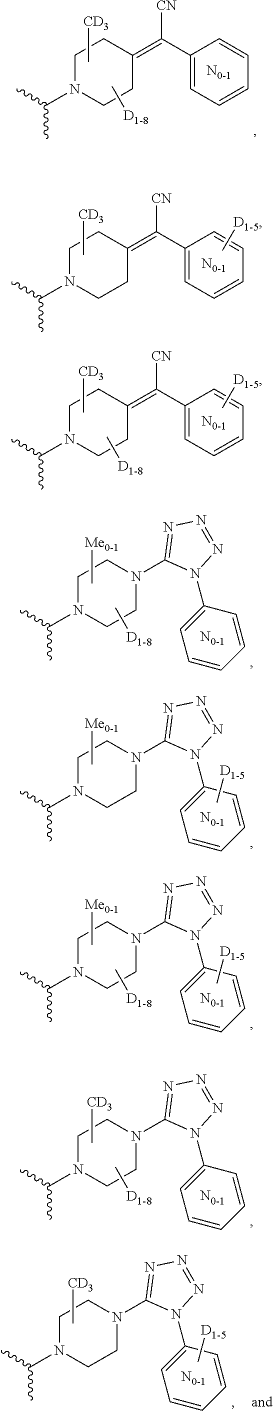

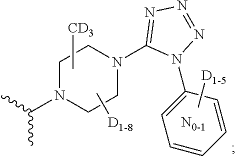

wherein Me represents methyl, and D represents deuterium.

In a further embodiment of Formula I above, there is the proviso that at least one of a-e is selected from B or E.

More preferred compounds of Formula I include those which are selected from the group consisting of:

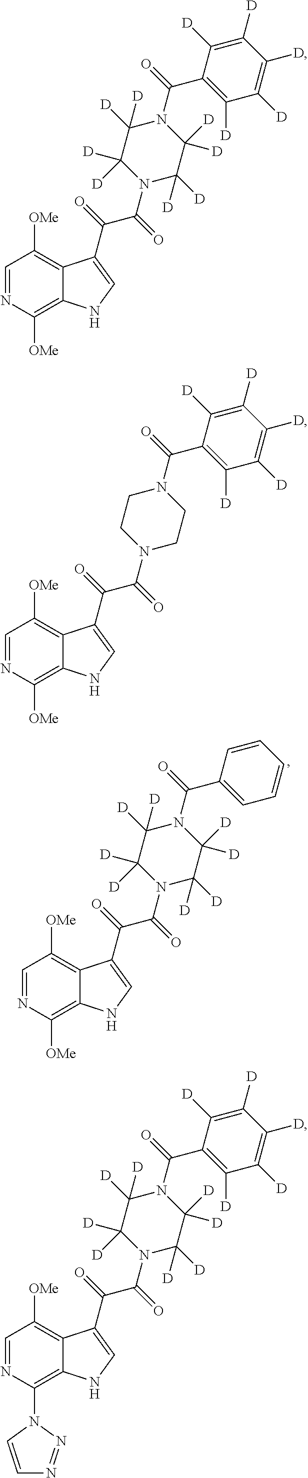

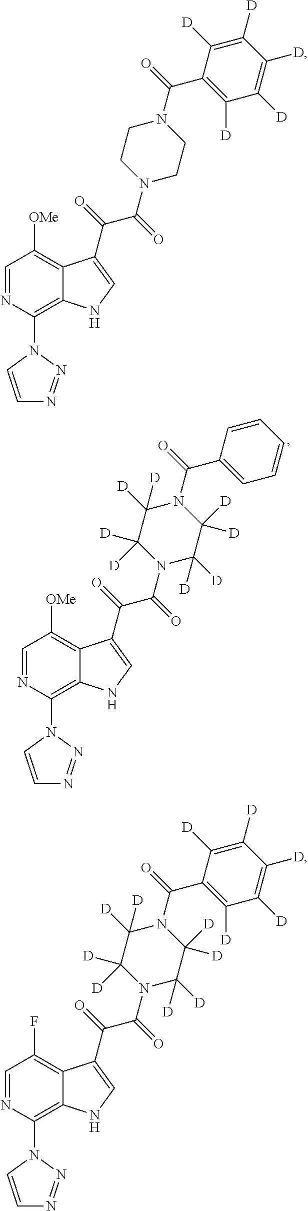

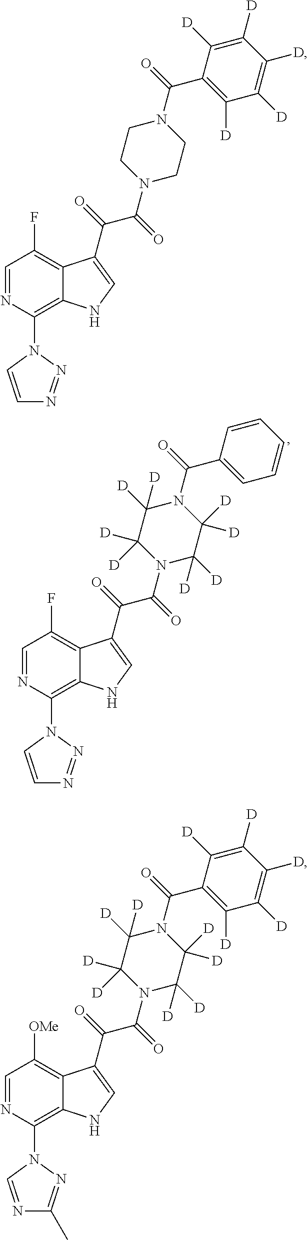

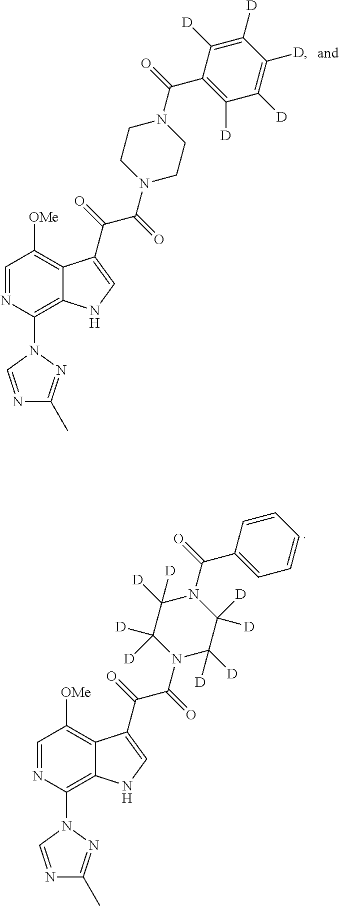

The compounds of the present invention, according to all the various embodiments described above, may be administered orally, parenterally (including subcutaneous injections, intravenous, intramuscular, intrasternal injection or infusion techniques), by inhalation spray, or rectally, and by other means, in dosage unit formulations containing non-toxic pharmaceutically acceptable carriers, excipients and diluents available to the skilled artisan. One or more adjuvants may also be included.

Thus, in accordance with the present disclosure, there is further provided a method of treatment, and a pharmaceutical composition, for treating viral infections such as HIV infection and AIDS. The treatment involves administering to a patient in need of such treatment a pharmaceutical composition which contains an antiviral effective amount of one or more of the compounds of Formula I, together with one or more pharmaceutically acceptable carriers, excipients or diluents. As used herein, the term “antiviral effective amount” means the total amount of each active component of the composition and method that is sufficient to show a meaningful patient benefit, i.e., inhibiting, ameliorating, or healing of acute conditions characterized by inhibition of the HIV infection. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “treat, treating, treatment” as used herein and in the claims means preventing, ameliorating or healing diseases associated with HIV infection.

The pharmaceutical compositions of the invention may be in the form of orally administrable suspensions or tablets; as well as nasal sprays, sterile injectable preparations, for example, as sterile injectable aqueous or oleaginous suspensions or suppositories. Pharmaceutically acceptable carriers, excipients or diluents may be utilized in the pharmaceutical compositions, and are those utilized in the art of pharmaceutical preparations.

When administered orally as a suspension, these compositions are prepared according to techniques typically known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art.

The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The compounds of this disclosure can be administered orally to humans in a dosage range of 1 to 100 mg/kg body weight in divided doses, usually over an extended period, such as days, weeks, months, or even years. One preferred dosage range is 1 to 10 mg/kg body weight orally in divided doses. Another preferred dosage range is 1 to 20 mg/kg body weight in divided doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also contemplated herein are combinations of the compounds of Formula I herein set forth, together with one or more agents useful in the treatment of AIDS. For example, the compounds of this disclosure may be effectively administered, whether at periods of pre-exposure and/or post-exposure, in combination with effective amounts of the AIDS antivirals, immunomodulators, anti-infectives, or vaccines, such as those in the following non-limiting table:

### Antivirals

| Drug Name | Manufacturer | Indication |
|---|---|---|
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| BMS-234475 | Bristol-Myers Squibb/ | HIV infection, |
| (CGP-61755) | Novartis | AIDS, ARC |
| | | (protease inhibitor) |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, Sustiva ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (Viread ®) | | AIDS, |
| | | (reverse transcriptase |
| | | inhibitor) |
| Emtriva ® (Emtricitabine) | Gilead | HIV infection, |
| (FTC) | | AIDS, |
| | | (reverse transcriptase |
| | | inhibitor) |
| Combivir ® | GSK | HIV infection, |
| | | AIDS, |
| | | (reverse transcriptase |
| | | inhibitor) |
| Abacavir succinate | GSK | HIV infection, |
| (or Ziagen ®) | | AIDS, |
| | | (reverse transcriptase |
| | | inhibitor) |
| Reyataz ® | Bristol-Myers Squibb | HIV infection |
| (or atazanavir) | | AIDs, protease |
| | | inhibitor |
| Fuzeon ® | Roche/Trimeris | HIV infection |
| (Enfuvirtide or T-20) | | AIDs, viral Fusion |
| | | inhibitor |
| Lexiva ® | GSK/Vertex | HIV infection |
| (or Fosamprenavir calcium) | | AIDs, viral protease |
| | | inhibitor |
| Selzentry | | |
| Maraviroc; (UK 427857) | Pfizer | HIV infection |
| | | AIDs, (CCR5 antagonist, in |
| | | development) |
| Trizivir ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| Truvada ® | Gilead | Combination of Tenofovir |
| | | disoproxil fumarate salt |
| | | (Viread ®) and Emtriva ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| Atripla ® | | disoproxil fumarate salt |
| | | (Viread ®), Emtriva ® |
| | | (Emtricitabine), and |
| | | Sustiva ® (Efavirenz) |
| Festinavir ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |

### Immunomodulators

| Drug Name | Manufacturer | Indication |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

### Anti-Infectives

| Drug Name | Manufacturer | Indication |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

Additionally, the compounds of the disclosure herein set forth may be used in combination with other HIV entry inhibitors. Examples of such HIV entry inhibitors are discussed in Drugs of the Future, 24(12):1355-1362 (1999); Cell, 9:243-246 (Oct. 29, 1999); and Drug Discovery Today, 5(5):183-194 (May 2000) and Meanwell, N. A. et al., “Inhibitors of the entry of HIV into host cells”, Curr. Op. Drug Disc. Dev, 6(4):451-461 (2003). Specifically the compounds can be utilized in combination with other attachment inhibitors, fusion inhibitors, and chemokine receptor antagonists aimed at either the CCR5 or CXCR4 coreceptor.

It will be understood that the scope of combinations of the compounds of this disclosure with AIDS antivirals, immunomodulators, anti-infectives, HIV entry inhibitors or vaccines is not limited to the list in the above Table but includes, in principle, any combination with any pharmaceutical composition useful for the treatment of AIDS.

Preferred combinations are simultaneous or alternating treatments with a compound of the present disclosure and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddI. A preferred inhibitor of HIV protease is REYATAZ® (active ingredient Atazanavir). Typically a dose of 300 to 600 mg is administered once a day. This may be co-administered with a low dose of Ritonavir (50 to 500 mgs). Another preferred inhibitor of HIV protease is KALETRA®. Another useful inhibitor of HIV protease is indinavir, which is the sulfate salt of N-(2(R)-hydroxy-1-(S)-indanyl)-2(R)-phenylmethyl-4-(S)-hydroxy-5-(1-(4-(3-pyridyl-methyl)-2(S)—N′-(t-butylcarboxamido)-piperazinyl))-pentaneamide ethanolate, and is synthesized according to U.S. Pat. No. 5,413,999. Indinavir is generally administered at a dosage of 800 mg three times a day. Other preferred protease inhibitors are nelfinavir and ritonavir. Another preferred inhibitor of HIV protease is saquinavir which is administered in a dosage of 600 or 1200 mg tid. Preferred non-nucleoside inhibitors of HIV reverse transcriptase include efavirenz. These combinations may have unexpected effects on limiting the spread and degree of infection of HIV. Preferred combinations include those with the following (1) indinavir with efavirenz, and, optionally, AZT and/or 3TC and/or ddI and/or ddC; (2) indinavir, and any of AZT and/or ddI and/or ddC and/or 3TC, in particular, indinavir and AZT and 3TC; (3) stavudine and 3TC and/or zidovudine; (4) zidovudine and lamivudine and 141W94 and 1592U89; (5) zidovudine and lamivudine. (The preparation of ddC, ddI and AZT are also described in EP 0 484 071.)

In such combinations the compound of the present disclosure and other active agents may be administered separately or in conjunction. In addition, the administration of one element may be prior to, concurrent to, or subsequent to the administration of other agent(s).

### General Chemistry (Methods of Synthesis)

The present invention comprises compounds of Formula I, their pharmaceutical formulations, and their use in patients suffering from or susceptible to HIV infection. The compounds of Formula I include pharmaceutically acceptable salts thereof. General procedures to construct compounds of Formula I and intermediates useful for their synthesis are described in the following Schemes (after the Abbreviations).

### Abbreviations

One or more of the following abbreviations, most of which are conventional abbreviations well known to those skilled in the art, may be used throughout the description of the disclosure and the examples:

h=hour(s)
rt=room temperature
mol=mole(s)
mmol=millimole(s)
g=gram(s)
mg=milligram(s)
mL=milliliter(s)
TFA=trifluoroacetic Acid

### DCE=1,2-Dichloroethane

CH₂Cl₂=dichloromethane
TPAP=tetrapropylammonium perruthenate
THF=tetrahydrofuran
DEPBT=3-(diethoxyphosphoryloxy)-1,2,3-benzotriazin-4(3H)-one
DMAP=4-dimethylaminopyridine
P-EDC=polymer supported 1-(3-dimethylaminopropyl)-3-ethylcarbodiimide
EDC=1-(3-dimethylaminopropyl)-3-ethylcarbodiimide

### DMF=N,N-dimethylformamide

### Hunig's Base=N,N-diisopropylethylamine

MCPBA=meta-chloroperbenzoic acid
azaindole=1H-pyrrolo-pyridine
4-azaindole=1H-pyrrolo[3,2-b]pyridine
5-azaindole=1H-pyrrolo[3,2-c]pyridine
6-azaindole=1H-pyrrolo[2,3-c]pyridine
7-azaindole=1H-pyrrolo[2,3-b]pyridine
PMB=4-methoxybenzyl
DDQ=2,3-dichloro-5,6-dicyano-1,4-benzoquinone
OTf=trifluoromethanesulfonoxy
NMM=4-methylmorpholine
PIP-COPh=1-benzoylpiperazine
NaHMDS=sodium hexamethyldisilazide
EDAC=1-(3-dimethylaminopropyl)-3-ethylcarbodiimide
TMS=trimethylsilyl
DCM=dichloromethane
DCE=dichloroethane
MeOH=methanol
THF=tetrahydrofuran
EtOAc=ethyl acetate
LDA=lithium diisopropylamide
TMP-Li=2,2,6,6-tetramethylpiperidinyl lithium
DME=dimethoxyethane
DIBALH=diisobutylaluminum hydride
HOBT=1-hydroxybenzotriazole
CBZ=benzyloxycarbonyl
PCC=pyridinium chlorochromate
TBTU=O-(benzotriazol-1-yl)-N,N,N′,N′-tetramethyluronium tetrafluoroborate
DEBPT=3-(diethoxyphosphoryloxy)-1,2,3-benzotriazin-4(3H)-one
BOP=benzotriazole-1-yl-oxy-tris-(dimethylamino)-phosphoniumhexafluorophosphate

### Section 1: Synthesis of Benzoyl or Pyridyl Carbonyl Piperazines

WO-2000076521 (W. S. Blair, et al.) described the preparation of non-substituted and substituted benzoyl or pyridyl carbonyl piperazine in detail. The corresponding deuterated non-substituted and substituted benzoyl or pyridinyl carbonyl piperazine can be prepared in the same processes by using deuterated starting materials instead.
For example, WO-2000076521 described mono-benzoyl piperazines could be synthesized by treatmeant with 2 equivalents of n-butyllithium, followed by the addition of benzoyl chloride at room temperature (Scheme 1-1).

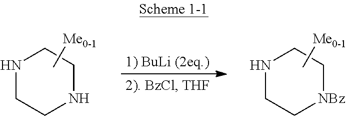

Correspondingly, in this invention, (benzoly-D5)-piperazine-2,2,3,3,5,5,6,6-D8 can be prepared from piperazine-2,2,3,3,5,5,6,6-D8 and D5-benzoyl chloride, shown in Scheme 1-2. 2 eq. of BuLi was added into the solution of 1 eq. of piperazine-2,2,3,3,5,5,6,6-D8 in THF and the resulting mixture was stirred at room temperature for 30 minutes. Then, D5-benzoyl chloride (1 eq.) was added to form (benzoly-D5)-piperazine-2,2,3,3,5,5,6,6-D8.

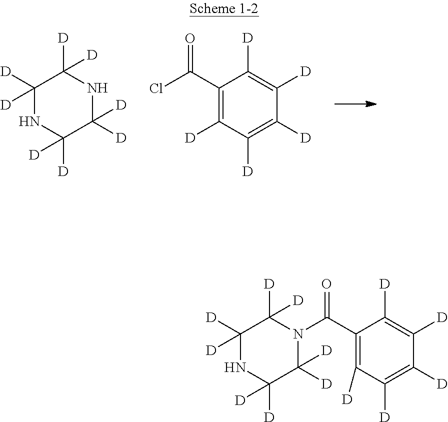

Similarly, benzol-piperazine-2,2,3,3,5,5,6,6-D8 can be prepared from piperazine-2,2,3,3,5,5,6,6-D8 and benzoyl chloride, shown in Scheme 1-3. benzol-piperazine-2,2,3,3,5,5,6,6-D8 is also commercially available.

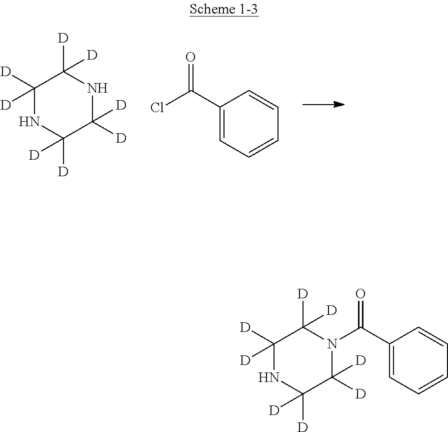

And, (benzoly-D5)-piperazine can be prepared from piperazine and D5-benzoyl chloride, shown in Scheme 1-4.

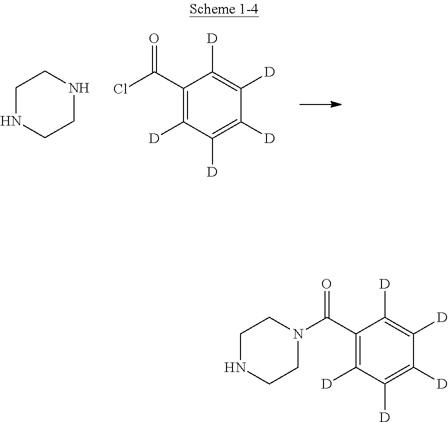 [SMILES: C1CNCCN1 ; [2H]c1c([2H])c([2H])c(C(=O)Cl)c([2H])c1[2H] ; [2H]c1c([2H])c([2H])c(C(=O)N2CCNCC2)c([2H])c1[2H]]

### Section 2: Synthesis of Phenyl or Pyridyl Tetrazolyl Piperazines

US-2007249579 (T. Wang, et al.) described the preparation of non-substituted and substituted phenyl or pyridyl tetrazolyl piperazine in detail. The corresponding deuterated non-substituted and substituted phenyl or pyridyl tetrazolyl piperazine can be prepared in the same processes by using deuterated starting materials instead.
For instance, US-2007249579 illustrated phenyl or pyridyl tetrazolyl piperazines could be prepared by reacting piperazine and phenyl or pyridyl tetrazolyl halide. As shown in Scheme 2-1, an excess of piperazine (5-10 eq.) with or without an excess of base (e.g., Et₃N, iPr₂NEt, NaH or Buli) was added to a solution of phenyl or pyridyl tetrazolyl halide in THF, dioxane or DMF. The reaction was carried out for 17 hours to 72 hours at room temperature or 115° C.

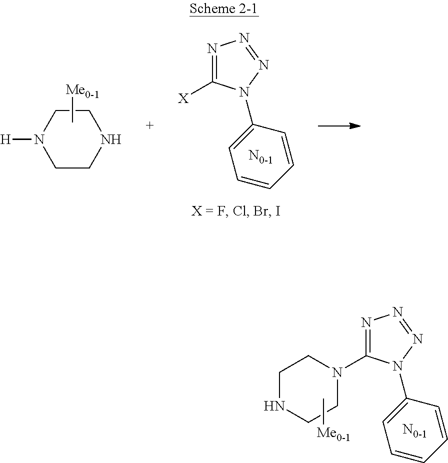

When a deuterated agent is used instead, the same process of Scheme 2-1 offers deuterated phenyl or pyridyl tetrazolyl piperazines. One specific example is shown in Scheme 2-2. An excess of iPr₂NEt was added to the solution of piperazine-2,2,3,3,5,5,6,6-D8 and 5-chloro-1-phenyl-1H-tetrazole in THF. The reaction was carried out at 115° C. for 72 hours to deliver 1-(1-phenyl-1H-tetrazol-5-yl)piperazine-2,2,3,3,5,5,6,6-D8.

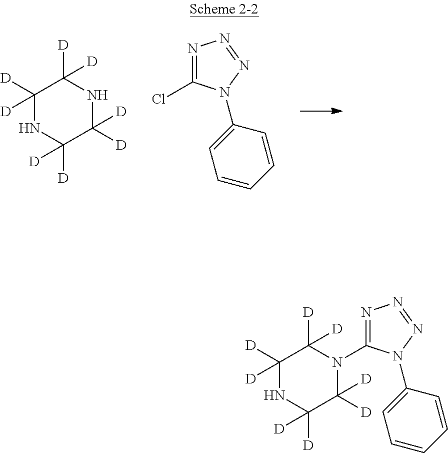

In US-2007249579, phenyl or pyridyl tetrazolyl piperazines were also prepared by reacting N-Boc-piperazine and phenyl or pyridyl tetrazolyl halide, shown in Scheme 2-3. An excess of base (1-20 eq., such as Et₃N, iPr₂Net, NaH or BuLi), was added to a solution of N-Boc-piperazine (2-5 eq.) in THF, dioxane or DMF, followed by addition of phenyl or pyridyl tetrazolyl halide (1 eq.). The reaction was carried out for 17 hours at room temperature or 115° C. to afford N-Boc phenyl or pyridyl tetrazolyl piperazine. Then, the Boc group could be removed under acidic conditions, using, for example, TFA, HCl, HOAc and H₂SO₄.

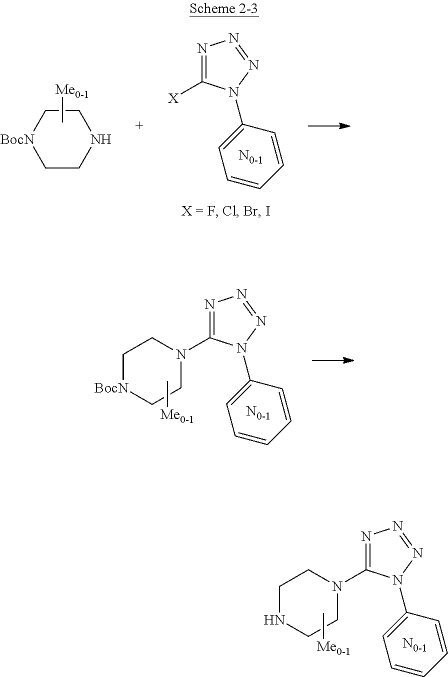

When a deuterated agent is used instead, the same process of Scheme 2-3 would offer deuterated phenyl or pyridyl tetrazolyl piperazines.
Another example in US-2007249579 was the synthesis of 1-(1-(pyridin-2-yl)-1H-tetrazol-5-yl)piperazine, shown in Scheme 2-4. 2-Aminopyridine reacted with thiocarbonyl diimidazole in methylene chloride at 25° C. gave 2-isothiocyanatopyridine, which further coupled with Boc piperazine-1-carboxylate to N-Boc-4-(pyridin-2-ylcarbamothioyl)piperazine. N-Boc-4-(pyridin-2-ylcarbamothioyl)piperazine was methylated with MeI, using potassium carbonate as base in DMSO to produce N-Boc-4-(methylthio(pyridin-2-ylimino)methyl)piperazine. Then, in DMF with mercury(II)chloride, N-Boc-4-(methylthio(pyridin-2-ylimino)methyl)piperazine reacted with an excess of sodium azide for 19 days at 25° C. to generate 1-(1-(pyridin-2-yl)-1H-tetrazol-5-yl)piperazine. Finally, Boc deprotection was performed using HCl in dioxane.

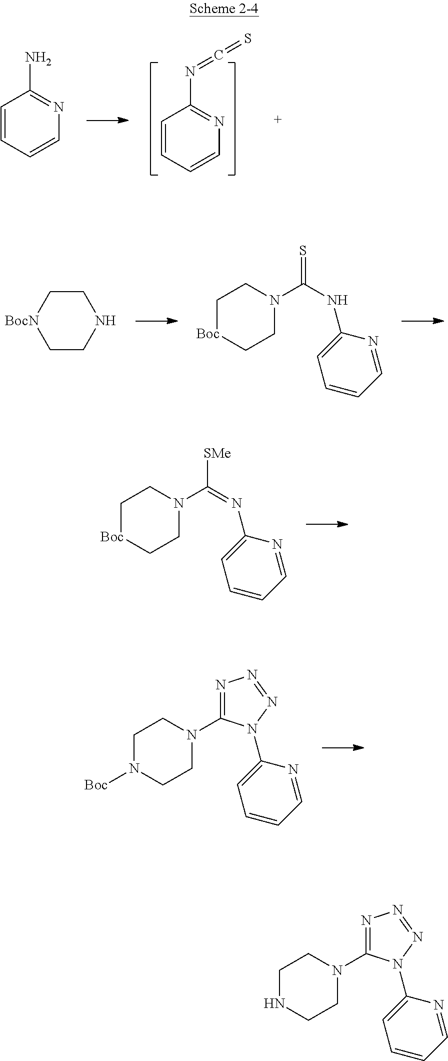

By following the same process of Scheme 2-4, commercially available N-Boc-piperazine-2,2,3,3,5,5,6,6-D8 and 2-amino pyridine could lead to 1-(1-(pyridin-2-yl)-1H-tetrazol-5-yl)piperazine-2,2,3,3,5,5,6,6-D8, shown in Scheme 2-5.

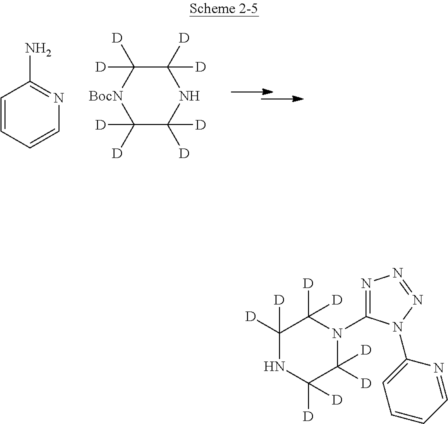

Additionally, staring with commercially available 2-amino-pyridine-3,4,5,6-D4 and N-Boc-piperazine, the same process as Scheme 2-4 would produce 1-(1-(pyridin-2-yl-3,4,5,6-D4)-1H-tetrazol-5-yl)piperazine (Scheme 2-6).

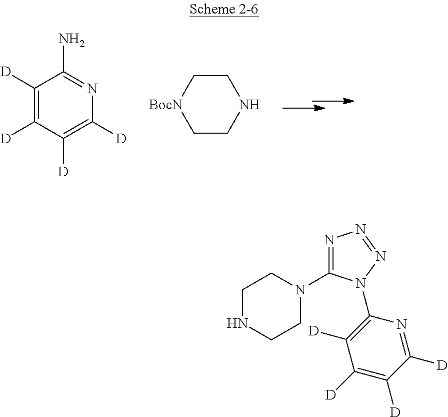 [SMILES: [2H]c1nc(N)c([2H])c([2H])c1[2H] ; CC(C)(C)OC(=O)N1CCNCC1 ; [2H]c1nc(-n2nnnc2N2CCNCC2)c([2H])c([2H])c1[2H]]

And, staring with commercially available 2-amino-pyridine-3,4,5,6-D4 and N-Boc-piperazine-2,2,3,3,5,5,6,6-D8, the same process as Scheme 2-4 would produce 1-(1-(pyridine-2-yl-3,4,5,6-D4)-1H-tetrazol-5-yl)piperazine-2,2,3,3,5,5,6,6-D8 (Scheme 2-7).

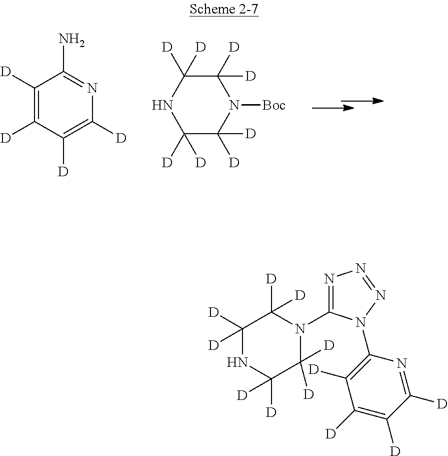

Similarly, by following the Scheme 2-4, commercially available isothiocyanatobenzene-D5 and N-Boc-piperazine could lead to 1-((1-phenyl-D5)-1H-tetrazol-5-yl)piperazine, shown in Scheme 2-8.

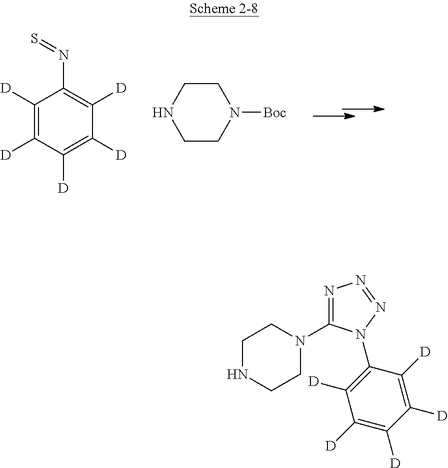

Also, when commercially available isothiocyanatobenzene-D5 and N-Boc-piperazine-2,2,3,3,5,5,6,6-D8 are used, the same process as Scheme 2-4 could lead to 1-((1-phenyl-D5)-1H-tetrazol-5-yl)piperazine-2,2,3,3,5,5,6,6-D8, shown in Scheme 2-9.

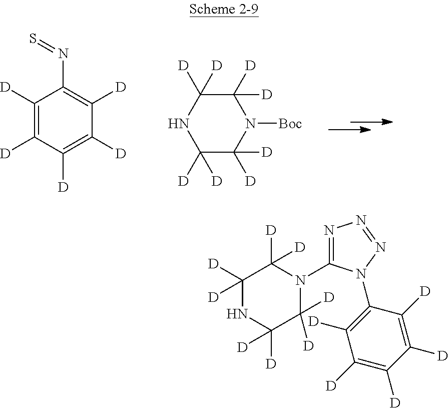 [SMILES: [2H]c1c([2H])c([2H])c(N=S)c([2H])c1[2H] ; [2H]C1([2H])NC([2H])([2H])C([2H])([2H])N(C(=O)OC(C)(C)C)C1([2H])[2H] ; [2H]c1c([2H])c([2H])c(-n2nnnc2N2C([2H])([2H])C([2H])([2H])NC([2H])([2H])C2([2H])[2H])c([2H])c1[2H]]

### Section 3: Synthesis of Cyano Phenyl or Pyridyl Alkenyl Piperidines

US-2004063744 (T. Wang, et al.) described the preparation of non-substituted and substituted cyano phenyl or pyridyl alkenyl piperidines in detail. The corresponding deuterated non-substituted and substituted cyano phenyl or pyridyl alkenyl piperidines can be prepared by the same procedures using deuterated starting materials instead.
For example, in US-2004063744, cyano phenyl or pyridyl alkenyl piperidine was made by the reaction of N-Boc-4-piperidone and phenyl or pyridyl acetonitrile in THF at room temperature for 17 to 72 hours, using NaHMDS as base (Scheme 3-1). Sequentially, Boc group was removed under acidic condition with TFA or HCl in CH₂Cl₂ or dioxane, to give cyano phenyl or pyridyl alkenyl piperidine

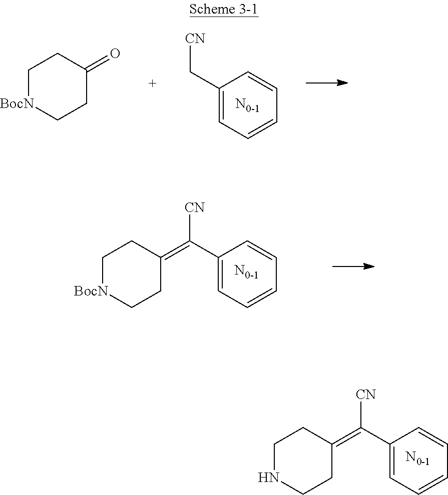 [SMILES: CC(C)(C)OC(=O)N1CCC(=O)CC1 ; N#CCc1ccccc1 ; CC(C)(C)OC(=O)N1CCC(=C(C#N)c2ccccc2)CC1 ; N#CC(=C1CCNCC1)c1ccccc1]

When a deuterated agent is used instead in this invention, the same process of Scheme 3-1 would generate deuterated cyano phenyl or pyridyl alkenyl piperidine. For instance, 4-piperidone-3,3,5,5-D4 is commercially available. N-Boc-piperidone-3,3,5,5-D4 can be prepared from 4-piperidone-3,3,5,5-D4 and Boc₂O in THF, CH₂Cl₂ or dioxane using Et₃N or iPr₂NEt as base. By following the same process of Scheme 3-1, N-Boc-piperidone-3,3,5,5-D4 could lead to N-Boc cyano phenyl or pyridyl alkenyl piperidone-3,3,5,5-D4 shown in Scheme 3-2.

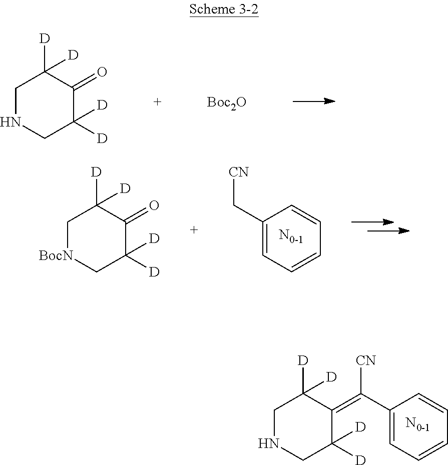

### Section 4: Intermediate ACOCOOH:

The preparation of template A-CO—CO—OH has been described in detail in WO-2001062255 (T. Wang, et al.) and WO-2002062423 (T. Wang, et al.).
Particularly, 2-(4-methoxy-7-(3-substituted/unsubstituted-1H-1,2,4-triazol-1-yl)-1H-pyrrolo[2,3-c]pyridin-3-yl)-2-oxoacetic acid was prepared from 7-bromo or 7-chloro-4-Br-1H-pyrrolo[2,3-c]pyridine and 3-substituted-1H-1,2,4-triazole or parent 1,2,4-triazole. As shown in Scheme 4-1, 4-bromo-7-chloro-6-azaindole coupled with NaOMe with Cu or Cu (I) salt (e.g., CuBr, CuI) to offer 4-methoxy-7-chloro-6-azaindole. 4-Methoxy-7-chloro-6-azaindole then reacted with 1,2,4-triazole or 3-substituted-1,2,4-triazole, in the presence of Cu or Cu (I) salt without base or with base (e.g., K₂CO₃, Cs₂CO₃) to give 4-methoxy-7-(3-H or substituted-1,2,4-triazol-1-yl)-6-azaindole. Acylation of 4-methoxy-7-(3-H or substituted-1,2,4-triazol-1-yl)-6-azaindole with methyl or ethyl 2-chloro-2-oxoacetate in the presence of an excess of AlCl₃, followed by hydrolysis, generated 2-(4-methoxy-7-(3-H or substituted-1H-1,2,4-triazol-1-yl)-1H-pyrrolo[2,3-c]pyridin-3-yl)-2-oxoacetic acid.

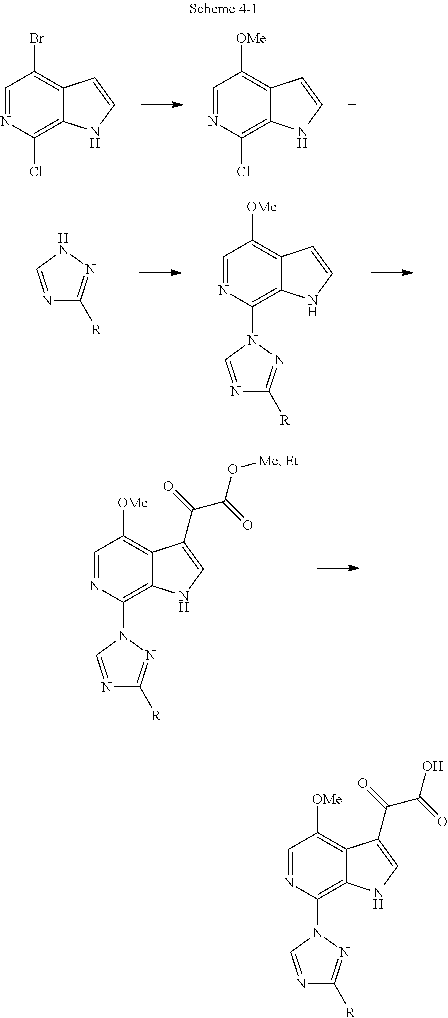

When a deuterated agent is used instead in this invention, the same process of Scheme 4-1 would offer deuterated 2-(4-methoxy-7-(3-H or substituted-1H-1,2,4-triazol-1-yl)-1H-pyrrolo[2,3-c]pyridin-3-yl)-2-oxoacetic acid. For instance, 1,2,4-triazole-D2 and 1,2,4-triazole-D3 are both commercially available. By following the same process of Scheme 4-1, 1,2,4-triazole-D2 and 1,2,4-triazole-D3 would lead to 2-(4-methoxy-7-(1,2,4-triazol-1-yl-D2)-1H-pyrrolo[2,3-c]pyridine-3-yl)-2-oxoacetic acid, as shown in Scheme 4-2.

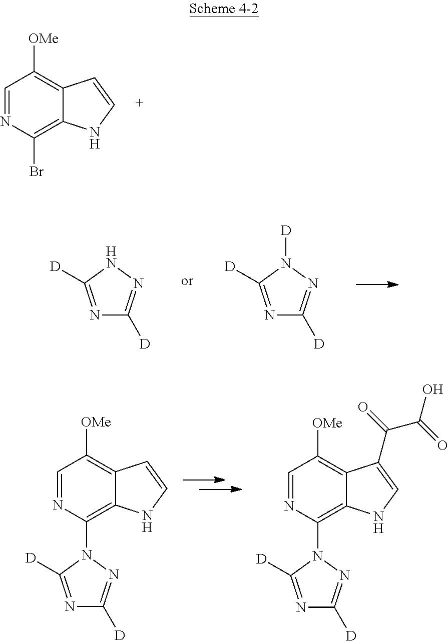

Similarly, 1,2,4-triazole-1D can be prepared according to the documented procedure (Maquestiau, A.; Van Haverbeke, Y.; Flammang, R. Fragmentation of 1,2,4-triazole under electron impact. Organic Mass Spectrometry (1972), 6(10), 1139-44). By following the same process of Scheme 4-1, 1,2,4-triazole-D1 could lead to 2-(4-methoxy-7-(1,2,4-triazol-1-yl-D1)-1H-pyrrolo[2,3-c]pyridine-3-yl)-2-oxoacetic acid, as shown in Scheme 4-3.

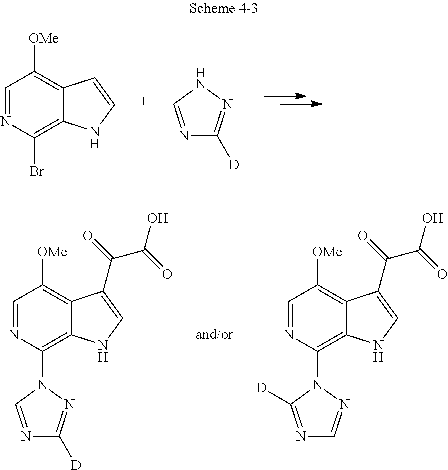 [SMILES: COc1cnc(Br)c2[nH]ccc12 ; [2H]c1nc[nH]n1 ; [2H]c1ncn(-c2ncc(OC)c3c(C(=O)C(=O)O)c[nH]c23)n1 ; [2H]c1ncnn1-c1ncc(OC)c2c(C(=O)C(=O)O)c[nH]c12]

And, as shown in Scheme 4-4, 3-(methyl-D3)-1,2,4-triazole could lead to 2-(4-methoxy-7-(3-(methyl-D3)-1H-1,2,4-triazol-1-yl)-1H-pyrrolo[2,3-c]pyridine-3-yl)-2-oxoacetic acid (equation 1, Scheme 4-4). And, 3-(methyl-D3)-1,2,4-triazole-1D could lead to 2-(4-methoxy-7-(3-(methyl-D3)-1H-1,2,4-triazol-1-yl-1D)-1H-pyrrolo[2,3-c]pyridine-3-yl)-2-oxoacetic acid (equation 2, Scheme 4-4), and, 3-methyl-1,2,4-triazole-1D could lead to 2-(4-methoxy-7-(3-methyl-1H-1,2,4-triazol-1-yl-1D)-1H-pyrrolo[2,3-c]pyridine-3-yl)-2-oxoacetic acid (equation 3, Scheme 4-4).

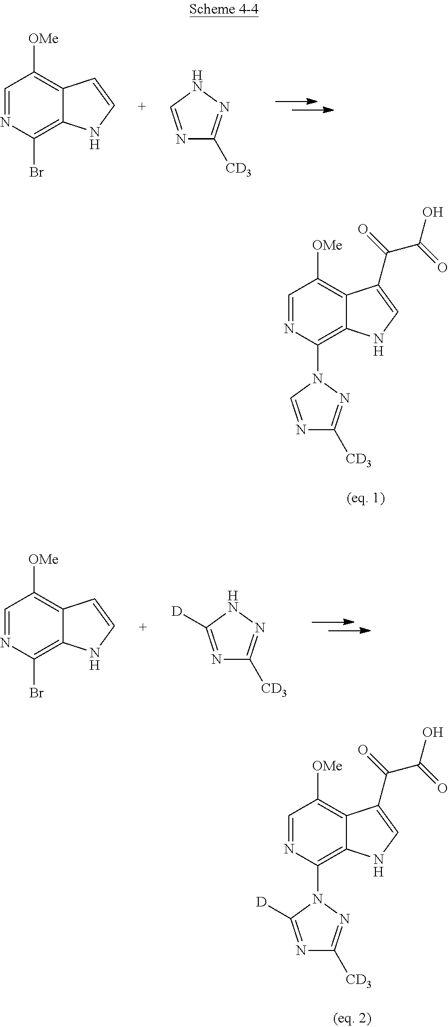 [SMILES: COc1cnc(Br)c2[nH]ccc12 ; [2H]C([2H])([2H])c1nc[nH]n1 ; [2H]C([2H])([2H])c1ncn(-c2ncc(OC)c3c(C(=O)C(=O)O)c[nH]c23)n1 ; COc1cnc(Br)c2[nH]ccc12 ; [2H]c1nc(C([2H])([2H])[2H])n[nH]1 ; [2H]c1nc(C([2H])([2H])[2H])nn1-c1ncc(OC)c2c(C(=O)C(=O)O)c[nH]c12]

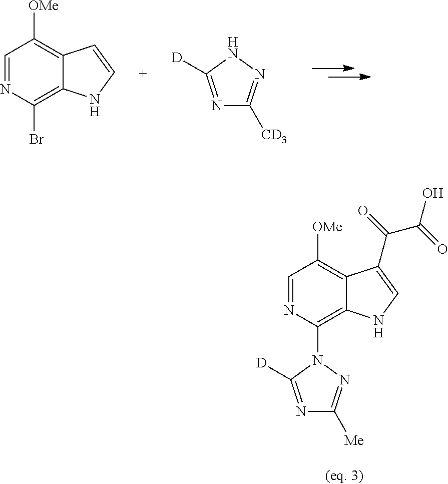 [SMILES: COc1cnc(Br)c2[nH]ccc12 ; [2H]c1nc(C([2H])([2H])[2H])n[nH]1 ; [2H]c1nc(C)nn1-c1ncc(OC)c2c(C(=O)C(=O)O)c[nH]c12]

As shown in Scheme 4-5, Jones and Ainsworth (J. Am. Chem. Soc. 1955, 77, 1538) reported a synthesis of 3-methyl-1,2,4-triazole from acetyl chloride and thiosemicarbazide. Acetyl chloride and thiosemicarbazide reacted in pyridine afforded 1-acetylthiosemicarbazide, which was treated in methanol by sodium methylate to cyclize to 3-methyl-1,2,4-triazole-5-thiol. The mercapto group of 3-methyl-1,2,4-triazole-5-thiol was removed by nitric acid oxidation to give 3-methyl-1,2,4-triazole.

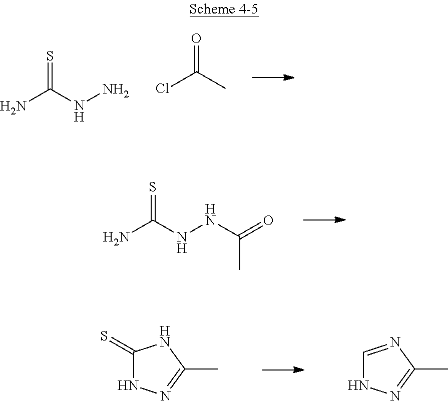

When commercially available acetyl chloride-D3 is used, the same process of Scheme 4-5 would produce 3-(methyl-D3)-1,2,4-triazole, shown in Scheme 4-6.

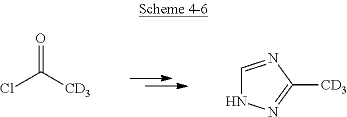 [SMILES: [2H]C([2H])([2H])C(=O)Cl ; [2H]C([2H])([2H])c1nc[nH]n1]

Another synthsis of 3-methyl 1,2,4-triazole was reported by Katritzky, Lue and Yannakopoulou (Tetrahedron 1990, 46, 641, Scheme 4-7). 1,2,4-Triazole, pyrrolidine and formaldehyde reacted to generate 1-(1-pyrrolidinomethyl)-1,2,4-triazole. Deprotonation of 1-(1-pyrrolidinomethyl)-1,2,4-triazole, followed by addition of methyl iodide led to 5-methyl-1-(1-pyrrolidinomethyl)-1,2,4-triazole. Finally, NaBH₄ in ethanol removed the pyrrolidinomethyl group to afford 3-methyl-1,2,4-triazole.

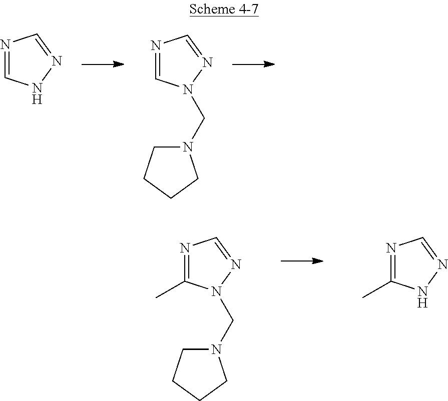

By following the same procedure of Scheme 4-7, 1,2,4-triazole and methyl iodide-D3 could lead to 3-(methyl-D3)-1,2,4-triazole (equation 1, Scheme 4-8). And, 1,2,4-triazole-3,5-2D and methyl iodide-D3 could lead to 3-(methyl-D3)-1,2,4-triazole-5-D (equation 2, Scheme 4-8), and, 1,2,4-triazole-3, 5-2D and methyl iodide could lead to 3-methyl-1,2,4-triazole-5-D (equation 3, Scheme 4-8).

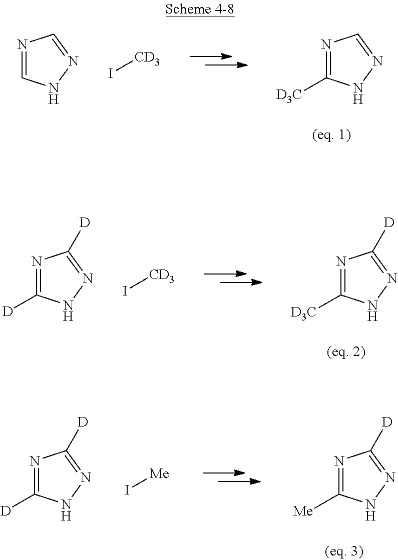

Furthermore, commercially available CD₃OH would react with 4-bromo-7-chloro-6-azaindole under the same conditions described in Scheme 4-1 to afford 4-(methoxy-D3)-7-chloro-6-azaindole (Scheme 4-9). Following coupling with 3-methyl-1,2,4-triazole, (methyl-D3)-1,2,4-triazole and (methyl-D3)-1,2,4-triazole-1D would lead to 2-(4-(methoxy-D3)-7-(3-methyl-1H-1,2,4-triazol-1-yl)-1H-pyrrolo[2,3-c]pyridine-3-yl)-2-oxoacetic acid (equation 1, Scheme 4-10), 2-(4-(methoxy-D3)-7-(3-(methyl-D3)-1H-1,2,4-triazol-1-yl)-1H-pyrrolo[2,3-c]pyridine-3-yl)-2-oxoacetic acid (equation 2, Scheme 4-10) and 2-(4-(methoxy-D3)-7-(3-(methyl-D3)-1H-1,2,4-triazol-1-yl-1D)-1H-pyrrolo[2,3-c]pyridine-3-yl)-2-oxoacetic acid (equation 3, Scheme 4-10).

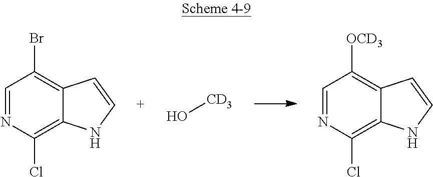

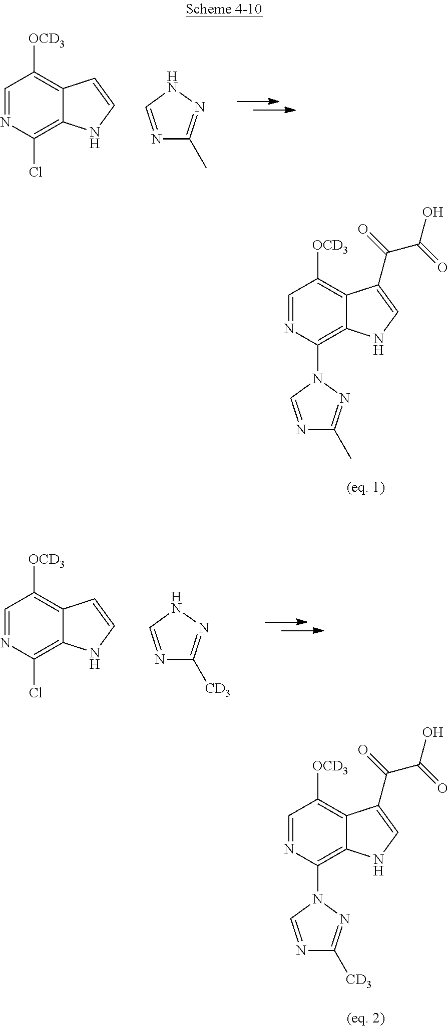

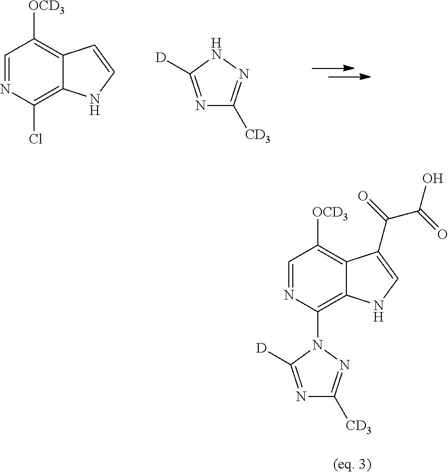

### Commercially Available Deuterium Containing Agents:

The following agents are commercially available, which would be used as is during the synthesis of intermediates described in sections 1, 2, 3 and 4:

| Agent | Commercial Source |
|---|---|
| 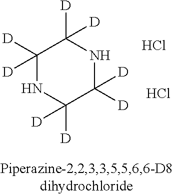 [SMILES: [2H]C1([2H])NC([2H])([2H])C([2H])([2H])NC1([2H])[2H] ; Cl ; Cl] | Sigma-Aldrich: 448125-1g C/D/N Isotopes, Inc.: D-1812 Kanto Chemical Co., Inc.: 49132-63 |
| 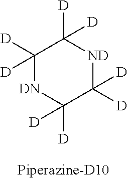 | C/D/N Isotopes, Inc.: D-5487 Kanto Chemical Co., Inc.: 49132-62 |
| 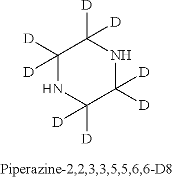 [SMILES: [2H]C1([2H])NC([2H])([2H])C([2H])([2H])NC1([2H])[2H]] | C/D/N Isotopes, Inc.: D-6283 Combiphos Catalysts, Inc.: 083D |
| 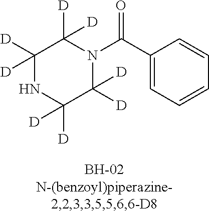 [SMILES: [2H]C1([2H])NC([2H])([2H])C([2H])([2H])N(C(=O)c2ccccc2)C1([2H])[2H]] | C/D/N Isotopes, Inc.: D-6285 |
| 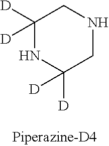 | Ramidus: 3552 |
| 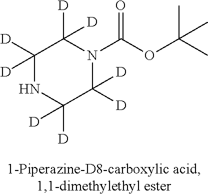 [SMILES: [2H]C1([2H])NC([2H])([2H])C([2H])([2H])N(C(=O)OC(C)(C)C)C1([2H])[2H]] | C/D/N Isotopes, Inc.: D-6714 Toronto Research Chemicals: B662002 |
| 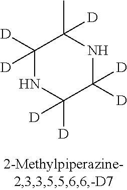 | C/D/N Isotopes, Inc.: D-6609 |
| 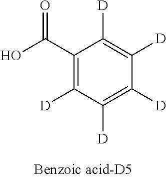 [SMILES: [2H]c1c([2H])c([2H])c(C(=O)O)c([2H])c1[2H]] | Cambridge Isotope Laboratories, Inc.: DLM-122 ChemService Inc: FD702-1 Sigma-Aldrich: 217158-1g Acros Organics: 20277-0010 C/D/N Isotopes, Inc.: D-168 Carbocore, Inc: DU-0010 Kanto Chemical Co., Inc.: 20277-1A MP Biomedicals, Inc.: MD 102 |
| 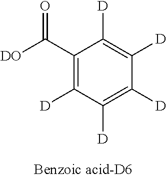 | Sigma-Aldrich: 616796 C/D/N Isotopes, Inc.: D-1156 Carbocore, Inc: DU-0010 Kanto Chemical Co., Inc.: 49128-74 |
| 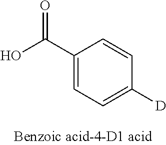 | C/D/N Isotopes, Inc.: D-6401 |
| 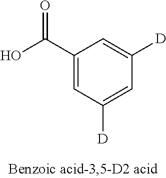 [SMILES: [2H]c1cc([2H])cc(C(=O)O)c1] | C/D/N Isotopes, Inc.: D-6472 |
| 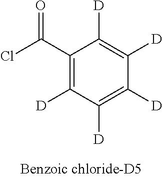 [SMILES: [2H]c1c([2H])c([2H])c(C(=O)Cl)c([2H])c1[2H]] | Sigma-Aldrich: 366048-1g Acros Organics: 27884-0010 C/D/N Isotopes, Inc.: D-315 Kanto Chemical Co., Inc.: 27884-1A |
| 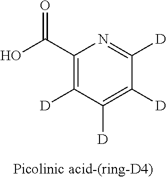 [SMILES: [2H]c1nc(C(=O)O)c([2H])c([2H])c1[2H]] | Sigma-Aldrich: 615757 C/D/N Isotopes, Inc.: D-5289 Kanto Chemical Co., Inc.: 49132-61 |
| 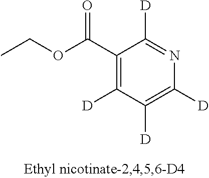 | Cambridge Isotope Laboratories, Inc.: DLM-2872 C/D/N Isotopes, Inc.: D-4173 Kanto Chemical Co., Inc.: 49127-40 |
| 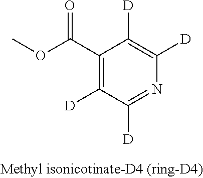 | Sigma-Aldrich: 615412 |
| 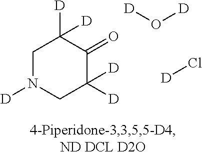 | C/D/N Isotopes, Inc.: D-6394 |
| 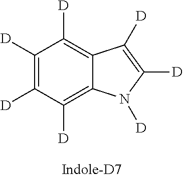 | Sigma-Aldrich: 674621 C/D/N Isotopes, Inc.: D-1897 |
| 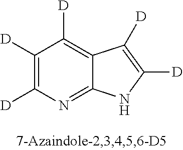 [SMILES: [2H]c1nc2[nH]c([2H])c([2H])c2c([2H])c1[2H]] | Wako Pure Chemical Industries, Inc: 014-22501 |
| 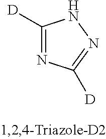 | Combiphos Catalysts, Inc.: 2020D |
| 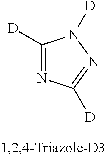 | C/D/N Isotopes, Inc.: D-5453 Kanto Chemical Co., Inc.: 49134-96 |
| 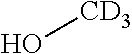 | Cambridge Isotope Laboratories, Inc.: DLM-598 Sigma-Aldrich: 343854-10g Acros Organics: 30073-0100 C/D/N Isotopes, Inc.: D-67 Kanto Chemical Co., Inc.: 30073-1A |
| 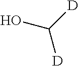 | Cambridge Isotope Laboratories, Inc.: DLM-1209 Sigma-Aldrich: 486884-5g C/D/N Isotopes, Inc.: D-758 Kanto Chemical Co., Inc.: 49129-72 |
| 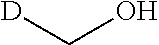 | Sigma-Aldrich: 490296 C/D/N Isotopes, Inc.: D-468 Kanto Chemical Co., Inc.: 49129-71 |
| 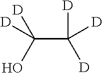 | Cambridge Isotope Laboratories, Inc.: DLM-413 Sigma-Aldrich: 489336-5g C/D/N Isotopes, Inc.: D-108 Kanto Chemical Co., Inc.: 49127-00 |
| 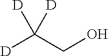 | Cambridge Isotope Laboratories, Inc.: DLM-412 Sigma-Aldrich: 329347-5g C/D/N Isotopes, Inc.: D-209 Kanto Chemical Co., Inc.: 49126-95 |
| 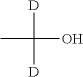 | Cambridge Isotope Laboratories, Inc.: DLM-345 Sigma-Aldrich: 347434-5g C/D/N Isotopes, Inc.: D-60 Kanto Chemical Co., Inc.: 49126-94 |
| 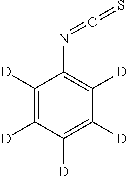 [SMILES: [2H]c1c([2H])c([2H])c(N=C=S)c([2H])c1[2H]] | C/D/N Isotopes, Inc.: D-5529 Kanto Chemical Co., Inc.: 49132-51 |
| 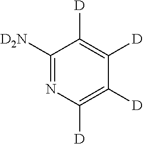 | C/D/N Isotopes, Inc.: D-3508 Kanto Chemical Co., Inc.: 49120-81 |
| 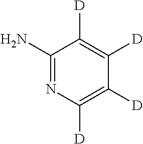 [SMILES: [2H]c1nc(N)c([2H])c([2H])c1[2H]] | Combiphos Catalysts, Inc.: 1117D |
| 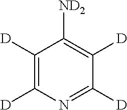 | C/D/N Isotopes, Inc.: D-5114 Kanto Chemical Co., Inc.: 49120-82 |
| 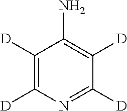 [SMILES: [2H]c1nc([2H])c([2H])c(N)c1[2H]] | Combiphos Catalysts, Inc.: 1123D Wako Pure Chemical Industries, Inc: 010-22461 |
| 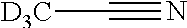 | ALFA AESAR, AVOCADO, LANCASTER: 42264-18 Sigma-Aldrich: 366544-10g TCI America: A2018 C/D/N Isotopes, Inc.: D-129 Kanto Chemical Co., Inc.: 21453-1A |
| 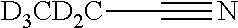 | Sigma-Aldrich: 486167-5g C/D/N Isotopes, Inc.: D-531 Kanto Chemical Co., Inc.: 49133-20 |
| 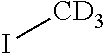 | Cambridge Isotope Laboratories, Inc.: DLM-362 Sigma-Aldrich: 176036-25g Acros Organics: 17495-0250 Kanto Chemical Co., Inc.: 49129-26 |
| 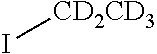 | Cambridge Isotope Laboratories, Inc.: DLM-272 Sigma-Aldrich: 324582-5g C/D/N Isotopes, Inc.: D-211 Kanto Chemical Co., Inc.: 27881-1A |
| 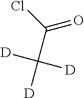 | Cambridge Isotope Laboratories, Inc.: DLM-247 Sigma-Aldrich: 175668-5g C/D/N Isotopes, Inc.: D-260 Kanto Chemical Co., Inc.: 17468-1A |
| 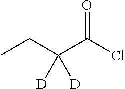 | C/D/N Isotopes, Inc.: D-350 Kanto Chemical Co., Inc.: 49123-27 |
| 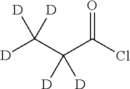 | C/D/N Isotopes, Inc.: D-5212 Kanto Chemical Co., Inc.: 49133-22 |
| 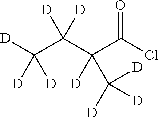 [SMILES: [2H]C([2H])([2H])C([2H])([2H])C([2H])(C(=O)Cl)C([2H])([2H])[2H]] | C/D/N Isotopes, Inc.: D-5463 Kanto Chemical Co., Inc.: 49130-13 |
| 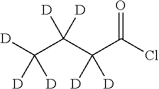 [SMILES: [2H]C([2H])([2H])C([2H])([2H])C([2H])([2H])C(=O)Cl] | C/D/N Isotopes, Inc.: D-5779 Kanto Chemical Co., Inc.: 49123-28 |
| 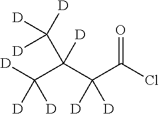 [SMILES: [2H]C([2H])([2H])C([2H])(C([2H])([2H])[2H])C([2H])([2H])C(=O)Cl] | C/D/N Isotopes, Inc.: D-6608 |
| 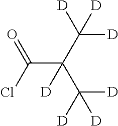 [SMILES: [2H]C([2H])([2H])C([2H])(C(=O)Cl)C([2H])([2H])[2H]] | C/D/N Isotopes, Inc.: D-6853 |
| 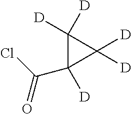 [SMILES: [2H]C1([2H])C([2H])([2H])C1([2H])C(=O)Cl] | C/D/N Isotopes, Inc.: D-7023 |

### Documented Deuterium Containing Agents:

The following agents are recorded in literature, which could be prepared accordingly.

| Structure | Literature |
|---|---|
| 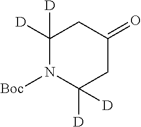 [SMILES: [2H]C1([2H])CC(=O)CC([2H])([2H])N1C(=O)OC(C)(C)C] | D. Hesk, et al. Synthesis of 3H, 14C and 2H4 labelled SCH 211803. Journal of Labelled Compounds and Radiopharmaceuticals (2007), 50(2), 131-137. |
| 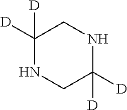 | Zeng, Dexing; Li, Shuwei. Improved CILAT reagents for quantitative proteomics. Bioorganic & Medicinal Chemistry Letters (2009), 19(7), 2059- 2061. |
| 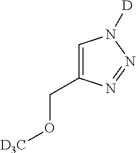 | Banert, Klaus; Hagedorn, Manfred. Reactions of unsaturated azides. 9. First isolation of allenyl azides. Angewandte Chemie (1989), 101(12), 1710-11. |
| 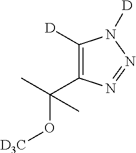 | Banert, Klaus. Reactions of unsaturated azides. 6. Synthesis of 1,2,3-triazoles from propargyl azides by rearrangement of the azido group. Indication of short-lived allenyl azides and triazafulvenes. Chemische Berichte (1989), 122(5), 911-18. |
| 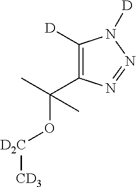 | Banert, Klaus. Reactions of unsaturated azides. 6. Synthesis of 1,2,3-triazoles from propargyl azides by rearrangement of the azido group. Indication of short-lived allenyl azides and triazafulvenes. Chemische Berichte (1989), 122(5), 911-18. |
| 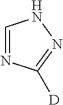 | Maquestiau, A.; Van Haverbeke, Y.; Flammang, R. Fragmentation of 1,2,4- triazole under electron impact. Organic Mass Spectrometry (1972), 6(10), 1139-44. |
| 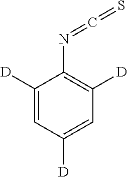 [SMILES: [2H]c1cc([2H])c(N=C=S)c([2H])c1] | Norris, Brent C.; Bielawski, Christopher W. Structurally Dynamic Materials Based on Bis(N-heterocyclic carbene)s and Bis(isothiocyanate)s: Toward Reversible, Conjugated Polymers. Macromolecules (Washington, DC, United States) (2010), 43(8), 3591-3593. |
| 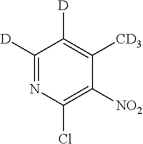 [SMILES: [2H]c1nc(Cl)c([N+](=O)[O-])c(C([2H])([2H])[2H])c1[2H]] | Gant, Thomas G.; Sarshar, Sepehr. Preparation of deuterium-incorporated cyclopropyldipyridodiazepinone derivatives for use as non-nucleoside reverse transcriptase inhibitors. WO 2008103899 |
| 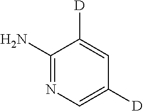 | Czarnik, Anthony. Deuterium-enriched eszopiclone. WO 2008157564. |
| 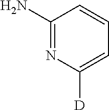 | De Bie, Dick A.; Geurtsen, Bart; Van der Plas, Henk C. On the amination of halonitropyridines. Journal of Organic Chemistry (1985), 50(4), 484-7. Czarnik, Anthony. Deuterium-enriched eszopiclone. WO 2008157564. |
| 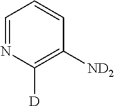 | Smolyar, N. N.; Yutilov, Yu. M. Reduction of 2-amino-3- and -5- nitropyridine derivatives with hydrazine hydrate. Russian Journal of Organic Chemistry (2009), 45(1), 115-118. |
| 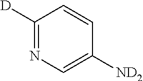 | Smolyar, N. N.; Yutilov, Yu. M. Reduction of 2-amino-3- and -5- nitropyridine derivatives with hydrazine hydrate. Russian Journal of Organic Chemistry (2009), 45(1), 115-118. |
| 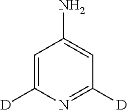 | Esaki, Hiroyoshi; Ito, Nobuhiro; Sakai, Shino; Maegawa, Tomohiro; Monguchi, Yasunari; Sajiki, Hironao. General method of obtaining deuterium-labeled heterocyclic compounds using neutral D₂O with heterogeneous Pd/C. Tetrahedron (2006), 62(47), 10954-10961. Tupitsyn, I. F.; Zatsepina, N. N.; Kolodina, N.S. Electronic interactions and infrared intensities of aromatic heterocyclic molecules. II. CH-stretching vibrations of the aromatic CH-bonds in six-membered nitrogen heterocycles and their N-oxides. Reaktsionnaya Sposobnost Organicheskikh Soedinenii (1969), 6(1), 11-23. |

### Section 5: Syntheses of the Compounds of Formula I

Detailed procedures of coupling ACOCOOH and piperazine or piperidine derivative were described in application (T. Wang, et al. WO-2001062255, T. Wang, et al. WO-2002062423, T. Wang, et al. US-2007249579 and T. Wang, et al. US-2004063744). ACOCOOH (1 eq.), piperazine or piperidine derivative(1-5 eq.), 3-(diethoxyphosphoryloxy)-1,2,3-benzotriazin-4(3H)-one (DEPBT) or O-(1H-benzotriazol-1-yl)-N,N,N′,N′-tetramethyluronium tetrafluoroborate (TBTU) (1-5 eq.) or (2-(7-Aza-1H-benzotriazole-1-yl)-1,1,3,3-tetramethyluronium hexafluorophosphate) (HATU) (1-5 eq.) and Hunig's Base or N-methyl morpholine or triethyl amine (1-100 eq.) were combined in THF or DMF. The reactions were carried out at either room temperature or increased temperature.

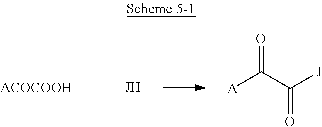

By using the deuterated intermediates described in sections 1, 2, 3 and 4, the following deuterated compounds were synthesized by using general coupling procedure (Structures 5-1).

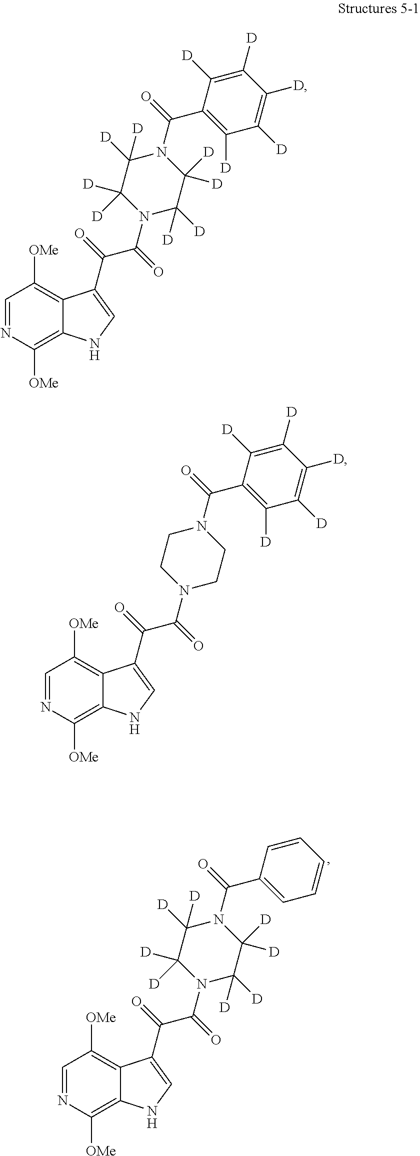

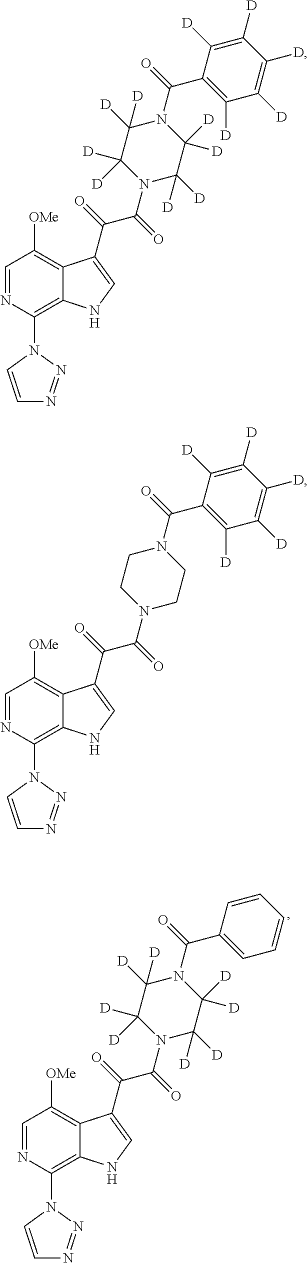

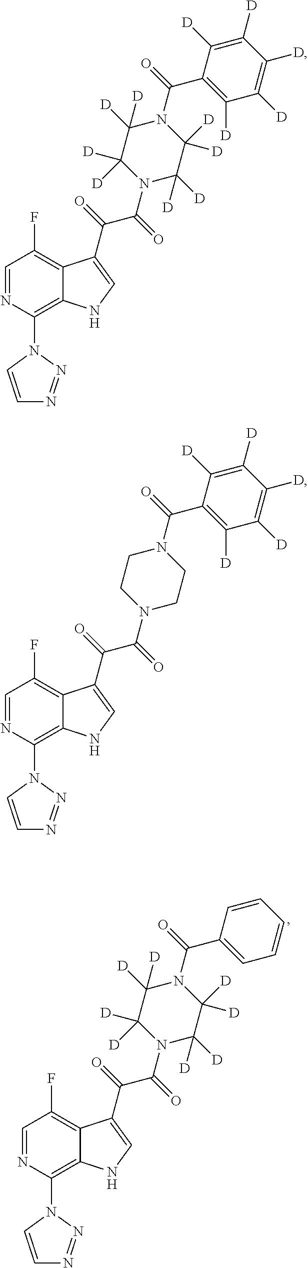

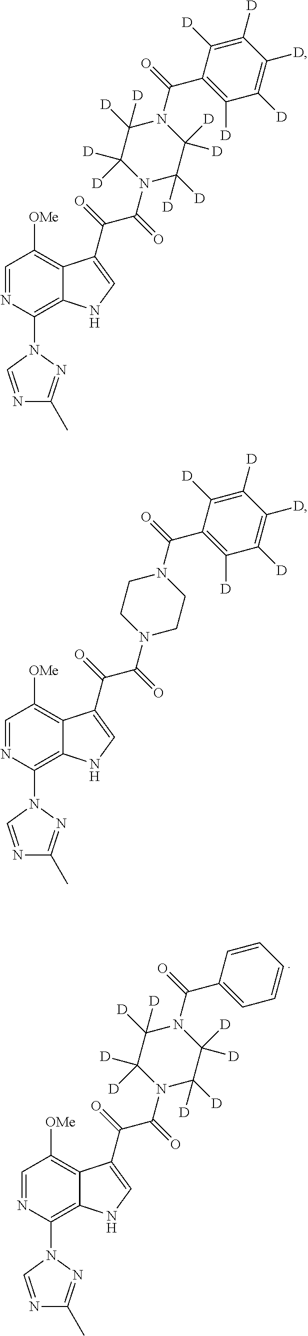

Additional examples could be prepared via the same process (Structures 5-2).

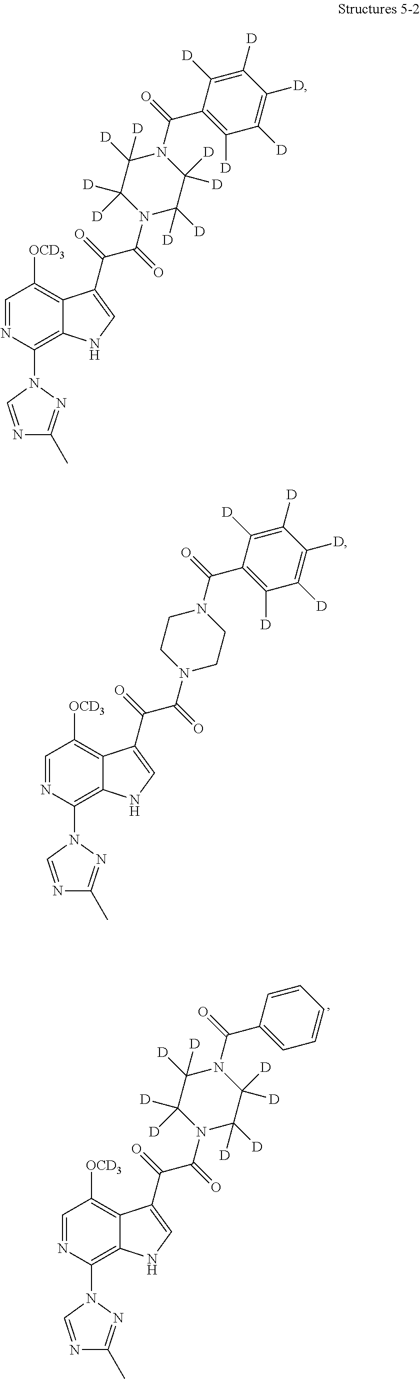

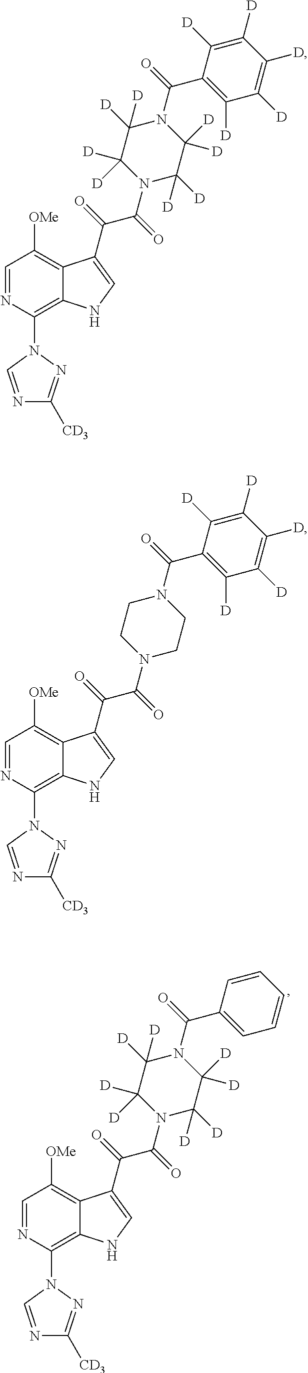

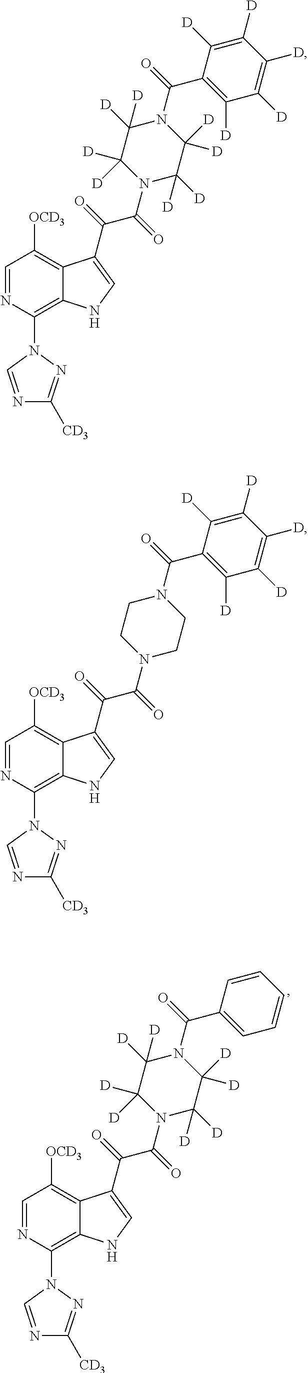

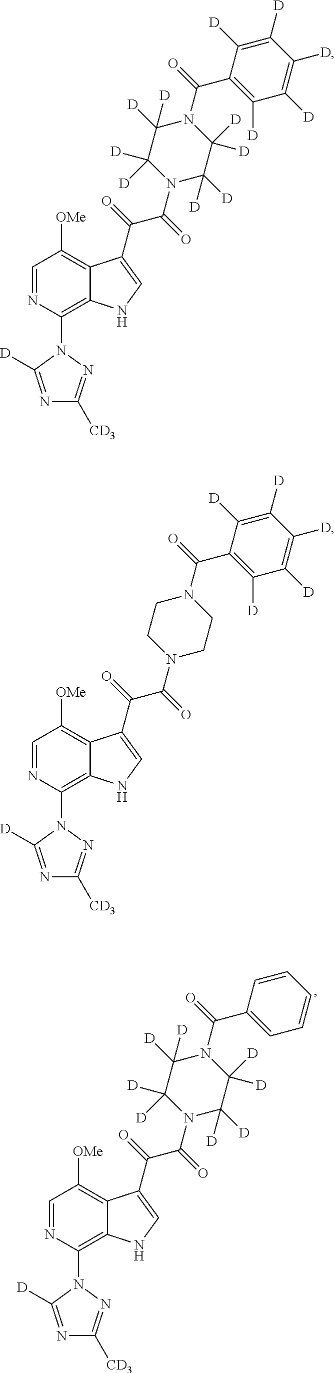

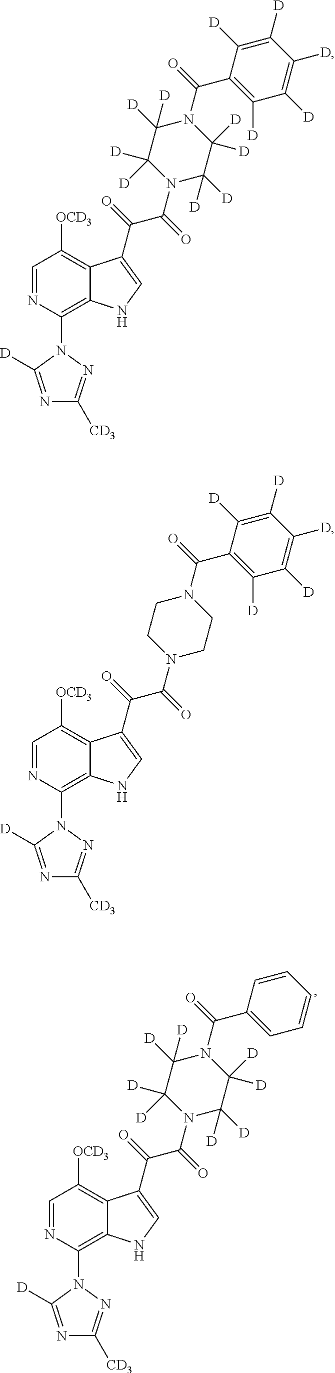

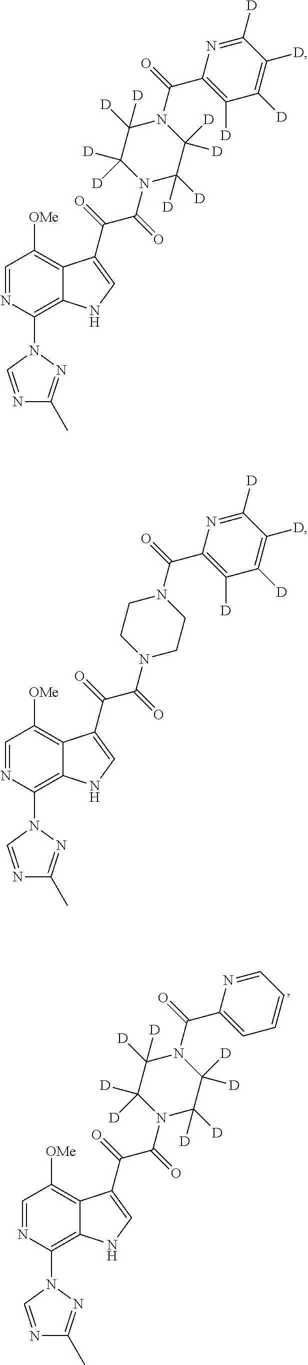

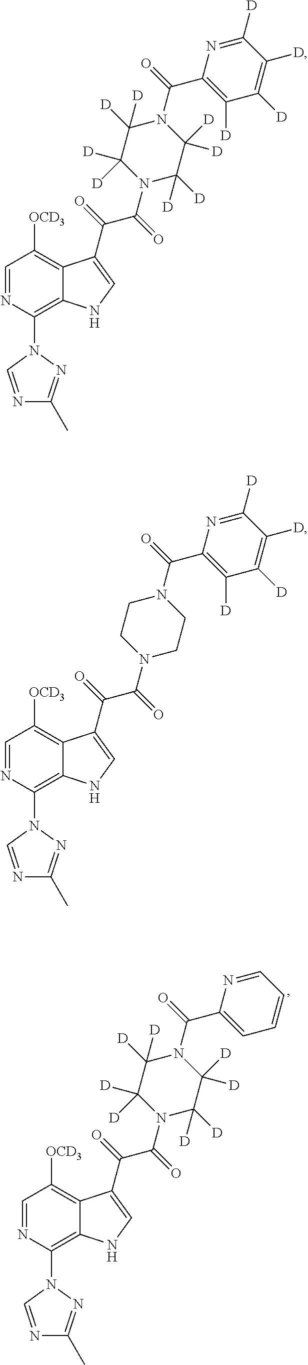

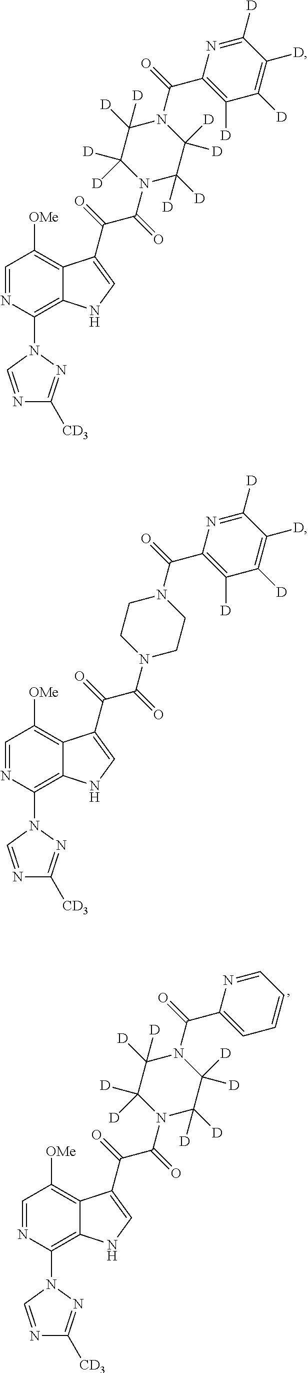

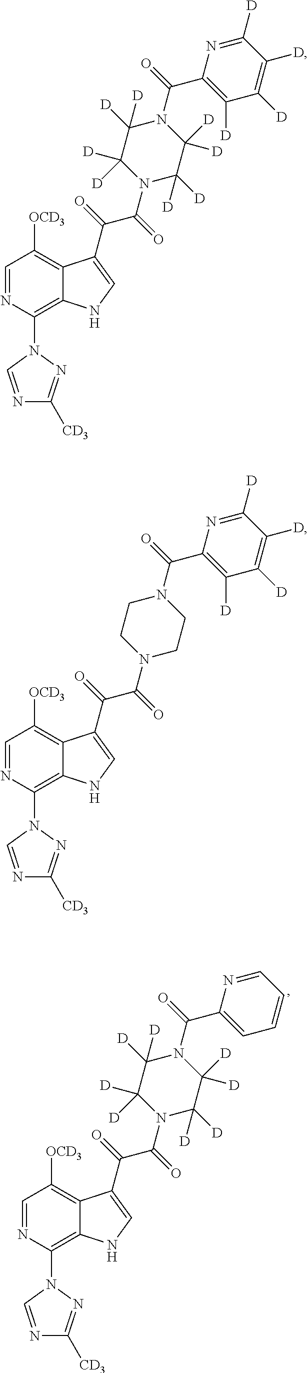

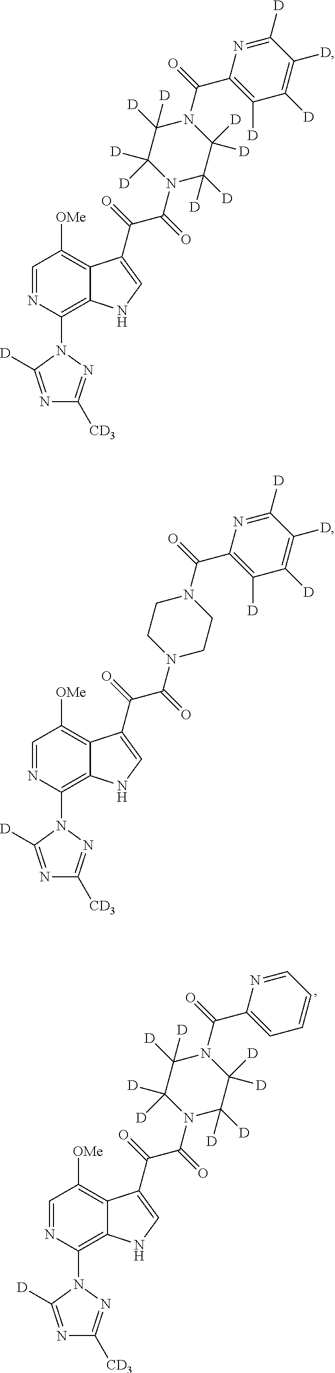

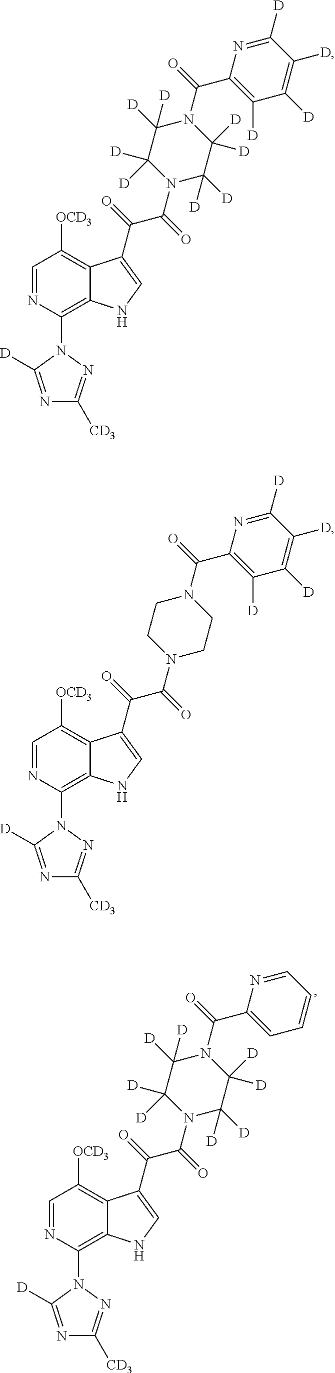

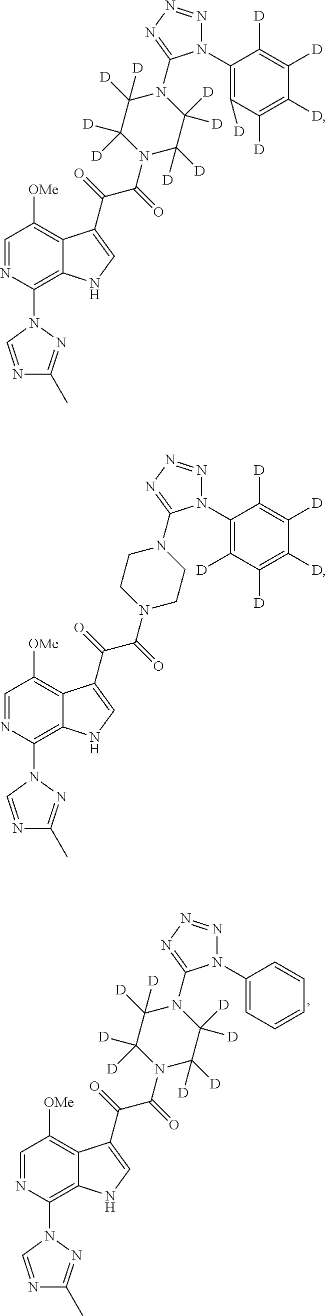

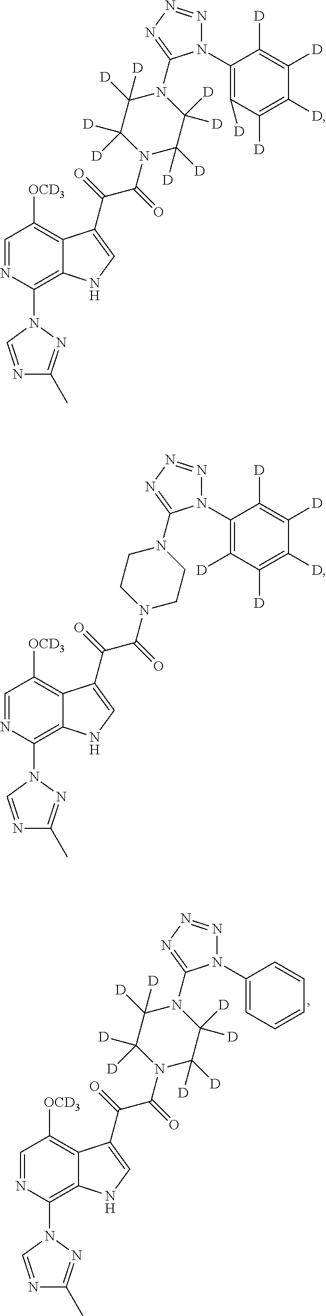

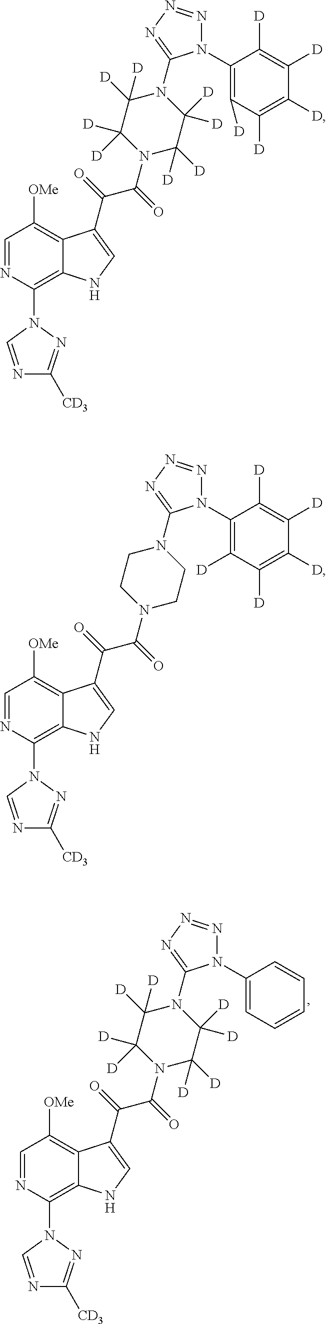

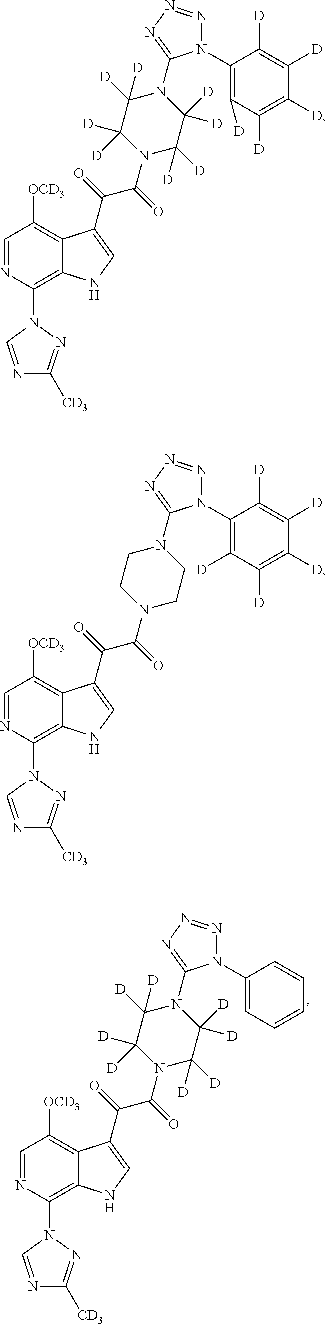

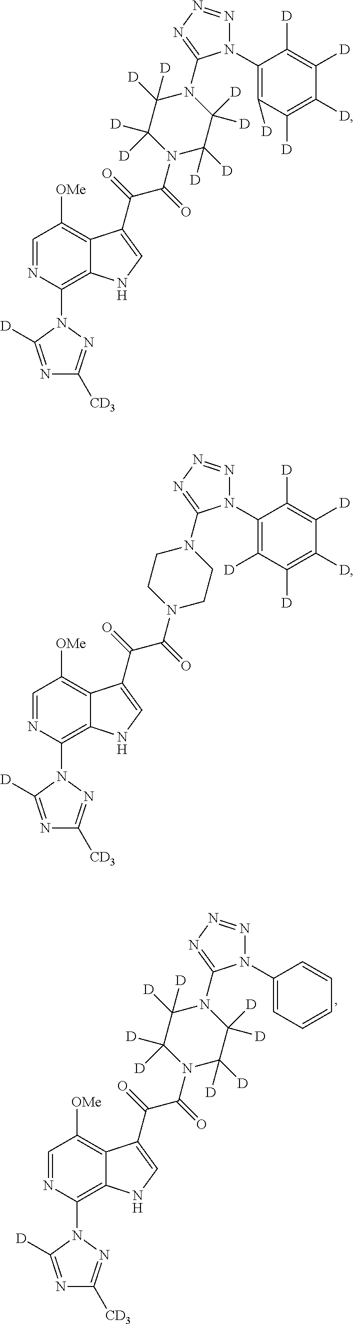

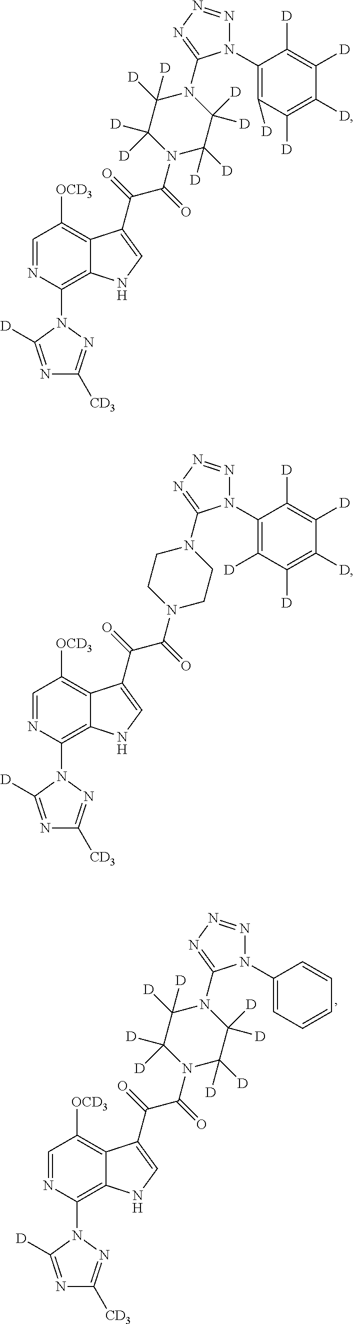

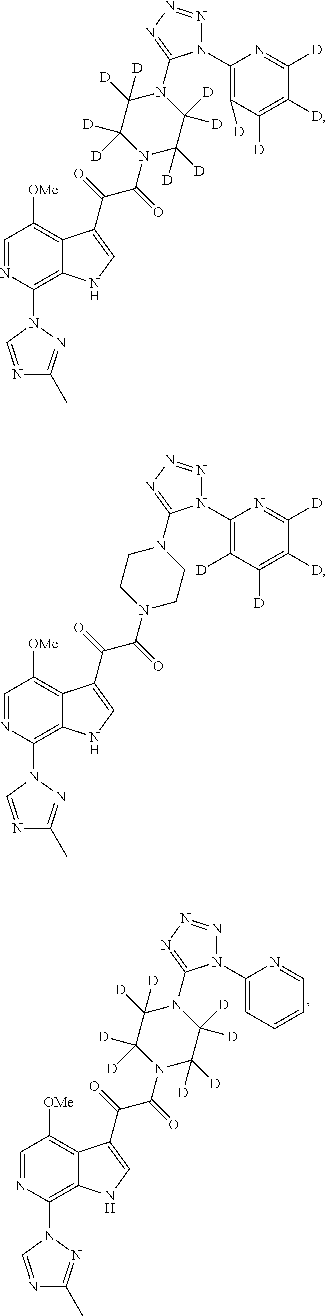

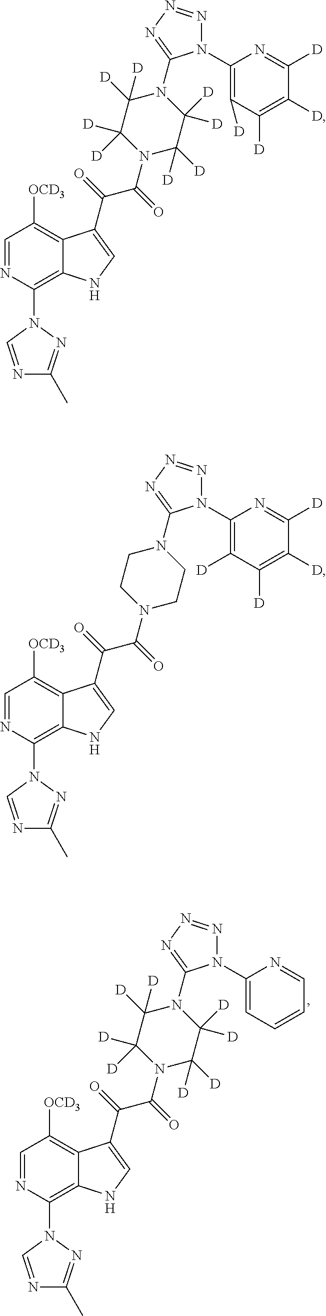

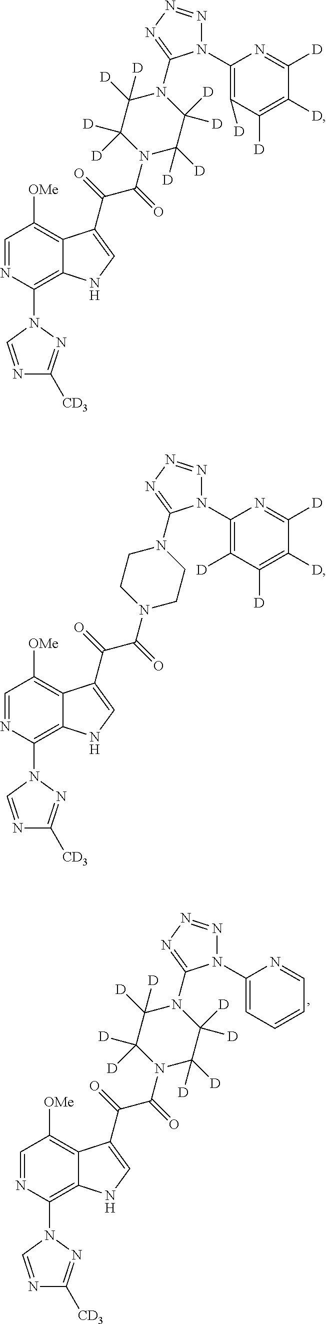

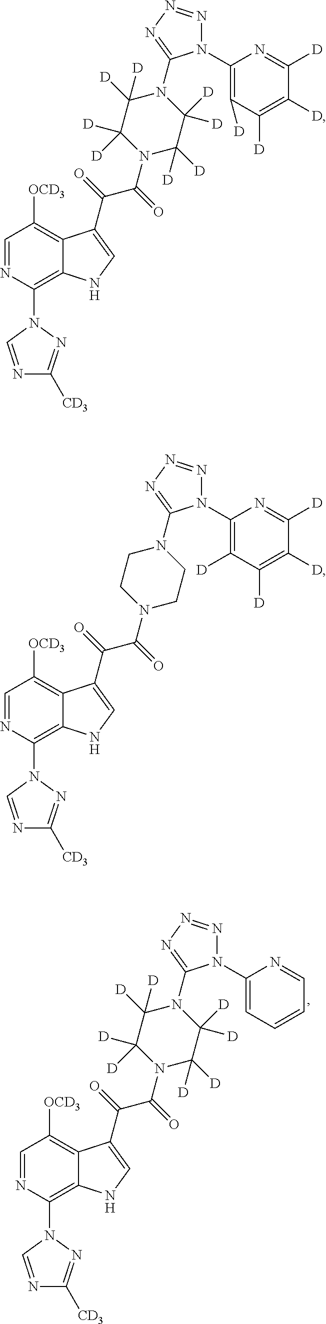

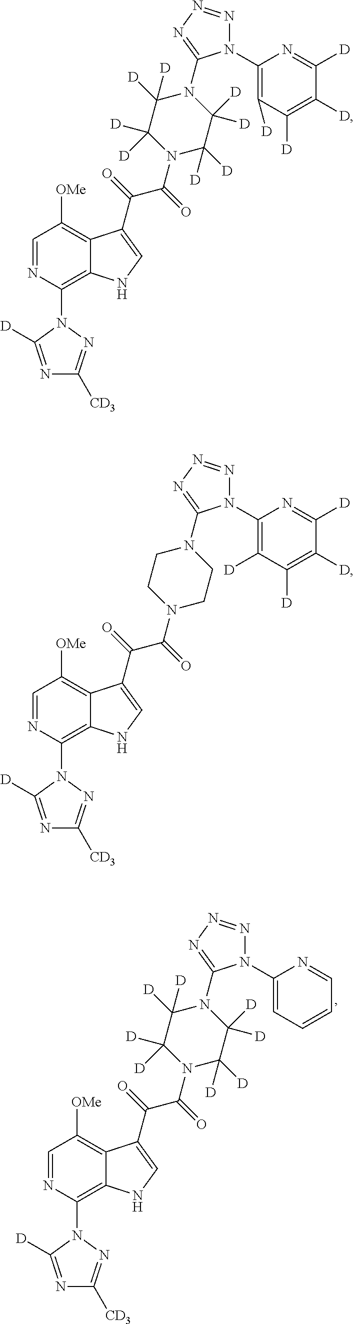

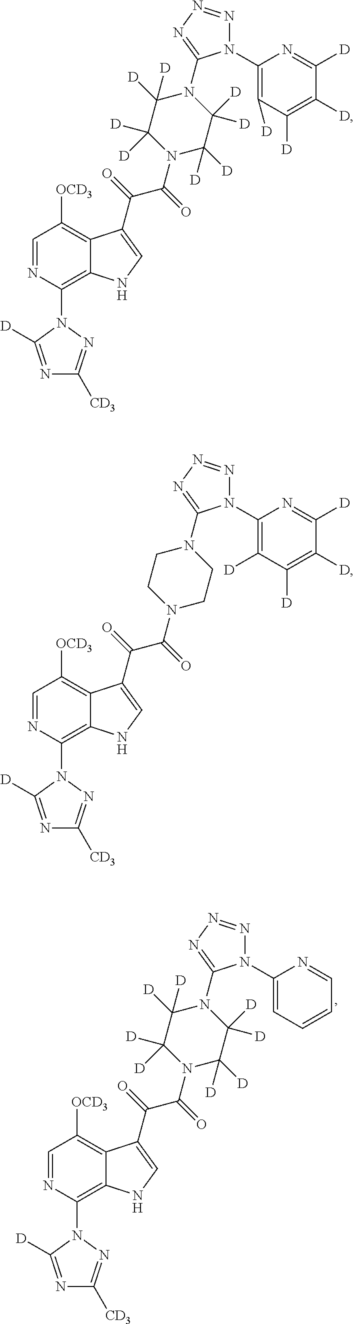

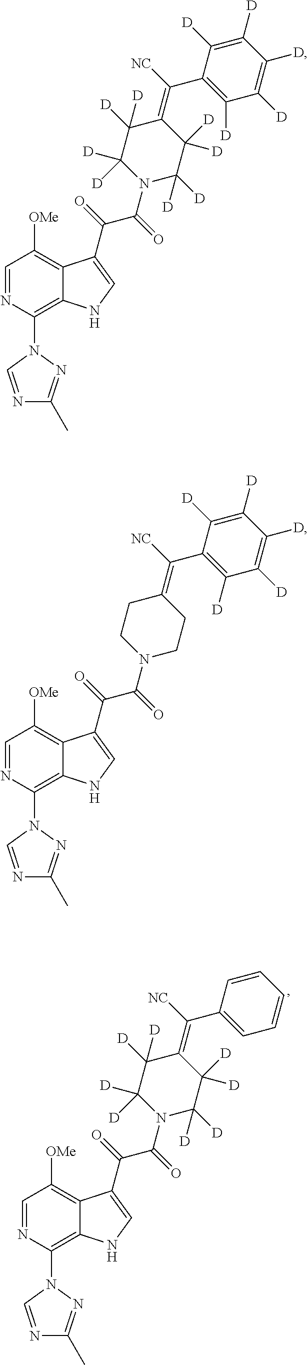

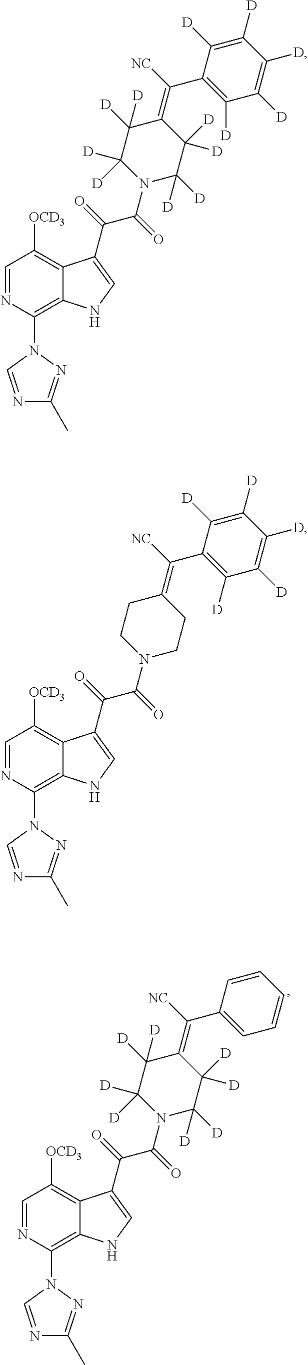

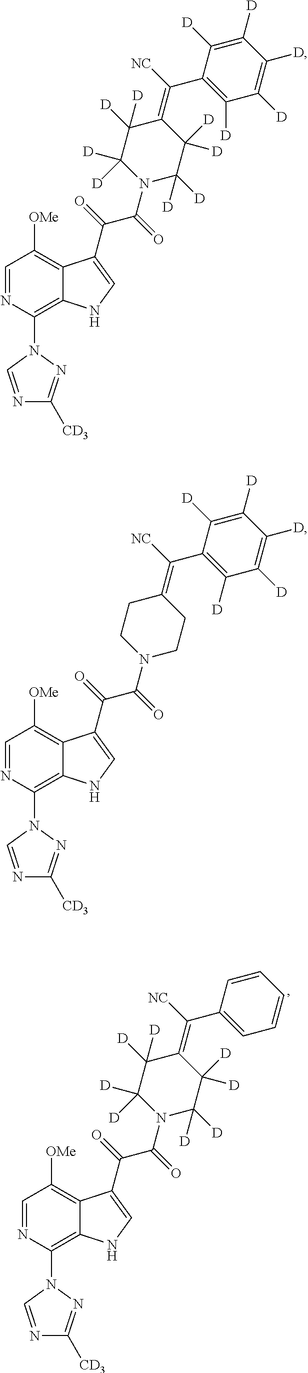

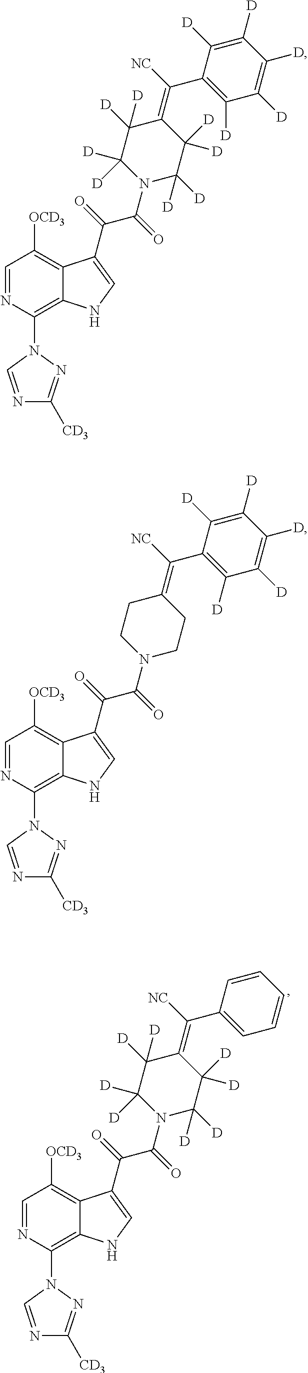

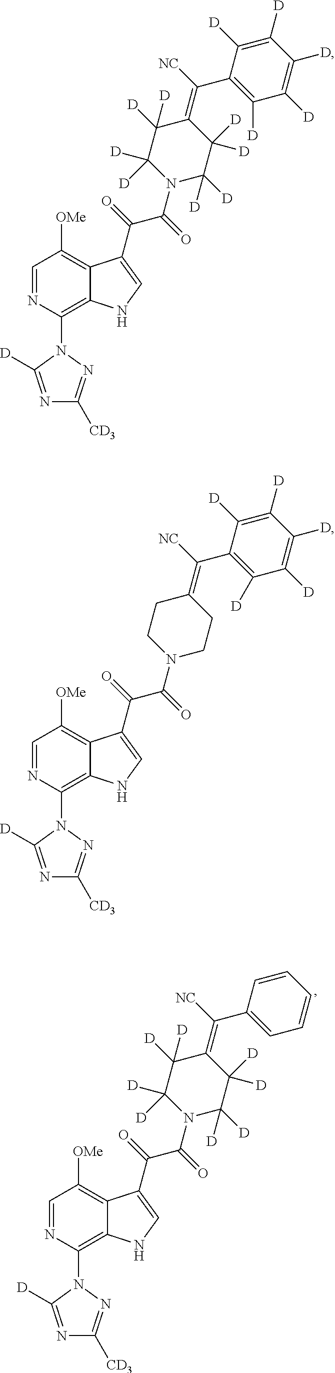

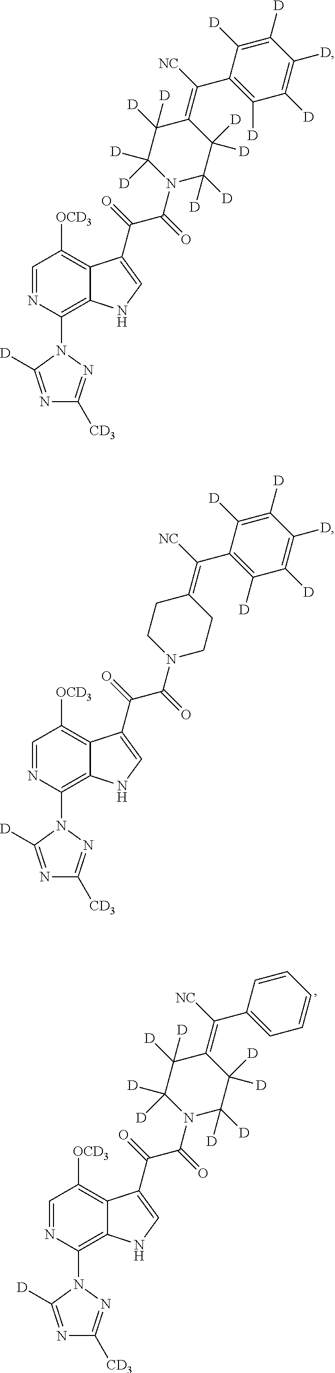

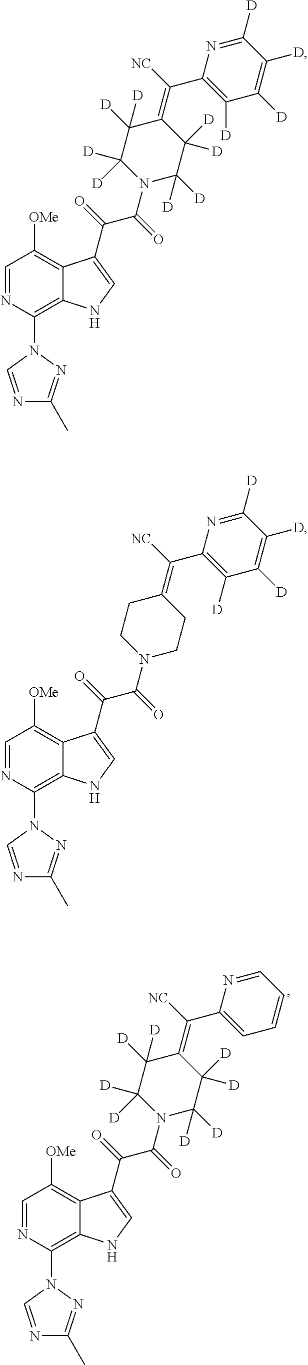

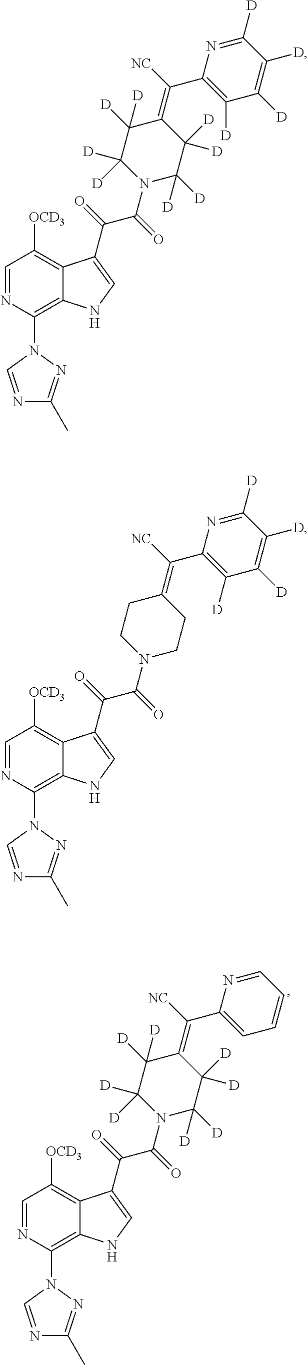

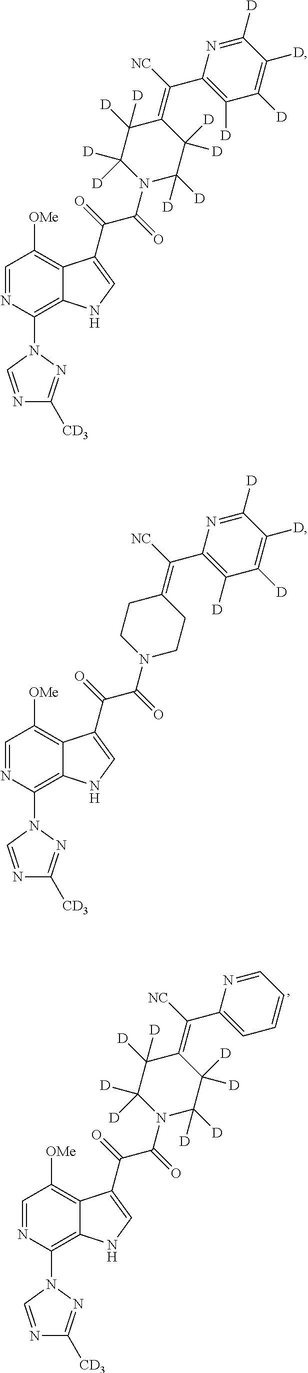

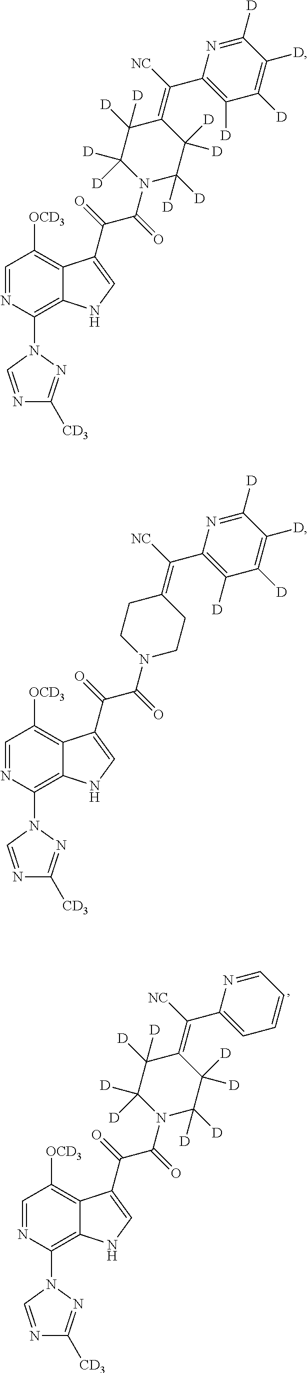

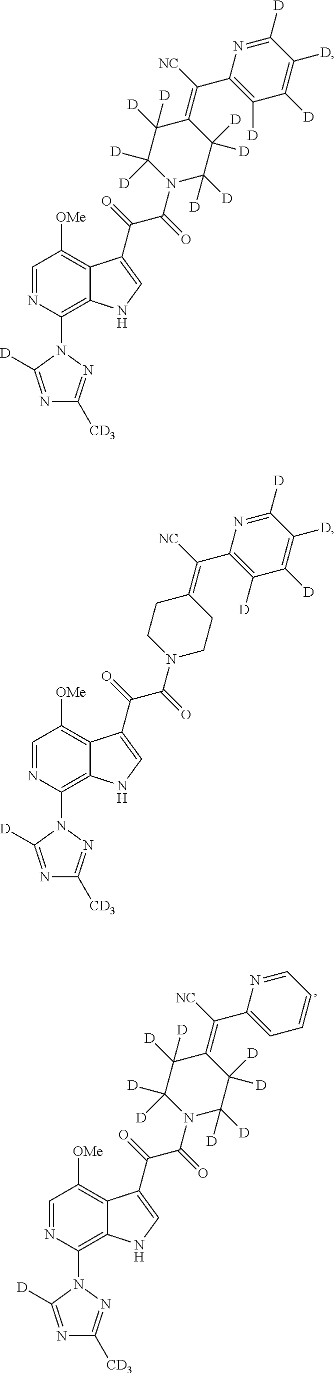

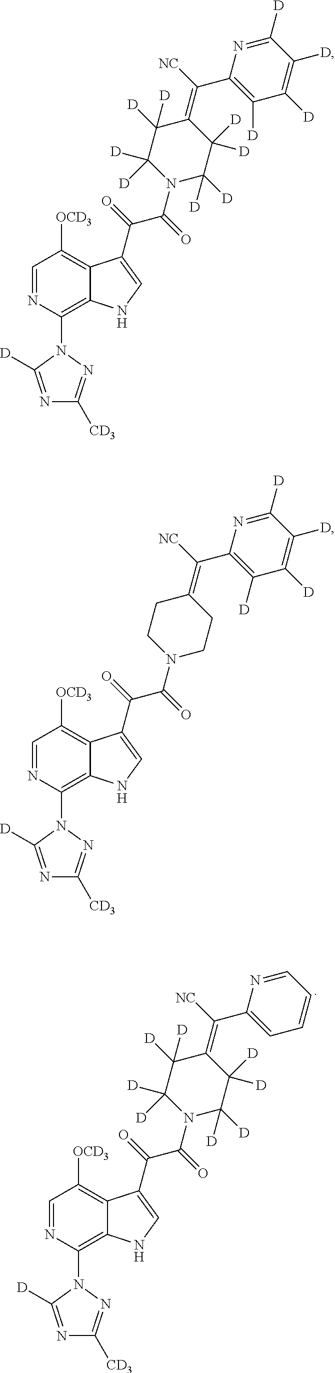

### Chemistry Experimental

### LC/MS Method (i.e., Compound Identification)

All Liquid Chromatography (LC) data were recorded on a Shimadzu LC-10AS or LC-20AS liquid chromotograph using a SPD-10AV or SPD-20A UV-Vis detector and Mass Spectrometry (MS) data were determined with a Micromass Platform for LC in electrospray mode.

### HPLC Method (i.e., Compound Isolation)

Compounds purified by preparative HPLC were diluted in methanol (1.2 mL) and purified using a Shimadzu LC-8A or LC-10A automated preparative HPLC system.

### Intermediate BH:

1) Benzoyl piperazines
Typical procedures were described in application (W. S. Blair, et al. WO-2000076521), by using deuterated starting materials instead.

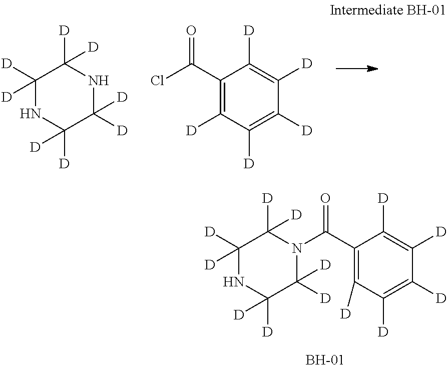

BuLi (3.98 mL, 1.6M in hexane) was added into a solution of piperazine-2,2,3,3,5,5,6,6-D8 (300 mg, from C/D/N Isotopes, Inc., catalog number D-6283) in THF and the resulting mixture was stirred at room temperature for 30 minutes. D5-benzoyl chloride (464 mg, from Sigma-Aldrich, catalog number 366048-1 g) was added to the mixture in one portion. After 5 minutes, the reaction mixture was quenched with MeOH (5 mL). After removal of solvents, the residue was used as is or purified by preparative HPLC.

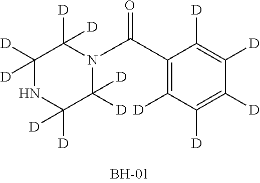 [SMILES: [2H]c1c([2H])c([2H])c(C(=O)N2C([2H])([2H])C([2H])([2H])NC([2H])([2H])C2([2H])[2H])c([2H])c1[2H]]

| | |
|---|---|
| MS (M + H)⁺ Calcd. | 204.2 |
| MS (M + H)⁺ Observ. | 204.2 |
| Retention Time | 1.16 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 2 min |
| Flow Rate | 1 mL/min |
| Wavelength | 220 |
| Solvent Pair | ACN: Water: Ammonium Actetate |
| Column | Phenomenex Luna C18, 30 × 2, 3u |

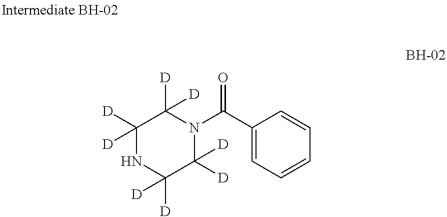 [SMILES: [2H]C1([2H])NC([2H])([2H])C([2H])([2H])N(C(=O)c2ccccc2)C1([2H])[2H]]

Intermediate BH-02 was purchased from C/D/N Isotopes, Inc. (catalog #: D-6285) and used as is.

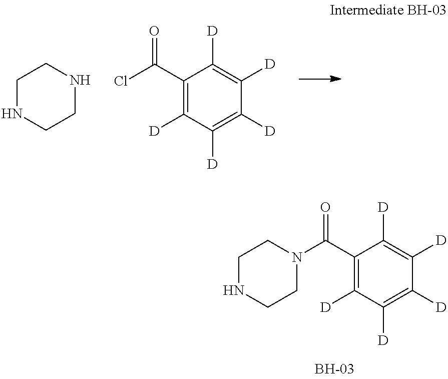 [SMILES: C1CNCCN1 ; [2H]c1c([2H])c([2H])c(C(=O)Cl)c([2H])c1[2H] ; [2H]c1c([2H])c([2H])c(C(=O)N2CCNCC2)c([2H])c1[2H]]

BuLi (4.35 mL, 1.6M in hexane) was added into a solution of piperazine (300 mg) in THF and the resulting mixture was stirred at room temperature for 30 minutes. D5-benzoyl chloride (507 mg) was added to the mixture in one portion. After 5 minutes, the reaction mixture was quenched with MeOH (5 mL). After removal of solvents, the residue was used as is or purified by preparative HPLC.

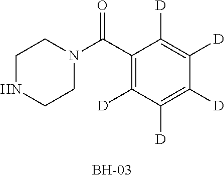 [SMILES: [2H]c1c([2H])c([2H])c(C(=O)N2CCNCC2)c([2H])c1[2H]]

| | |
|---|---|
| MS (M + H)⁺ Calcd. | 196.1 |
| MS (M + H)⁺ Observ. | 196.2 |
| Retention Time | 1.13 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 2 min |
| Flow Rate | 1 mL/min |
| Wavelength | 220 |
| Solvent Pair | ACN: Water: Ammonium Actetate |
| Column | Phenomenex Luna C18, 30 × 2, 3u |

### 2) Phenyl or Pyridyl Tetrazolyl Piperazines

Typical procedures were described in application (T. Wang, et al. US-2007249579) and adapted by using deuterated starting materials instead.

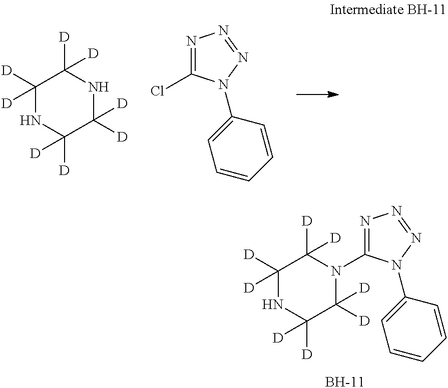

iPr₂NEt (2 mL) was added to a solution of piperazine-2,2,3,3,5,5,6,6-D8 (939 mg) and 5-chloro-1-phenyl-1H-tetrazole (600 mg) in THF (20 mL). The reaction mixture was stirred out at 115° C. for 72 hours before being quenched with water. The aqueous layer was extracted with EtOAc (3×20 mL). The combined organic layer was dried over Mg₂SO₄ and concentrated to offer a residue which was used without purification.

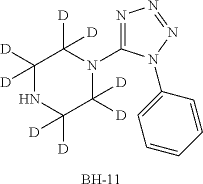

| | |
|---|---|
| MS (M + H)⁺ Calcd. | 239.2 |
| MS (M + H)⁺ Observ. | 239.2 |
| Retention Time | 1.24 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 2 min |
| Flow Rate | 1 mL/min |
| Wavelength | 220 |
| Solvent Pair | ACN: Water: Ammonium Actetate |
| Column | Phenomenex Luna C18, 30 × 2, 3u |

### 3) Cyano Phenyl or Pyridyl Alkenyl Piperidines

Typical procedures were described in application (T. Wang, et al. US-2004063744), by using deuterated starting materials instead.

### Intermediate ACOCOOH:

Preparation of intermediate ACOCOOH was described in the previous published applications (T. Wang, et al. WO-2001062255 and T. Wang, et al. WO-2002062423). Some examples of ACOCOOH are listed in below.

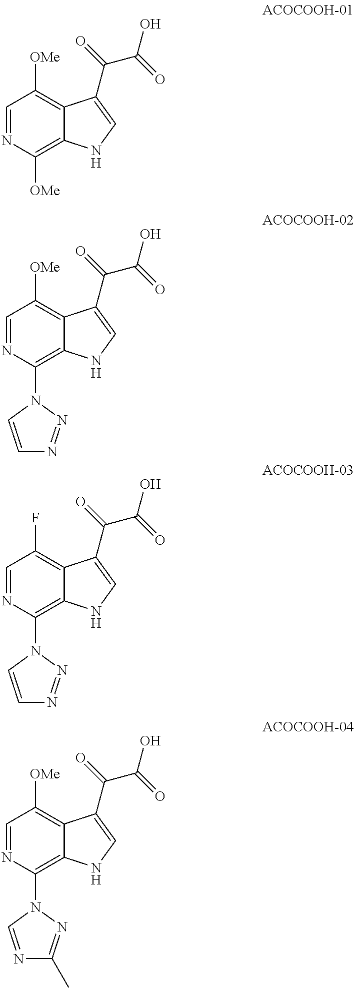 [SMILES: COc1ncc(OC)c2c(C(=O)C(=O)O)c[nH]c12 ; COc1cnc(-n2ccnn2)c2[nH]cc(C(=O)C(=O)O)c12 ; O=C(O)C(=O)c1c[nH]c2c(-n3ccnn3)ncc(F)c12 ; COc1cnc(-n2cnc(C)n2)c2[nH]cc(C(=O)C(=O)O)c12]

### Syntheses of the Compounds of Formula I

Typical procedures were described in application (T. Wang, et al. WO-2001062255, T. Wang, et al. WO-2002062423, T. Wang, et al. US-2007249579 and T. Wang, et al. U.S. Pat. No. 2,004,063744), by using deuterated starting materials instead.
General procedure to prepare compounds 0001 to 0012.

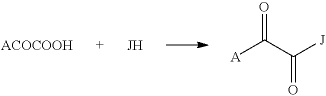

2-Keto acid (1 eq.), deuterated benzoyl piperazine (1-5 eq.), 3-(diethoxyphosphoryloxy)-1,2,3-benzotriazin-4(3H)-one (DEPBT) or O-(1H-benzotriazol-1-yl)-N,N,N′,N′-tetramethyluronium tetrafluoroborate (TBTU) (1-5 eq.) or (2-(7-Aza-1H-benzotriazole-1-yl)-1,1,3,3-tetramethyluronium hexafluorophosphate) (HATU) (1-5 eq.) and Hunig's Base or N-methyl morpholine (1-100 eq.) were combined in THF or DMF. The mixture was stirred at room temperature or 115° C. for 17 hours. THF or DMF was removed via evaporation at reduced pressure and the residue was partitioned between ethyl acetate and saturated NaHCO₃ aqueous solution. The aqueous layer was extracted with ethyl acetate. The organic phase was combined and dried over anhydrous MgSO₄. Concentration in vacuo provided a crude product, which was purified by tritaration, or recrystallization, or silica gel column chromatography, or Shimadzu automated preparative HPLC System.

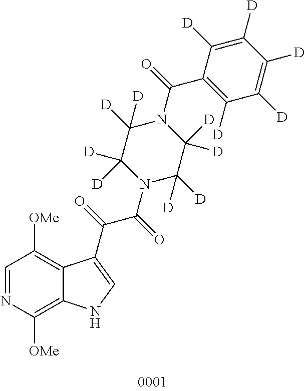 [SMILES: [2H]c1c([2H])c([2H])c(C(=O)N2C([2H])([2H])C([2H])([2H])N(C(=O)C(=O)c3c[nH]c4c(OC)ncc(OC)c34)C([2H])([2H])C2([2H])[2H])c([2H])c1[2H]]

Compound 0001 was prepared by coupling ACOCOOH-01 and
BH-01 by following general procedure.

| | |
|---|---|
| MS (M + H)⁺ Calcd. | 436.2 |
| MS (M + H)⁺ Observ. | 436.1 |
| Retention Time | 3.41 min |

LC Condition

| | |
|---|---|
| Solvent A | 90% Water-0% Methanol-0.1% TFA |
| Solvent B | 10% Water-90% Methanol-0.1% TFA |
| Start % B | 0 |
| Final % B | 100 |
| Gradient Time | 4 min |
| Flow Rate | 0.8 mL/min |
| Wavelength | 220 |
| Solvent Pair | Water-Methanol-TFA |
| Column | PHENOMENEX-LUNA 2.0 × 50 mm 3 um |

NMR

| | |
|---|---|
| ¹H (500 MHz, | 8.31 (s, 1H), 7.42 (s, 1H), 4.20 (s, 3H), |
| CD₃OD) δ ppm | 3.97 (s, 3H) |

HRMS

| | |
|---|---|
| MS (M + H)⁺ Calcd. for | 436.2484 |
| C₂₂H₁₀D₁₃N₄O₅ | |
| MS (M + H)⁺ Observ. | 436.2468 |

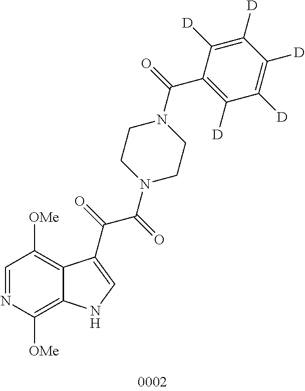 [SMILES: [2H]c1c([2H])c([2H])c(C(=O)N2CCN(C(=O)C(=O)c3c[nH]c4c(OC)ncc(OC)c34)CC2)c([2H])c1[2H]]

Compound 0002 was prepared by coupling ACOCOOH-01 and
BH-02 by following general procedure.

| | |
|---|---|
| MS (M + H)⁺ Calcd. | 428.2 |
| MS (M + H)⁺ Observ. | 428.1 |
| Retention Time | 3.42 min |

LC Condition

| | |
|---|---|
| Solvent A | 90% Water-0% Methanol-0.1% TFA |
| Solvent B | 10% Water-90% Methanol-0.1% TFA |
| Start % B | 0 |
| Final % B | 100 |
| Gradient Time | 4 min |
| Flow Rate | 0.8 mL/min |
| Wavelength | 220 |
| Solvent Pair | Water-Methanol-TFA |
| Column | PHENOMENEX-LUNA 2.0 × 50 mm 3 um |

NMR

| | |
|---|---|
| ¹H (500 MHz, | 8.31 (s, 1H), 7.43 (s, 1H), 4.20 (s, 3H), 3.97 |
| CD₃OD) δ ppm | (s, 3H), 4.00-3.40 (m, 8H) |

HRMS

| | |
|---|---|
| MS (M + H)⁺ Calcd. for | 428.1982 |
| C₂₂H₁₈D₅N₄O₅ | |
| MS (M + H)⁺ Observ. | 428.1970 |

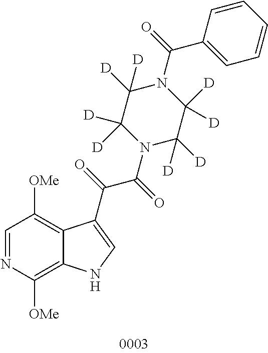 [SMILES: [2H]C1([2H])N(C(=O)C(=O)c2c[nH]c3c(OC)ncc(OC)c23)C([2H])([2H])C([2H])([2H])N(C(=O)c2ccccc2)C1([2H])[2H]]

Compound 0003 was prepared by coupling ACOCOOH-01 and
BH-03 by following general procedure.

| | |
|---|---|
| MS (M + H)⁺ Calcd. | 431.2 |
| MS (M + H)⁺ Observ. | 431.1 |
| Retention Time | 3.45 min |

LC Condition

| | |
|---|---|
| Solvent A | 90% Water-10% Methanol-0.1% TFA |
| Solvent B | 10% Water-90% Methanol-0.1% TFA |
| Start % B | 0 |
| Final % B | 100 |
| Gradient Time | 4 min |
| Flow Rate | 0.8 mL/min |
| Wavelength | 220 |
| Solvent Pair | Water-Methanol-TFA |
| Column | PHENOMENEX-LUNA 2.0 × 50 mm 3 um |

NMR

| | |
|---|---|
| ¹H (500 MHz, | 8.31 (s, 1H), 7.49 (b, 5H), 7.43 (s, 1H), |
| CD₃OD) δ ppm | 4.20 (s, 3H), 3.97 (s, 3H) |

HRMS

| | |
|---|---|
| MS (M + H)⁺ Calcd. for | 431.2171 |
| C₂₂H₁₅D₈N₄O₅ | |
| MS (M + H)⁺ Observ. | 431.2158 |

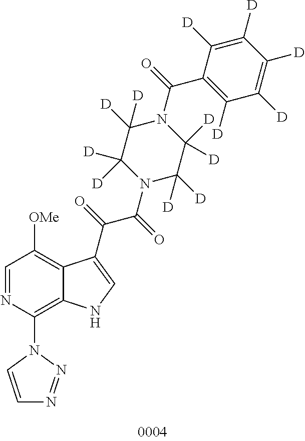 [SMILES: [2H]c1c([2H])c([2H])c(C(=O)N2C([2H])([2H])C([2H])([2H])N(C(=O)C(=O)c3c[nH]c4c(-n5ccnn5)ncc(OC)c34)C([2H])([2H])C2([2H])[2H])c([2H])c1[2H]]

Compound 0004 was prepared by coupling ACOCOOH-02 and
BH-01 by following general procedure.

| | |
|---|---|
| MS (M + H)⁺ Calcd. | 473.3 |
| MS (M + H)⁺ Observ. | 473.0 |
| Retention Time | 1.75 min |

LC Condition

| | |
|---|---|
| Solvent A | 90% Water-10% Methanol-0.1% TFA |
| Solvent B | 10% Water-90% Methanol-0.1% TFA |
| Start % B | 0 |
| Final % B | 100 |
| Gradient Time | 2 min |
| Flow Rate | 1 mL/min |
| Wavelength | 220 |
| Solvent Pair | Water-Methanol-TFA |
| Column | PHENOMENEX-LUNA 2.0 × 30 mm 3 um |

NMR

| | |
|---|---|
| ¹H (500 MHz, | 8.79 (s, 1H), 8.33 (s, 1H), 7.93 (s, 1H), |
| CD₃OD) δ ppm | 7.90 (s, 1H), 4.10 (s, 3H) |

HRMS

| | |
|---|---|
| MS (M + H)⁺ Calcd. for | 473.2549 |
| C₂₃H₉D₁₃N₇O₄ | |
| MS (M + H)⁺ Observ. | 473.2542 |

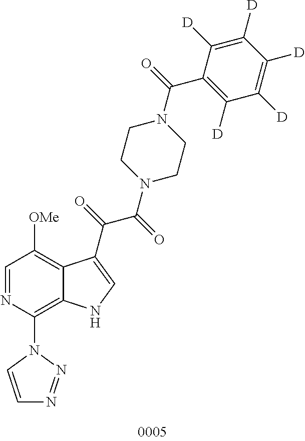 [SMILES: [2H]c1c([2H])c([2H])c(C(=O)N2CCN(C(=O)C(=O)c3c[nH]c4c(-n5ccnn5)ncc(OC)c34)CC2)c([2H])c1[2H]]

Compound 0005 was prepared by coupling ACOCOOH-02 and
BH-02 by following general procedure.

| | |
|---|---|
| MS (M + H)⁺ Calcd. | 465.2 |
| MS (M + H)⁺ Observ. | 465.3 |
| Retention Time | 2.06 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 4 min |
| Flow Rate | 0.8 mL/min |
| Wavelength | 220 |
| Solvent Pair | ACN: Water: Ammonium Actetate |
| Column | Phenomenex LUNA C18, 50 × 2, 3 u |

NMR

| | |
|---|---|
| ¹H (500 MHz, | 12.81 (s, 1H), 8.96 (s, 1H), 8.25 (s, 1H), 8.10 |
| DMSO-D₆) δ ppm | (s, 1H), 8.00 (s, 1H), 4.03 (s, 3H), 3.90-3.30 |
| | (m, 8H) |

HRMS

| | |
|---|---|
| MS (M + H)⁺ Calcd. for | 465.2047 |
| C₂₃H₁₇D₅N₇O₄ | |
| MS (M + H)⁺ Observ. | 465.2036 |

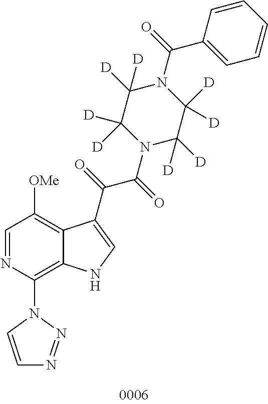 [SMILES: [2H]C1([2H])N(C(=O)C(=O)c2c[nH]c3c(-n4ccnn4)ncc(OC)c23)C([2H])([2H])C([2H])([2H])N(C(=O)c2ccccc2)C1([2H])[2H]]

Compound 0006 was prepared by coupling ACOCOOH-02 and
BH-03 by following general procedure.

| | |
|---|---|
| MS (M + H)⁺ Calcd. | 468.2 |
| MS (M + H)⁺ Observ. | 468.3 |
| Retention Time | 1.30 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 2 min |
| Flow Rate | 1 mL/min |
| Wavelength | 220 |
| Solvent Pair | ACN: Water: Ammonium Actetate |
| Column | Phenomenex LUNA C18, 30 × 2, 3 u |

NMR

| | |
|---|---|
| ¹H (500 MHz, | 12.81 (s, 1H), 8.96 (s, 1H), 8.26 (s, 1H), 8.10 |
| DMSO-D₆) δ ppm | (s, 1H), 8.00 (s, 1H), 7.47 (b, 5H), 4.05 (s, 3H) |

HRMS

| | |
|---|---|
| MS (M + H)⁺ Calcd. for | 468.2235 |
| C₂₃H₁₄D₈N₇O₄ | |
| MS (M + H)⁺ Observ. | 468.2224 |

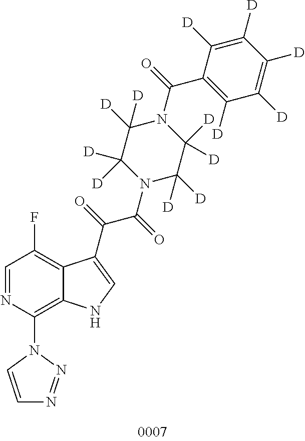 [SMILES: [2H]c1c([2H])c([2H])c(C(=O)N2C([2H])([2H])C([2H])([2H])N(C(=O)C(=O)c3c[nH]c4c(-n5ccnn5)ncc(F)c34)C([2H])([2H])C2([2H])[2H])c([2H])c1[2H]]

Compound 0007 was prepared by coupling ACOCOOH-03 and
BH-01 by following general procedure.

| | |
|---|---|
| MS (M + H)⁺ Calcd. | 461.2 |
| MS (M + H)⁺ Observ. | 461.0 |
| Retention Time | 1.85 min |

LC Condition

| | |
|---|---|
| Solvent A | 90% Water-10% Methanol-0.1% TFA |
| Solvent B | 10% Water-90% Methanol-0.1% TFA |
| Start % B | 0 |
| Final % B | 100 |
| Gradient Time | 2 min |
| Flow Rate | 1 mL/min |
| Wavelength | 220 |
| Solvent Pair | Water-Methanol-TFA |
| Column | PHENOMENEX-LUNA 2.0 × 30 mm 3 um |

NMR

| | |
|---|---|
| ¹H (500 MHz, | 9.03 (s, 1H), 8.39 (s, 1H), 8.34 (s, 1H), |
| DMSO-D₆) δ ppm | 8.14 (s, 1H) |

HRMS

| | |
|---|---|
| MS (M + H)⁺ Calcd. for | 461.2349 |
| C₂₂H₆D₁₃FN₇O₃ | |
| MS (M + H)⁺ Observ. | 461.2338 |

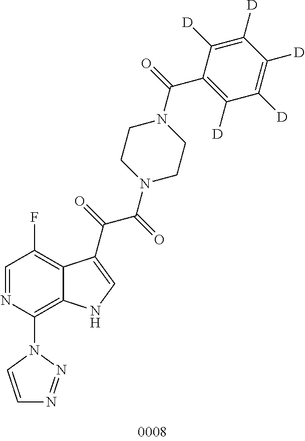 [SMILES: [2H]c1c([2H])c([2H])c(C(=O)N2CCN(C(=O)C(=O)c3c[nH]c4c(-n5ccnn5)ncc(F)c34)CC2)c([2H])c1[2H]]

Compound 0008 was prepared by coupling ACOCOOH-03 and
BH-02 by following general procedure.

| | |
|---|---|
| MS (M + H)⁺ Calcd. | 453.2 |
| MS (M + H)⁺ Observ. | 453.3 |
| Retention Time | 1.49 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 2 min |
| Flow Rate | 1 mL/min |
| Wavelength | 220 |
| Solvent Pair | ACN: Water: Ammonium Actetate |
| Column | Phenomenex LUNA C18, 30 × 2, 3 u |

NMR

| | |
|---|---|
| ¹H (500 MHz, | 9.02 (s, 1H), 8.40 (s, 1H), 8.33 (s, 1H), |
| DMSO-D₆) δ ppm | 8.14 (s, 1H), 4.00-3.30 (m, 8H) |

HRMS

| | |
|---|---|
| MS (M + H)⁺ Calcd. for | 453.1847 |
| C₂₂H₁₄D₅FN₇O₃ | |
| MS (M + H)⁺ Observ. | 453.1838 |

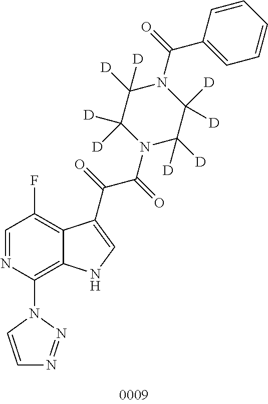 [SMILES: [2H]C1([2H])N(C(=O)C(=O)c2c[nH]c3c(-n4ccnn4)ncc(F)c23)C([2H])([2H])C([2H])([2H])N(C(=O)c2ccccc2)C1([2H])[2H]]

Compound 0009 was prepared by coupling ACOCOOH-03 and
BH-03 by following general procedure.

| | |
|---|---|
| MS (M + H)⁺ Calcd. | 456.2 |
| MS (M + H)⁺ Observ. | 456.3 |
| Retention Time | 2.21 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 4 min |
| Flow Rate | 0.8 mL/min |
| Wavelength | 220 |
| Solvent Pair | ACN: Water: Ammonium Actetate |
| Column | Phenomenex LUNA C18, 50 × 2, 3 u |

NMR

| | |
|---|---|
| ¹H (500 MHz, | 9.03 (s, 1H), 8.39 (s, 1H), 8.32 (s, 1H), 8.14 |
| DMSO-D₆) δ ppm | (s, 1H), 7.46 (b, 5H) |

HRMS

| | |
|---|---|
| MS (M + H)⁺ Calcd. for | 456.2036 |
| C₂₂H₁₁D₈FN₇O₃ | |
| MS (M + H)⁺ Observ. | 456.2027 |

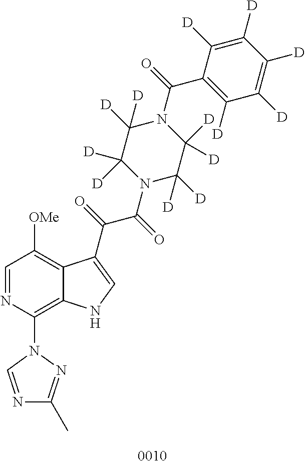 [SMILES: [2H]c1c([2H])c([2H])c(C(=O)N2C([2H])([2H])C([2H])([2H])N(C(=O)C(=O)c3c[nH]c4c(-n5cnc(C)n5)ncc(OC)c34)C([2H])([2H])C2([2H])[2H])c([2H])c1[2H]]

Compound 0010 was prepared by coupling ACOCOOH-04 and
BH-01 by following general procedure.

| | |
|---|---|
| MS (M + H)⁺ Calcd. | 487.3 |
| MS (M + H)⁺ Observ. | 487.1 |
| Retention Time | 1.76 min |

LC Condition

| | |
|---|---|
| Solvent A | 90% Water-10% Methanol-0.1% TFA |
| Solvent B | 10% Water-90% Methanol-0.1% TFA |
| Start % B | 0 |
| Final % B | 100 |
| Gradient Time | 2 min |
| Flow Rate | 1 mL/min |
| Wavelength | 220 |
| Solvent Pair | Water-Methanol-TFA |
| Column | PHENOMENEX-LUNA 2.0 × 30 mm 3 um |

NMR

| | |
|---|---|
| ¹H (500 MHz, | 9.16 (s, 1H), 8.30 (s, 1H), 7.81 (s, 1H), 4.07 |
| CD₃OD-CDCl₃) δ pps | (s, 3H), 2.56 (s, 3H) |

HRMS

| | |
|---|---|
| MS (M + H)⁺ Calcd. for | 487.2706 |
| C₂₄H₁₁D₁₃N₇O₄ | |
| MS (M + H)⁺ Observ. | 487.2695 |

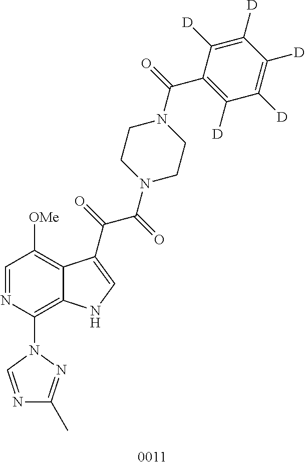 [SMILES: [2H]c1c([2H])c([2H])c(C(=O)N2CCN(C(=O)C(=O)c3c[nH]c4c(-n5cnc(C)n5)ncc(OC)c34)CC2)c([2H])c1[2H]]

Compound 0011 was prepared by coupling ACOCOOH-04 and
BH-02 by following general procedure.

| | |
|---|---|
| MS (M + H)⁺ Calcd. | 479.2 |
| MS (M + H)⁺ Observ. | 479.3 |
| Retention Time | 2.13 min |

LC Condition

| | |
|---|---|
| Solvent A | 5% ACN:95% Water: 10 mM Ammonium |
| | Actetate |
| Solvent B | 95% ACN:5% Water: 10 mM Ammonium |
| | Actetate |
| Start % B | 0 |
| Final % B | 100 |
| Gradient Time | 4 min |
| Flow Rate | 0.8 mL/min |
| Wavelength | 220 |
| Solvent Pair | ACN: Water: Ammonium Actetate |
| Column | Phenomenex LUNA C18, 50 × 2, 3 u |

NMR

| | |
|---|---|
| ¹H (500 MHz, | 12.42 (s, 1H), 9.26 (s, 1H), 8.26 (s, 1H), |
| DMSO-D₆) δ ppm | 7.91 (s, 1H), 4.01 (s, 3H), 3.90-3.30 |
| | (m, 8H), 2.52 (s, 3H) |

HRMS

| | |
|---|---|
| MS (M + H)⁺ Calcd. for | 479.2204 |
| C₂₄H₁₉D₅N₇O₄ | |
| MS (M + H)⁺ Observ. | 479.2195 |

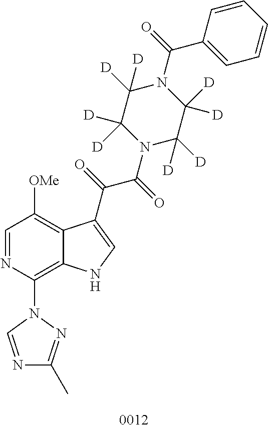 [SMILES: [2H]C1([2H])N(C(=O)C(=O)c2c[nH]c3c(-n4cnc(C)n4)ncc(OC)c23)C([2H])([2H])C([2H])([2H])N(C(=O)c2ccccc2)C1([2H])[2H]]

Compound 0012 was prepared by coupling ACOCOOH-04 and
BH-03 by following general procedure.

| | |
|---|---|
| MS (M + H)⁺ Calcd. | 482.2 |
| MS (M + H)⁺ Observ. | 482.1 |
| Retention Time | 1.76 min |

LC Condition

| | |
|---|---|
| Solvent A | 90% Water-10% Methanol-0.1% TFA |
| Solvent B | 10% Water-90% Methanol-0.1% TFA |
| Start % B | 0 |
| Final % B | 100 |
| Gradient Time | 2 min |
| Flow Rate | 1 mL/min |
| Wavelength | 220 |
| Solvent Pair | Water-Methanol-TFA |
| Column | PHENOMENEX-LUNA 2.0 × 30 mm 3 um |

NMR

| | |
|---|---|
| ¹H (500 MHz, | 12.42 (s, 1H), 9.26 (s, 1H,), 8.27 (s, 1H), 7.91 |
| DMSO-D₆) δ ppm | (s, 1H), 7.48 (b, 5H), 4.01 (s, 3H), 2.52 (s, 3H) |

HRMS

| | |
|---|---|
| MS (M + H)⁺ Calcd. for | 482.2392 |
| C₂₄H₁₆D₈N₇O₄ | |
| MS (M + H)⁺ Observ. | 482.2379 |

### Biology Data for the Examples

“μM” means micromolar;
“mL” means milliliter;
“μl” means microliter;
“mg” means milligram;

The materials and experimental procedures used to obtain the results reported in Table 1 are described below.

### Cells:

Virus production—Human embryonic Kidney cell line, 293T (HEK 293T), was propagated in Dulbecco's Modified Eagle Medium (Invitrogen, Carlsbad, Calif.) containing 10% fetal Bovine serum (FBS, Sigma, St. Louis, Mo.). The human T-cell leukemia cell MT2 (AIDS Research and Reference Reagent Program, Cat. 237) was propagated in RPMI 1640 (Invitrogen, Carlsbad, Calif.) containing 10% fetal bovine serum (FBS, Hyclone, Logan, Utah)
Virus infection—Single-round infectious reporter virus was produced by co-transfecting HEK 293T cells with plasmide expressing the HIV-1 LAI envelope along with a plasmid containing an HIV-1 LAI proviral cDNA with the envelope gene replaced by a firefly luciferase reporter gene (Chen et al, Ref 41). Transfections were performed using lipofectAMINE PLUS reagent as described by the manufacturer (Invitrogen, Carlsbad, Calif.).

### Experimental Procedure

1. MT2 cells were plated in black, 384 well plates at a cell density of 5×10³ cells per well in 25 μl RPMI 1640 containing 10% FBS.
2. Compound (diluted in dimethylsulfoxide and growth medium) was added to cells at 12.5 μl/well, so that the final assay concentration would be ≦50 nM.
3. 12.5 μl of single-round infectious reporter virus in Dulbecco's Modified Eagle Medium was added to the plated cells and compound at an approximate multiplicity of infection (MOI) of 0.01, resulting in a final volume of 50 μl per well.
4. Virus-infected cells were incubated at 37 degrees Celsius, in a CO₂ incubator, and harvested 72 h after infection.
5. Viral infection was monitored by measuring luciferase expression in the infected cells using a luciferase reporter gene assay kit (Steady-Glo, Promega, Madison, Wis.) as described by the manufacturer. Luciferase activity was then quantified by measuring luminescence using an EnVision Multilabel Plate Readers (PerkinElmer, Waltham, Mass.).
6. The percent inhibition for each compound was calculated by quantifying the level of luciferase expression in cells infected in the presence of each compound as a percentage of that observed for cells infected in the absence of compound and subtracting such a determined value from 100.
7. An EC₅₀ provides a method for comparing the antiviral potency of the compounds of this disclosure. The effective concentration for fifty percent inhibition (EC₅₀) was calculated with the Microsoft Excel Xlfit curve fitting software. For each compound, curves were generated from percent inhibition calculated at 10 different concentrations by using a four paramenter logistic model (model 205). The EC₅₀ data for the compounds is shown in Table 1.

TABLE 1

| Compound | | EC₅₀ |
|---|---|---|
| # | Structure | (nM) |
| 0001 | 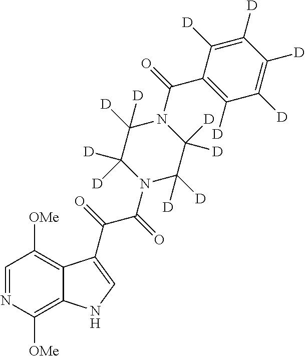 [SMILES: [2H]c1c([2H])c([2H])c(C(=O)N2C([2H])([2H])C([2H])([2H])N(C(=O)C(=O)c3c[nH]c4c(OC)ncc(OC)c34)C([2H])([2H])C2([2H])[2H])c([2H])c1[2H]] | 0.67 |
| 0002 | 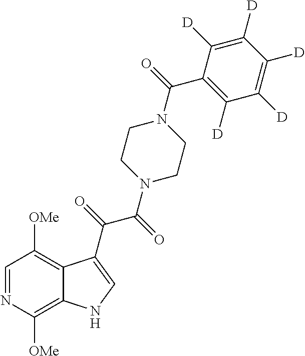 [SMILES: [2H]c1c([2H])c([2H])c(C(=O)N2CCN(C(=O)C(=O)c3c[nH]c4c(OC)ncc(OC)c34)CC2)c([2H])c1[2H]] | 0.63 |
| 0003 | 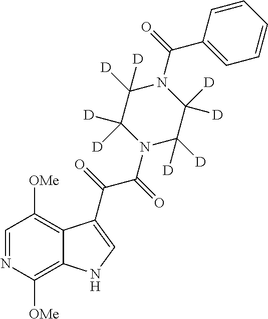 [SMILES: [2H]C1([2H])N(C(=O)C(=O)c2c[nH]c3c(OC)ncc(OC)c23)C([2H])([2H])C([2H])([2H])N(C(=O)c2ccccc2)C1([2H])[2H]] | 0.55 |
| 0004 | 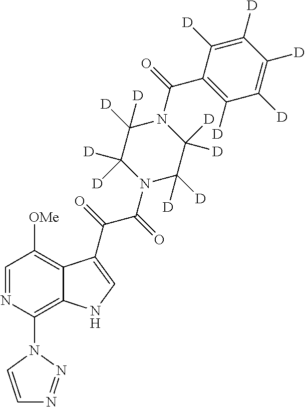 [SMILES: [2H]c1c([2H])c([2H])c(C(=O)N2C([2H])([2H])C([2H])([2H])N(C(=O)C(=O)c3c[nH]c4c(-n5ccnn5)ncc(OC)c34)C([2H])([2H])C2([2H])[2H])c([2H])c1[2H]] | 0.10 |
| 0005 | 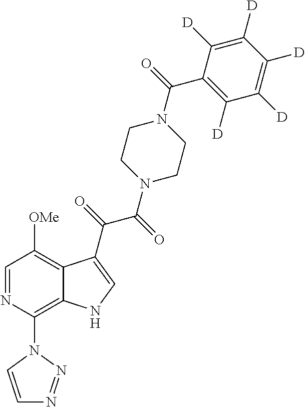 [SMILES: [2H]c1c([2H])c([2H])c(C(=O)N2CCN(C(=O)C(=O)c3c[nH]c4c(-n5ccnn5)ncc(OC)c34)CC2)c([2H])c1[2H]] | 0.24 |
| 0006 | 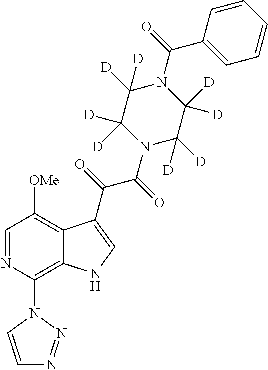 [SMILES: [2H]C1([2H])N(C(=O)C(=O)c2c[nH]c3c(-n4ccnn4)ncc(OC)c23)C([2H])([2H])C([2H])([2H])N(C(=O)c2ccccc2)C1([2H])[2H]] | 0.08 |
| 0007 | 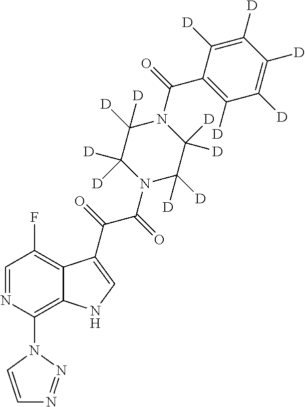 [SMILES: [2H]c1c([2H])c([2H])c(C(=O)N2C([2H])([2H])C([2H])([2H])N(C(=O)C(=O)c3c[nH]c4c(-n5ccnn5)ncc(F)c34)C([2H])([2H])C2([2H])[2H])c([2H])c1[2H]] | 0.11 |
| 0008 | 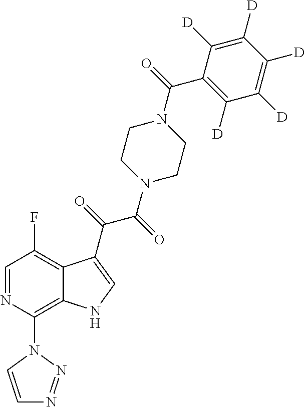 [SMILES: [2H]c1c([2H])c([2H])c(C(=O)N2CCN(C(=O)C(=O)c3c[nH]c4c(-n5ccnn5)ncc(F)c34)CC2)c([2H])c1[2H]] | 0.10 |
| 0009 | 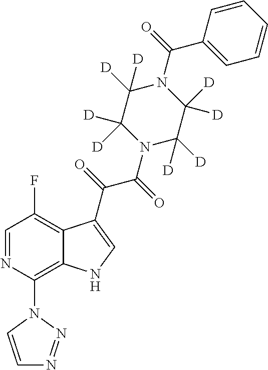 [SMILES: [2H]C1([2H])N(C(=O)C(=O)c2c[nH]c3c(-n4ccnn4)ncc(F)c23)C([2H])([2H])C([2H])([2H])N(C(=O)c2ccccc2)C1([2H])[2H]] | 0.14 |
| 0010 | 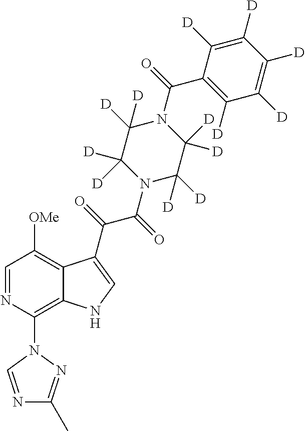 [SMILES: [2H]c1c([2H])c([2H])c(C(=O)N2C([2H])([2H])C([2H])([2H])N(C(=O)C(=O)c3c[nH]c4c(-n5cnc(C)n5)ncc(OC)c34)C([2H])([2H])C2([2H])[2H])c([2H])c1[2H]] | 0.06 |
| 0011 | 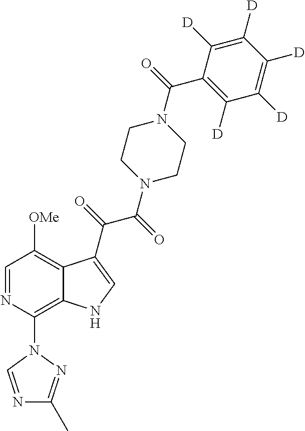 [SMILES: [2H]c1c([2H])c([2H])c(C(=O)N2CCN(C(=O)C(=O)c3c[nH]c4c(-n5cnc(C)n5)ncc(OC)c34)CC2)c([2H])c1[2H]] | 0.12 |
| 0012 | 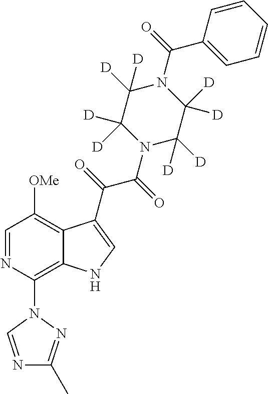 [SMILES: [2H]C1([2H])N(C(=O)C(=O)c2c[nH]c3c(-n4cnc(C)n4)ncc(OC)c23)C([2H])([2H])C([2H])([2H])N(C(=O)c2ccccc2)C1([2H])[2H]] | 0.07 |

What is claimed is:

1. A compound of Formula I, including pharmaceutically acceptable salts thereof: 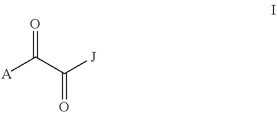
wherein A is selected from the group consisting of: 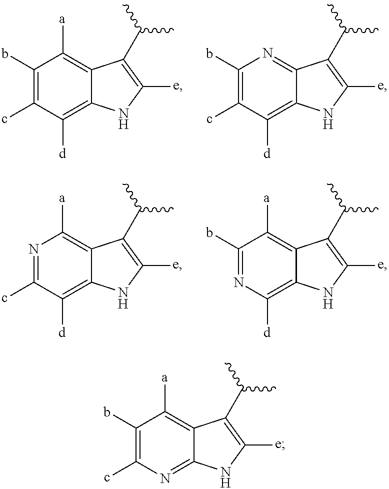
wherein
a, b, c, d and e are independently selected from the group consisting of hydrogen, halogen, cyano, nitro, COOR⁵⁶, XR⁵⁷, C(O)R⁷, C(O)NR⁵⁵R⁵⁶, B, Q, and E;
B is selected from the group consisting of —C(═NR⁴⁶)(R⁴⁷), C(O)NR⁴⁰R⁴¹, aryl, heteroaryl, heteroalicyclic, S(O)₂R⁸, C(O)R⁷, XR^{8a}, (C₁₋₆)alkylNR⁴⁰R⁴¹, (C₁₋₆)alkylCOOR^{8b}; wherein said aryl, heteroaryl, and heteroalicyclic are optionally substituted with one to three same or different halogens or from one to three same or different substituents selected from the group F; wherein aryl is napthyl or substituted phenyl; wherein heteroaryl is a mono or bicyclic system which contains from 3 to 7 ring atoms for a mono cyclic system and up to 12 atoms in a fused bicyclic system, including from 1 to 4 heteroatoms; wherein heteroalicyclic is a 3 to 7 membered mono cyclic ring which may contain from 1 to 2 heteroatoms in the ring skeleton and which may be fused to a benzene or pyridine ring;
Q is selected from the group consisting of (C₁₋₆)alkyl and (C₂₋₆)alkenyl; wherein said (C₁₋₆)alkyl and (C₂₋₆)alkenyl are optionally substituted with one to three same or different halogens or from one to three same or different substituents selected from the group consisting of C(O)NR⁵⁵R⁵⁶, hydroxy, cyano and XR⁵⁷;
E is selected from the group consisting of (C₁₋₆)alkyl and (C₂₋₆)alkenyl; wherein said (C₁₋₆)alkyl and (C₂₋₆)alkenyl are independently optionally substituted with a member selected from the group consisting of phenyl, heteroaryl, SMe, SPh,
—C(O)NR₅₆R₅₇, C(O)R₅₇, SO₂(C₁₋₆)alkyl and SO₂Ph; wherein heteroaryl is a monocyclic system which contains from 3 to 7 ring atoms, including from 1 to 4 heteroatoms;
F is selected from the group consisting of (C₁₋₆)alkyl, (C₃₋₇)cycloalkyl, aryl, heteroaryl, heteroalicyclic, hydroxy, (C₁₋₆)alkoxy, aryloxy, (C₁₋₆)thioalkoxy, cyano, halogen, nitro, —C(O)R⁵⁷, benzyl, —NR⁴²C(O)—(C₁₋₆)alkyl, —NR⁴²C(O)—
(C₃₋₆)cycloalkyl, —NR⁴²C(O)-aryl, —NR⁴²C(O)-heteroaryl, —NR⁴²C(O)-heteroalicyclic, a 4, 5, or 6 membered ring cyclic N-lactam, —NR⁴²S(O)₂—(C₁₋₆)alkyl, —NR⁴²S(O)₂—(C₃₋₆)cycloalkyl, —NR⁴²S(O)2-aryl, —NR⁴²S(O)₂-heteroaryl, —NR⁴²S(O)2-heteroalicyclic, S(O)₂(C₁₋₆)alkyl, S(O)₂aryl, —S(O)2NR⁴²R⁴³, NR⁴²R⁴³,
(C₁₋₆)alkylC(O)NR⁴²R⁴³, C(O)NR⁴²R⁴³, NHC(O)NR⁴²R⁴³, OC(O)NR⁴²R⁴³, NHC(O)OR⁵⁴, (C₁₋₆)alkylNR⁴²R⁴³, COOR⁵⁴, and (C₁₋₆)alkylCOOR⁵⁴; wherein said (C₁₋₆)alkyl, (C₃₋₇)cycloalkyl, aryl, heteroaryl, heteroalicyclic, (C₁₋₆)alkoxy, and aryloxy, are optionally substituted with one to nine same or different halogens or from one to five same or different substituents selected from the group G; wherein aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
G is selected from the group consisting of (C₁₋₆)alkyl, (C₃₋₇)cycloalkyl, aryl, heteroaryl, heteroalicyclic, hydroxy, (C₁₋₆)alkoxy, aryloxy, cyano, halogen, nitro,
—C(O)R⁵⁷, benzyl, —NR⁴⁸C(O)—(C₁₋₆)alkyl, —NR⁴⁸C(O)—(C₃₋₆)cycloalkyl, —NR⁴⁸C(O)-aryl, —NR⁴⁸C(O)-heteroaryl, —NR⁴⁸C(O)-heteroalicyclic, a 4, 5, or 6 membered ring cyclic N-lactam, —NR⁴⁸S(O)₂—(C₁₋₆)alkyl, —NR⁴⁸S(O)₂—
(C₃₋₆)cycloalkyl, —NR⁴⁸S(O)₂-aryl, —NR⁴⁸S(O)₂-heteroaryl, —NR⁴⁸S(O)2-heteroalicyclic, sulfinyl, sulfonyl, sulfonamide, NR⁴⁸R⁴⁹, (C₁₋₆)alkyl C(O)NR⁴⁸R⁴⁹, C(O)NR⁴⁸R⁴⁹, NHC(O)NR⁴⁸R⁴⁹, OC(O)NR⁴⁸R⁴⁹, NHC(O)OR^{54′},
(C₁₋₆)alkylNR⁴⁸R⁴⁹, COOR⁵⁴, and (C₁₋₆)alkylCOOR⁵⁴; wherein
aryl is phenyl; heteroaryl is a monocyclic system which contains from 3 to 7 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine;
R⁷ is selected from the group consisting of aryl, heteroaryl, and heteroalicyclic; wherein said aryl, heteroaryl, and heteroalicyclic are optionally substituted with one to three same or different halogens or with from one to three same or different substituents selected from the group F;
wherein for R⁷, R⁸, R^{8a}, R^{8b} aryl is phenyl; heteroaryl is a mono or bicyclic system which contains from 3 to 7 ring atoms for mono cyclic systems and up to 10 atoms in a bicyclic system, including from 1 to 4 heteroatoms; wherein heteroalicyclic is selected from the group consisting of aziridine, azetidine, pyrrolidine, piperazine, piperidine, tetrahydrofuran, tetrahydropyran, azepine, and morpholine;
R⁸ is selected from the group consisting of hydrogen, (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic; wherein said (C₁₋₆)alkyl, (C₃₋₇)cycloalkyl, (C₂₋₆)alkenyl, (C₃₋₇)cycloalkenyl, (C₂₋₆)alkynyl, aryl, heteroaryl, and heteroalicyclic are optionally substituted with one to six same or different halogens or from one to five same or different substituents selected from the group F;
R^{8a} is a member selected from the group consisting of aryl, heteroaryl, and heteroalicyclic; wherein each member is independently optionally substituted with one to six same or different halogens or from one to five same or different substituents selected from the group F;
R^{8b} is selected from the group consisting of hydrogen, (C₁₋₆)alkyl and phenyl;
R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
R⁹, R¹⁰, R¹¹, R¹², R¹³, R¹⁴, R¹⁵, R¹⁶, are each independently selected from the group consisting of hydrogen and (C₁₋₆)alkyl; wherein said (C₁₋₆)alkyl is optionally substituted with one to three same or different halogens;
X is selected from the group consisting of NH or NCH₃, O, and S;
R⁴⁰ and R⁴¹ are independently selected from the group consisting of
(a) hydrogen; (b) (C₁₋₆)alkyl or (C₃₋₇)cycloalkyl substituted with one to three same or different halogens or from one to two same or different substituents selected from the group F; and (c) (C₁₋₆)alkoxy, aryl, heteroaryl or heteroalicyclic; or R⁴⁰ and R⁴¹ taken together with the nitrogen to which they are attached form a member selected from the group consisting of aziridine, azetidine, pyrrolidine, piperazine, 4-NMe piperazine, piperidine, azepine, and morpholine; and wherein said aryl, heteroaryl, and heteroalicyclic are optionally substituted with one to three same or different halogens or from one to two same or different substituents selected from the group F; wherein for R⁴⁰ and R⁴¹ aryl is phenyl; heteroaryl is a monocyclic system which contains from 3 to 6 ring atoms, including from 1 to 4 heteroatoms; heteroalicyclic is selected from the group consisting of aziridine, azetidine, pyrrolidine, piperazine, piperidine, tetrahydrofuran, tetrahydropyran, azepine, and morpholine; provided when B is C(O)NR⁴⁰R⁴¹, at least one of R⁴⁰ and R⁴¹ is not selected from groups (a) or (b);
R⁴² and R⁴³ are independently selected from the group consisting of hydrogen, (C₁₋₆)alkyl, allyl, (C₁₋₆)alkoxy, (C₃₋₇)cycloalkyl, aryl, heteroaryl and heteroalicyclic; or R⁴² and R⁴³ taken together with the nitrogen to which they are attached form a member selected from the group consisting of aziridine, azetidine, pyrrolidine, piperazine, 4-NMe piperazine, piperidine, azepine, and morpholine; and wherein said (C₁₋₆)alkyl, (C₁₋₆)alkoxy, (C₃₋₇)cycloalkyl, aryl, heteroaryl, and heteroalicyclic are optionally substituted with one to three same or different halogens or from one to two same or different substituents selected from the group G; wherein for R⁴² and R⁴³ aryl is phenyl; heteroaryl is a monocyclic system which contains from 3 to 6 ring atoms, including from 1 to 4 heteroatoms; heteroalicyclic is a member selected from the group consisting of aziridine, azetidine, pyrrolidine, piperazine, piperidine, tetrahydrofuran, tetrahydropyran, azepine, and morpholine;
R⁴⁶ is selected from the group consisting of H, OR⁵⁷, and NR⁵⁵R⁵⁶;
R⁴⁷ is selected from the group consisting of H, amino, halogen, phenyl, and (C₁₋₆)alkyl;
R⁴⁸ and R⁴⁹ are independently selected from the group consisting of hydrogen, (C₁₋₆)alkyl and phenyl;
R⁵⁰ is selected from the group consisting of H, (C₁₋₆)alkyl, (C₃₋₆)cycloalkyl, and benzyl; wherein each of said (C₁₋₆)alkyl, (C₃₋₇)cycloalkyl and benzyl are optionally substituted with one to three same or different halogen, amino, OH, CN or NO₂;
R⁵⁴ is selected from the group consisting of hydrogen and (C₁₋₆)alkyl;
R^{54′} is (C₁₋₆)alkyl;
R⁵⁵ and R⁵⁶ are independently selected from the group consisting of hydrogen and (C₁₋₆)alkyl; and
R⁵⁷ is selected from the group consisting of hydrogen, (C₁₋₆)alkyl and phenyl; and
J is selected from the group consisting of: 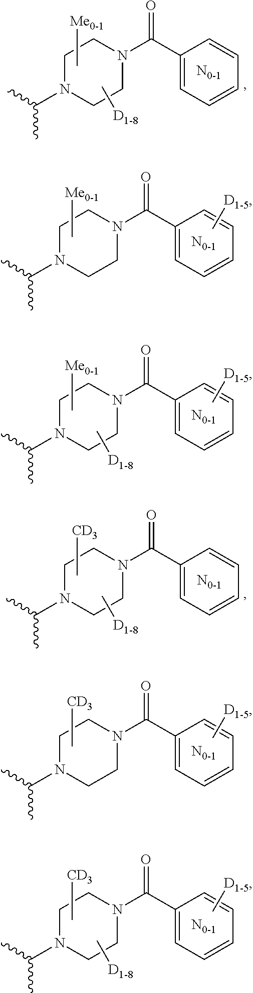 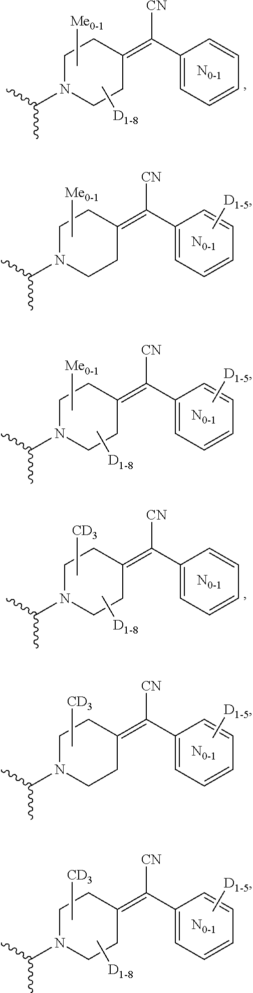 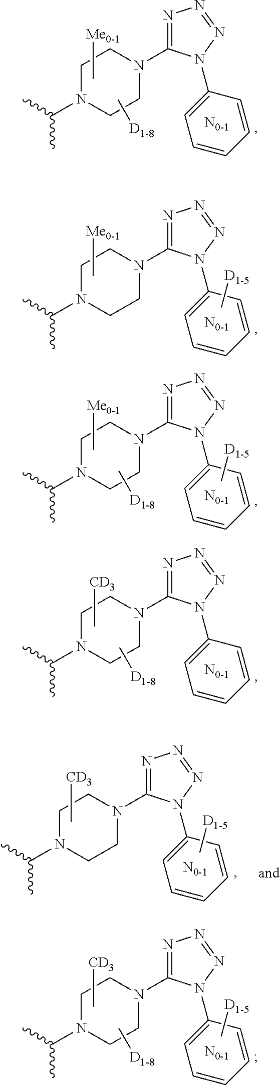
wherein Me represents methyl, and D represents deuterium.

2. A compound which is selected from the group consisting of 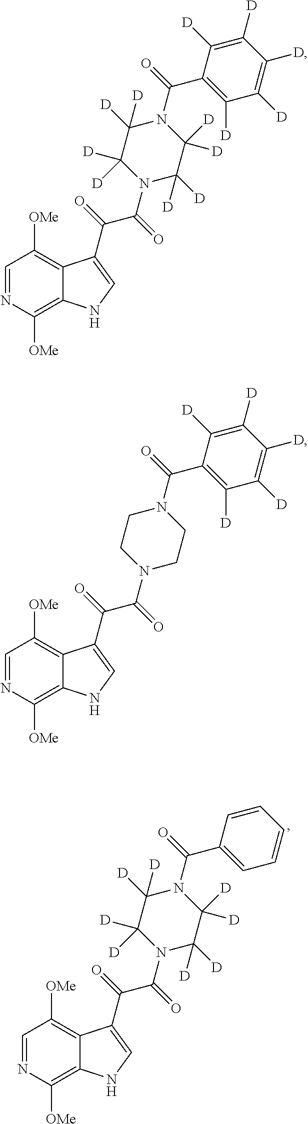 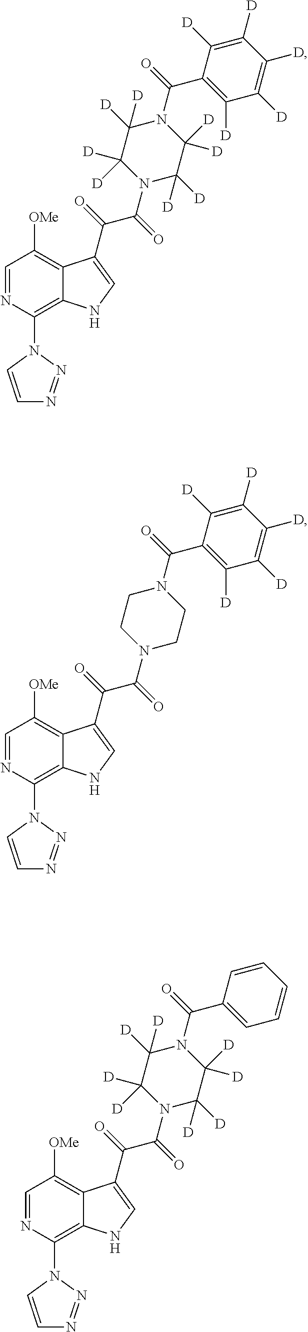 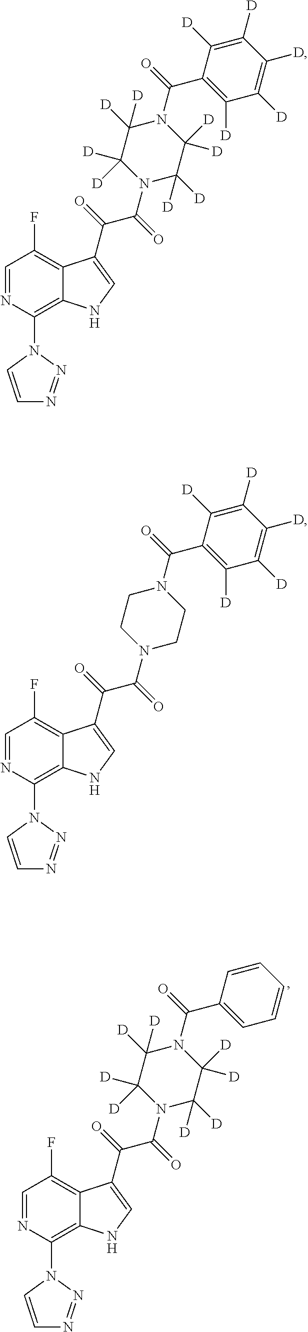 [chemical structure]
